(19) United States
(12) Patent Application Publication
(10) Pub. No.: US 2026/0071278 A1
(43) Pub. Date: Mar. 12, 2026

(54) SALIVA-BASED DETECTION OF ORAL CANCER

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Diana Rose E. RANOA, Champaign, IL (US)

(73) Assignee: THE BOARD OF TRUSTEES OF THE UNIVERSITY OF ILLINOIS, Urbana, IL (US)

(21) Appl. No.: 18/871,555

(22) PCT Filed: Jun. 12, 2023
(86) PCT No.: PCT/US2023/025041

§ 371 Date: Dec. 4, 2024

Related U.S. Application Data
(60) Provisional application No. 63/351,031, filed on Jun. 10, 2022.

(51) Int. Cl. C12Q 1/6886; C12Q 1/70

(52) U.S. Cl. CPC C12Q 1/6886; C12Q 1/708; C12Q 2600/156; C12Q 2600/158

(57) ABSTRACT

The disclosure provides for methods of detecting target polynucleotides in a biological sample from a subject including the steps of extracting the target polynucleotides from the biological sample and subjecting the biological sample to conditions that amplify the target polynucleotides in the biological sample using a polymerase chain reaction (PCR) and detecting the target polynucleotides in biological sample. The methods may be used to detect Human Papilloma Virus (HPV) mediated squamous cell carcinoma or non-HPV mediated squamous cell carcinoma.

### RELATED APPLICATIONS

This application claims priority under 35 U.S.C. § 119(e) to U.S. Provisional Patent Application No. 63/351,031, filed Jun. 10, 2022, which is incorporated herein by reference.

### BACKGROUND OF THE INVENTION

Head-and-neck squamous cell carcinomas (HNSCC) afflict 500,000 new patients each year worldwide, and in the U.S., there are 40,000 new cases annually, with approximately 8,000 deaths. Although HNSCC affects many individuals, effective treatment options for patients with advanced stage disease are exceedingly limited, making intervention via early detection and local surgical resection the preferred treatment modality.

Arising from the oral cavity, oropharynx, hypopharynx, larynx, or nasopharynx, 40% of HNSCC patients present with regional nodal involvement (stage IVA or B), and 10% present with distant metastases (stage IVC). The prognosis for patients with metastatic disease, bone invasion, or recurrent disease is quite poor (median survival of 6 to 9 months) even with multimodal treatment of surgery, radiation, and chemotherapy. Immunotherapy for HNSCC shows some promise but will likely be limited by low response rates, and while targeted therapies—drugs that exploit specific alterations in cancer cells—have revolutionized the treatment of some cancers (e.g., Chronic myelogenous leukemia) and have led to survival increases for certain subtypes of other cancers (e.g., Melanoma, ovarian, breast), the activity of targeted therapies for HNSCC has not been clinically impactful; EGFR blockade with cetuximab exerts minimal activity (response rate ˜15% and progression free interval <3 months) in patients diagnosed with metastatic/recurrent disease. As such, effective treatment options for these patients with advanced stage disease are exceedingly limited, making intervention via early detection and local surgical resection the only curative modality. Unfortunately, screening for oral cancer is still rudimentary (via visual inspection) and not routine practice.

Current protocols for screening for head and neck cancer is through a visual and physical examination of the nose, mouth, and throat by a medical care provider. If there are signs pointing to head or neck cancer, more tests will be done (e.g., panendoscopy, biopsy, CT/MRI/PET scan, Barium swallow, and chest x-rays). And even after these tests, HNSCC still needs to be differentiated between HPV-positive and -negative HNSCC using methods such as P16 immunohistochemistry, fluorescence in situ hybridization, and genetic analyses of the HPV gene from histopathological and liquid biopsy specimens.

Other methods have used saliva to screen for HNSCC, but the methods used (digital PCR and safe-sequencing system) are not convenient and not compatible with standard laboratory technologies and protocols. At present, there are no FDA-approved tests to detect HPV DNA or mRNA in saliva; however, salivary rinse or swab tests for HPV-positive HNSCC have been used in research settings.

Accordingly, there is a need for a simple, convenient method using saliva or other bodily fluid for detecting HPV-positive and -negative SCC and HNSCC that could be performed yearly (or more frequently) to detect HNSCC before it locally advanced. The present disclosure satisfies these needs.

### SUMMARY OF THE INVENTION

This disclosure provides for methods of detecting target polynucleotides in a biological sample from a subject comprising the steps of extracting polynucleotides from a biological sample, amplifying the extracted polynucleotides from the biological sample using a polymerase chain reaction (PCR) method, and detecting target polynucleotides in the amplified polynucleotides from the biological sample. In some aspects, the presence or absence of detected target polynucleotides in the sample may indicate the presence or absence of a certain type of cancer in the subject.

In one embodiment, a method of detecting Human Papillomavirus (HPV) mediated Squamous Cell Carcinoma (SCC) in a subject comprising: extracting polynucleotides from a biological sample from a subject; amplifying the extracted polynucleotides from the biological sample using a polymerase chain reaction (PCR) method; detecting target polynucleotides in the amplified polynucleotides from the biological sample, wherein the target polynucleotides comprise one or more of an E6 gene of HPV16, an E7 gene of HPV16, and an E7 gene of HPV18; and wherein detecting the target polynucleotides indicates the presence of SCC in the subject.

In one embodiment, a method of detecting Human papilloma Virus (HPV) mediated Head and Neck Squamous Cell Carcinoma (SCC) comprising: extracting deoxyribonucleic acid or ribonucleic acid from a saliva sample from a subject; subjecting the extracted deoxyribonucleic acid or ribonucleic acid to conditions that amplify the extracted ribonucleic acid using quantitative reverse transcription polymerase chain reaction (RT-qPCR); and detecting target polynucleotides in the amplified deoxyribonucleic acid or ribonucleic acid, wherein the target polynucleotides comprise an E6 gene of HPV16, an E7 gene of HPV16, and an E7 gene of HPV18; wherein detection of the target polynucleotides indicates a presence or absence of HPV mediated SCC in the biological sample.

In one embodiment, a method of detecting non-Human Papilloma Virus mediated Squamous Cell Carcinoma (SCC) comprising: extracting polynucleotides from a biological sample from a subject; amplifying the polynucleotides in the biological sample using a polymerase chain reaction (PCR) method; and detecting target polynucleotides in the amplified polynucleotides, wherein the target polynucleotides comprise one or more polynucleotide sequences of TP53, CDKN2A, CCND1, EGFR, PIK3CA, HRAS, KRAS, NRAS, and FBXW7; wherein a change in expression level of the target polynucleotides or a change in copy number of the target polynucleotides as compared to a biological sample from a reference subject known to be SCC free indicates a presence or absence of non-HPV mediated SCC in the biological sample.

In some embodiments, the change in expression level of the target polynucleotides is determined according to the steps of: measuring a change in Ct (ΔCt) value for each of the target polynucleotides and a second polynucleotide, wherein the second polynucleotide comprises one or more of RNAseP, 18S, and beta-actin for both the subject and the reference subject known to be SCC free; calculating the difference in ΔCt value (ΔΔCt) of the subject and the reference subject known to be SCC free obtained from the measuring step; computing a 2^{-ΔΔCt} to produce a final value for each of the one or more target polynucleotides and the second polynucleotide; comparing the final value for each of the one or more target polynucleotides to the second polynucleotide to form a comparison ratio, wherein the comparison ratio indicates a fold change in expression of each of the one or more target polynucleotides, and wherein a comparison ratio of 1 or greater indicates the presence of SCC and a comparison ratio of less than 1 indicates the absence of SCC in the biological sample.

In some embodiments, a method of detecting a cancer in a subject may comprise measuring a fold change in gene copy number of the target polynucleotides, wherein an increase in the gene copy number of the target polynucleotides compared to a wild-type gene copy number of the target polynucleotides indicates the presence of a cancer such as SCC.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Detection of genes important for HPV-mediated head-and-neck cancer from saliva. A-B) Plasmid DNA encoding either HPV16 or HPV18 was spiked into saliva, heated at 95° C., cooled, mixed 1:1 with 2×TBE buffer, and used as a template for PCR using primers targeting HPV16_E7 (A) and HPV18_E7 (B). C) Saliva samples were obtained from three HNSCC patients from Carle Hospital, one sample from Patient 1, and two different samples from Patients 2 & 3. RNA was isolated and RT-qPCR was performed, showing that these patients were positive for HPV16 (but not HPV18). D) Genomic DNA was isolated from the three HNSCC patient saliva samples from Carle Hospital and used as template for qPCR, results also demonstrating that patients were positive for HPV16 (but not HPV18).

FIG. 2. Detection of HPV16 using 2 different primers specific for the E7 and E6 oncogenes. Samples were run in triplicates and scored as positive for target gene if all 3 replicates were detected.

FIG. 3. Detection of CDKN2A and housekeeping genes (18S rRNA and β-actin) following RT-qPCR of total RNA isolated from three HNSCC patient saliva samples from Carle Hospital. A) Raw Ct values obtained using two primer pairs targeting CDKN2A (“CDKN2A_123_NA” and “CDKN2A_129_NA”) and the housekeeping genes 18srRNA and β-actin. B) Fold change of CDKN2A expression was calculated relative to the housekeeping genes and the normal HPV-negative saliva sample using the 2^{(ΔΔCt)} method. Black dashed line is drawn at fold change=1.

FIG. 4. Examples of standard curve generated from known amounts of either wild type gene target (e.g., PIK3CA) in pDONR223 plasmid construct purchased from Addgene or synthetic fragments of RNase P purchased from IDT. The equation of the line was calculated using a non-linear fit of a semi-log line (X is log, Y is linear) on GraphPad Prism v9.2.0.

FIG. 5. Fold change of TP53 (A), PIK3CA (B), HRAS (C), NRAS (D), and FBXW7 (E) expression in patient saliva samples was calculated relative to the 18S rRNA housekeeping gene and the HPV-negative/no cancer saliva sample using the 2^{-(ΔΔCt)} method. Black dashed line is drawn at fold change=1. Patient samples with fold change greater than 2 were scored as positive for the respective gene target. All samples were run in triplicates.* Not detected.

FIG. 6. Gene copy number of CDKN2A (A), TP53 (B), PIK3CA (C), HRAS (D), NRAS (E), and FBXW7 (F) in patient saliva samples was extrapolated from their respective standard curves using the equation reported in Table III. For CDKN2A (A), the hatched bars represent patients that tested positive for CDKN2A in IHC staining of biopsy samples. All samples were run in triplicates. * Not detected.

FIG. 7. Workflow for detection of head-and-neck cancer from saliva, enabling detection of HPV-mediated and non-HPV-mediated forms of this cancer.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991).

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment). For example, and effective amount of buffering agent may comprise combining a biological sample and the buffering agent in a ratio of about 1:3 w/w to about 3:1 w/w, and an effective amount of non-ionic detergent may comprise a final concentration of about 0.25% to about 1% w/w, or about 0.5% w/w.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

The terms “polynucleotide” and “nucleic acid” are used interchangeably and mean at least two or more ribo- or deoxy-ribo nucleic acid base pairs (nucleotide) linked which are through a phosphoester bond or equivalent. The nucleic acid includes polynucleotide and polynucleoside. The nucleic acid includes a single molecule, a double molecule, a triple molecule, a circular molecule, or a linear molecule. Examples of the nucleic acid include RNA, DNA, cDNA, a genomic nucleic acid, a naturally existing nucleic acid, and a non-natural nucleic acid such as a synthetic nucleic acid but are not limited. Short nucleic acids and polynucleotides (e.g., 10 to 20, 20 to 30, 30 to 50, 50 to 100 nucleotides) are commonly called “oligonucleotides” or “probes” of single-stranded or double-stranded DNA.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, J M B, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “primer” as used herein refers to a short polynucleotide that hybridizes to a target polynucleotide sequence and serves as the starting point for synthesis of new polynucleotides.

The term “amplification” refers to an increase in the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The product of amplification can be characterized by such techniques as electrophoresis, restriction endonuclease cleavage patterns, oligonucleotide hybridization or ligation, and/or nucleic acid sequencing. In some embodiments, the methods provided herein can include a step of producing an amplified nucleic acid under isothermal or thermal variable conditions.

The term “multiplex” refers to the use of more than one pair of primers intended to amplify multiple target gene segments simultaneously within a single tube. In this manner, all the primers may be contained within one tube to which a sample is introduced or positioned. All desired influenza virus and control gene segments are then amplified via the plurality of forward and reverse primers within the tube.

The term “complement” as used herein means the complementary sequence to a nucleic acid according to standard Watson/Crick base pairing rules. A complement sequence can also be a sequence of RNA complementary to the DNA sequence or its complement sequence and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

### Embodiments of the Invention

The disclosure provides a non-invasive method of detecting target polynucleotides obtained from bodily fluid samples of a subject having or suspected of having a Squamous Cell Carcinoma (SCC), and in particular, a head and neck SCC (HNSCC). In some embodiments, the SCC or HNSCC may be mediated by various strains of Human Papilloma Virus (HPV) or through various insertions or deletion of the subject genome. In other embodiments, the disclosure provides for non-invasive methods of detecting target polynucleotides obtained from bodily fluid samples of a subject having or suspected of having non-HPV mediated SCC.

In various embodiments of the present invention, oligonucleotide primers and/or probes are used in the methods described herein to amplify and detect target polynucleotide sequences, the detection of which (in some instances, detection over a certain threshold value) are indicative of the presence of SCC or HNSCC. In other embodiments, detection of target polynucleotides is indicative of the presence of, for example, one or more of SCC, HNSCC, adenoid/pseudo glandular squamous cell carcinoma, intraepidermal squamous cell carcinoma, large cell keratinizing squamous cell carcinoma, large cell non-keratinizing squamous cell carcinoma, lymphoepithelial carcinoma, papillary squamous cell carcinoma, papillary thyroid carcinoma, small cell keratinizing squamous cell carcinoma, spindle cell squamous cell carcinoma, and verrucous squamous-cell carcinoma.

In certain embodiments, a method of detecting target polynucleotides in a biological sample may include the steps of extracting the polynucleotides (e.g., RNA or DNA) from the biological sample, amplifying the extracted polynucleotides in the biological sample using a polymerase chain reaction (PCR) method, and detecting target polynucleotides in the amplified polynucleotides from the biological sample. In some embodiments, only the target polynucleotides are amplified from the extracted polynucleotides.

Preferably, the biological sample may comprise a bodily fluid such as urine, saliva, ascites fluid, vaginal fluid, cervical swabs, blood, serum, plasma, or a combination thereof. In other embodiments, the biological sample is obtained from a mucosal membrane of the subject. In preferred embodiments, the bodily fluid is saliva. In other embodiments, the biological sample comprises a tissue sample or liquid biopsy.

In some embodiments, polynucleotides may be extracted from the biological sample for use in a PCR reaction or other analysis. Polynucleotide extraction from biological samples is well known in the art. For example, RNA may be isolated by a variety of methods and reagents including (but not limited to) guanidinium thiocyanate-phenol-chloroform extraction (e.g., with TRIzol® reagent, also known as TRI Reagent), hypotonic lysis, and carboxyl (COOH) bead capture. The principle of RNA isolation is based on cell/tissue lysis, followed by extraction, precipitation, and washing. Alternatively, RNA may be isolated using commercially available kits, such as, but not limited to, TRIzol® or QIAGEN resin technology (for example, QIAGEN RNeasy Plus Mini Kit) can also be used to isolate the RNA polynucleotides. Similarly, DNA extraction from biological samples is also well known in the art and are described, for example, in Molecular Cloning: A Laboratory Manual, J. Sambrook et al., 4th edition, Cold Spring Harbor Laboratory Press, 2012.

After the biological sample is collected and processed according to the methods disclosed herein, the target polynucleotides may be amplified by various methods known in the art. Preferably, PCR or a derivative method thereof, is used to amplify nucleic acids of interest (Ghannam, M. G. et al. (2020) “Biochemistry, Polymerase Chain Reaction (PCR),” StatPearls Publishing, Treasure Is; pp 0.1-4; Lorenz, T. C. (2012) “Polymerase Chain Reaction: Basic Protocol Plus Troubleshooting and Optimization Strategies,” J. Vis. Exp. 2012 May 22; (63):e3998; pp. 1-15).

Briefly, in PCR, two primer sequences are prepared that are complementary to regions on opposite complementary strands of the marker sequence. An excess of deoxynucleotide triphosphates is added to a reaction mixture along with a DNA polymerase, e.g., Taq polymerase.

If the target sequence is present in a sample, the primers will bind to the sequence and the polymerase will cause the primers to be extended along the target sequence by adding on nucleotides. By raising and lowering the temperature of the reaction mixture, the extended primers will dissociate from the target nucleic acid to form reaction products, excess primers will bind to the target nucleic acid and to the reaction products and the process is repeated, thereby generating amplification products. Cycling parameters can be varied, depending on the length of the amplification products to be extended. An internal positive amplification control (IAC) can be included in the sample, utilizing oligonucleotide primers and/or probes. The IAC can be used to monitor both the conversion process and any subsequent amplification.

A person of ordinary skill in the art may design and prepare primers that are appropriate for amplifying a target sequence in view of the information disclosed herein. The length of the amplification primers for use in the disclosed methods is dependent upon on several factors. These include the nucleotide sequence identity and the temperature at which these nucleic acids are hybridized or used during nucleic acid amplification. The considerations necessary to determine a preferred length for an amplification primer of a sequence identity are well known to the person of ordinary skill in the art.

For example, primers that amplify a nucleic acid molecule can be designed using, for example, a computer program such as OLIGO® (Molecular Biology Insights, Inc., Cascade, Colo.). Important features when designing oligonucleotides to be used as amplification primers include, but are not limited to, an appropriate size amplification product to facilitate detection (e.g., by electrophoresis or real-time PCR), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 40 nucleotides in length.

In preferred embodiments, the PCR technique used to amplify a target polynucleotide is real-time quantitative PCR (RT-qPCR) or quantitative PCR (qPCR). Quantitative PCR is characterized in that a PCR product is marked and tracked through a fluorescent dye or a specific probe marked by fluorescence to carry out a real-time monitoring reaction, and the product is analyzed using software adapted to monitor the reaction, such that the initial concentration of a target polynucleotide in a sample may be calculated. A reverse transcription reaction is involved in the PCR reaction process when the target polynucleotide is an RNA nucleic acid and the resultant amplified product may be analyzed using CT-values (see, for example, Chan et al., J Clin Microbiol. 2020 May; 58(5): e00310-20).

In some embodiments, the polynucleotide may include a target polynucleotide that may comprise one or more of a human papilloma virus (HPV) such as, HPV6, HPV11, HPV16, HPV18, HPV33, HPV35, HPV39, HPV51, HPV52, HPV56, HPV58, HPV59, HPV68, and HPV69. In certain preferred embodiments, the HPV is HPV 16 and/or HPV 18. In some embodiments, the HPV genes comprise, consist essentially of, or consist of one or more of E6 of HPV16, E7 of HPV16, E7 of HPV18, or RNA products thereof. In some embodiments, the HPV genes comprise E6 of HPV16, E7 of HPV16, E7 of the HPV18, or RNA products thereof.

In some embodiments, the HPV target genes comprise an RNA product of the gene. Exemplary primers that may be used with the embodiments of the invention are listed in Table II-V.

In some embodiments, detection of any one of the HPV genes indicates the presence of Squamous Cell Carcinoma (SCC) in the biological sample. In particular, detection of any one of the HPV genes indicates the presence of head and neck Squamous Cell Carcinoma (HNSCC) in the biological sample. In other embodiments, amplification of target polynucleotides from, for example, saliva, vaginal swabs, pap smear, ascites, etc. may be used to detect cervical cancer, anal cancer, penile cancer, vaginal, and vulvar cancer. Other SCCs include, but are not limited to, adenoid/pseudo glandular squamous cell carcinoma, intraepidermal squamous cell carcinoma, large cell keratinizing squamous cell carcinoma, large cell non-keratinizing squamous cell carcinoma, lymphoepithelial carcinoma, papillary squamous cell carcinoma, papillary thyroid carcinoma, small cell keratinizing squamous cell carcinoma, spindle cell squamous cell carcinoma, and verrucous squamous-cell carcinoma.

In some embodiments, a method of detecting Human Papillomavirus (HPV) mediated Squamous Cell Carcinoma (SCC) in a subject comprising: extracting polynucleotides from a biological sample from a subject; amplifying the extracted polynucleotides from the biological sample using a polymerase chain reaction (PCR) method; detecting target polynucleotides in the amplified polynucleotides from the biological sample, wherein the target polynucleotides comprise one or more of E6 gene of HPV16, E7 of HPV16, and E7 of HPV18; and wherein detecting the target polynucleotides indicates the presence of SCC in the subject. In some embodiments, the SCC is Head and Neck SCC or a cervical cancer.

In some embodiments, a method of detecting Human papilloma Virus (HPV) mediated Head and Neck Squamous Cell Carcinoma (SCC) or cervical cancer comprising: extracting deoxyribonucleic acid or ribonucleic acid from a saliva sample from a subject; subjecting the extracted deoxyribonucleic acid or ribonucleic acid to conditions that amplify the extracted ribonucleic acid using quantitative reverse transcription polymerase chain reaction (RT-qPCR); and detecting target polynucleotides in the amplified deoxyribonucleic acid or ribonucleic acid, wherein the target polynucleotides comprise one or more of an E6 gene of HPV16, an E7 gene of HPV16, and an E7 gene of HPV18; wherein detection of the target polynucleotides indicates a presence or absence of HPV mediated Head and Neck SCC or cervical cancer in the biological sample.

In some embodiments, a method of detecting Human papilloma Virus (HPV) mediated Head and Neck Squamous Cell Carcinoma (HNSCC) or cervical cancer comprising: extracting polynucleotides from a saliva sample from a subject; amplifying the extracted polynucleotides in the saliva sample using reverse transcription quantitative real-time PCR; and detecting target polynucleotides in the amplified polynucleotides, wherein the target polynucleotides consist of an E6 gene of HPV16, an E7 gene of HPV16, and an E7 gene of HPV18; wherein detection of the target polynucleotides indicates a presence or absence of the HPV mediated HNSCC or the cervical cancer in the biological sample.

In some embodiments, the target polynucleotide may be a portion of a gene. Exemplary portions of certain target polynucleotides are listed, for example, in Table V. By way of example, in some embodiments, the E6 gene of the HPV16 genome (NCBI Accession No. U89348.1) comprises nucleotides 83-559 or SEQ ID NO: 1. In some embodiments, a target polynucleotide that is the target of amplification and detection for the E6 gene of HPV16 comprises nucleotides 101-219 of SEQ ID NO: 1. In some embodiments, a target polynucleotide that is the target of amplification and detection for the E7 gene of HPV16 comprises nucleotides 667-774 of SEQ ID NO: 1.

In some embodiments, the E6 gene of the HPV16 genome (NCBI Accession No. LC718903.1) comprises nucleotides 83-560 of SEQ ID NO: 3. In some embodiments, a target polynucleotide that is the target of amplification and detection for the E6 gene of HPV16 comprises nucleotides 101-219 of SEQ ID NO: 3. In some embodiments, the E7 gene HPV16 genome (NCBI Accession No. LC718903.1) corresponds to nucleotides 562-858 of SEQ ID NO: 3. In some embodiments, a target polynucleotide that is the target of amplification and detection for the E7 gene of HPV16 comprises nucleotides 667-774 of SEQ ID NO: 3.

In some embodiments, the E7 gene of the HPV18 genome (NCBI Accession No. LC509006.1) comprises nucleotides 590-907 of SEQ ID NO: 2. In some embodiments, a target polynucleotide that is the target of amplification and detection for the E7 gene of HPV18 comprises nucleotides 592-665 of SEQ ID NO: 2.

In some embodiments, the E6 gene of HPV16 comprises SEQ ID NO: 4, and a target polynucleotide that is the target of amplification and detection for the E6 gene comprises nucleotides 19-137 of SEQ ID NO: 4. In some embodiments, the E7 gene of HPV16 comprises SEQ ID NO: 5, and a target polynucleotide that is the target of amplification and detection for the E7 gene comprises nucleotides 106-212 of SEQ ID NO: 5. In some embodiments, the E7 gene of HPV18 comprises SEQ ID NO: 6, and a target polynucleotide that is the target of amplification and detection for the E7 gene comprises nucleotides 3-76 of SEQ ID NO: 6.

In some embodiments, the target polynucleotides comprise, consist essentially of, or consist of one or more HPV genes and/or one or more human genes such as one or more of human TP53, CDKN2A, CCND1, EGFR, PIK3CA, HRAS, KRAS, NRAS, and FBXW7. In some embodiments, the target polynucleotide may be a portion of a gene. Exemplary portions of certain target polynucleotides are listed, for example, in Table V. For example, in some embodiments, a portion of the p16/CDKN2A gene that is the target of amplification and detection comprises nucleotides 1-129 of SEQ ID NO: 7 (which corresponds to nucleotides 188262-188391 of NCBI accession No. AB060808.1). In some embodiments, a portion of the TP53 gene that is the target of amplification and detection comprises nucleotides 2490-2612 and/or 2510-2645 of SEQ ID NO: 8. In some embodiments, a portion of the PIK3CA gene that is the target of amplification and detection comprises nucleotides 491-609 and/or 3369-3497 of SEQ ID NO: 9. In some embodiments, a portion of the HRAS gene that is the target of amplification and detection comprises nucleotides 38-160 of SEQ ID NO: 10 and/or 1204-3261 of SEQ ID NO: 11. In some embodiments, a portion of the NRAS gene that is the target of amplification and detection comprises nucleotides 4864-4988 and/or 2633-2754 of SEQ ID NO: 12. In some embodiments, a portion of the KRAS gene that is the target of amplification and detection comprises nucleotides 1-567 of SEQ ID NO: 13. In some embodiments, a portion of the FBXW7 gene that is the target of amplification and detection comprises nucleotides 1625-1753 of SEQ ID NO: 14. In some embodiments, a portion of the CCND1 gene that is the target of amplification and detection comprises nucleotides 280-856 and/or 296-370 of SEQ ID NO: 15. In some embodiments, a portion of the EFGR gene that is the target of amplification and detection comprises nucleotides 638-846, and/or 3491-3693, and/or 785-846 of SEQ ID NO: 16.

In other embodiments, the one or more human genes may be a subset or a single gene of those described above. For example, in some embodiments, the one or more human genes are TP53, CDKN2A, CCND1, EGFR, PIK3CA, HRAS, KRAS, NRAS, and FBXW7; or TP53, CDKN2A, CCND1, EGFR, PIK3CA, HRAS, KRAS, and NRAS; or TP53, CDKN2A, CCND1, EGFR, PIK3CA, HRAS, and KRAS; or TP53, CDKN2A, CCND1, EGFR, PIK3CA, and HRAS; or TP53, CDKN2A, CCND1, EGFR, and PIK3CA; or TP53, CDKN2A, CCND1, and EGFR; or TP53, CDKN2A, and CCND1; or TP53 and CDKN2A. Exemplary NCBI accession numbers for the references sequences are listed in Table VI.

In other embodiments, the one or more human genes are TP53, CDKN2A, PIK3CA, HRAS, NRAS, and FBXW7; or TP53, CDKN2A, PIK3CA, HRAS, and NRAS; or TP53, CDKN2A, PIK3CA, and HRAS; or TP53, CDKN2A, and PIK3CA; or TP53 and CDKN2A.

In some embodiments, a second polynucleotide that is detected may include one or more of RNAseP, ribosomal protein 18S, beta-actin, GAPDH, or other genes termed “housekeeping” genes such as those described in Panina et al., Scientific Reports volume 8, Article number: 8716 (2018). In one embodiment, the target polynucleotide is a polynucleotide encoding CDKN2A and the second polynucleotide is a polynucleotide encoding ribosomal protein 18S, beta-actin, or other housekeeping gene. Sequences of the housekeeping genes or the like are known in the art and may be found, for example, in the NCBI database. Any portion of the reference genes may be amplified for comparison to the target genes.

In some embodiments, the target polynucleotides comprise an RNA product of one or more human genes such as one or more of human TP53, CDKN2A, CCND1, EGFR, PIK3CA, HRAS, KRAS, NRAS, and FBXW7. In some embodiments, a second polynucleotide that is detected may include an RNA product of one or more of RNAseP, ribosomal protein 18S, beta-actin, or other genes housekeeping genes.

In one embodiments, a method of detecting Squamous Cell Carcinoma (SCC) in a subject comprises the steps of: extracting polynucleotides from a biological sample from a subject; amplifying the extracted polynucleotides from the biological sample using a polymerase chain reaction (PCR) method; detecting target polynucleotides in the amplified polynucleotides from the biological sample, wherein the target polynucleotides comprise one or more of E6 gene of HPV16, E7 of HPV16, and E7 of HPV18; and wherein detecting the target polynucleotides indicated the presence of SCC in the subject; or wherein the target polynucleotides comprise one or more of TP53, CDKN2A, CCND1, EGFR, PIK3CA, HRAS, KRAS, NRAS, and FBXW7; and a wherein a change in expression level of the target polynucleotides or a change in copy number of the target polynucleotides as compared to a biological sample from a reference subject known to be SCC free indicates a presence or absence of SCC in the biological sample.

Amplification of nucleic acids can be detected by several methods well-known in the art such as gel electrophoresis, column chromatography, hybridization with a probe, sequencing, melting curve analysis, or “real-time” detection.

In one approach, sequences from one or more target polynucleotides or fragments thereof are amplified in the same reaction vessel (i.e., “multiplex PCR”). Detection can take place by measuring the endpoint of the reaction or in “real time.” For real-time detection, primers and/or probes may be detectably labeled to allow differences in fluorescence when the primers become incorporated or when the probes are hybridized, for example, and amplified in an instrument capable of monitoring the change in fluorescence during the reaction. Real-time detection methods for nucleic acid amplification are well known and include, for example, the TaqMan® system and the use of intercalating dyes (i.e., SYBR Green) for double stranded nucleic acid.

In some embodiments, sequences from one or more target polynucleotides or fragments thereof are each amplified in separate reaction vessels.

In some embodiments, amplified nucleic acids are detected by hybridization with a specific probe. Probe oligonucleotides, complementary to a portion of the amplified target sequence may be used to detect amplified fragments. Hybridization may be detected in real time or in non-real time. Designing oligonucleotides to be used as hybridization probes can be performed in a manner similar to the design of primers. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 60 nucleotides in length. In some embodiments, hybridization probes may be used to identify a target polynucleotide.

Exemplary probes that may be detectably labeled by methods known in the art include, e.g., fluorescent dyes (e.g., Cy5®, Cy3®, FITC, rhodamine, lanthamide phosphors, Texas red, FAM, JOE, SYBR Green Master Mix, Cal Fluor Red 610®, Quasar 670®), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., DYNABEADS), biotin, digoxigenin, or haptens and proteins for which antisera or monoclonal antibodies are available.

Some embodiments include the use of a multiplex RT-qPCR assay. For example, certain embodiments of a method of the disclosure may comprise extracting polynucleotides (e.g., RNA) from a biological sample (e.g., saliva) and subjecting the resultant test sample to conditions that amplify a plurality of target polynucleotides in the test sample using RT-qPCR. The method further may comprise analyzing the test sample for the presence of amplified target polynucleotides in the test sample.

In some embodiments, the RT-qPCR assay may involve either two-step or one-step RT-qPCR reaction. In a two-step process, purified total RNA are first reverse transcribed to complementary DNA (cDNA) using Superscript VILO Master Mix (ThermoFisher) followed by the qPCR reaction using Power SYBR Green (ThermoFisher). For example, 2 uL (up to 16 uL) total RNA+4 uL Superscript VILO master mix will be mixed in 20 uL total reaction volume designated wells. The assay may be performed in 384-well reaction plates in a QuantStudio 7 system (Applied Biosciences). The reverse transcription may be conducted using the standard mode, consisting of, for example, a hold stage at 60° C. for 10 min, a hold stage at 50° C. for 10 min, and a hold stage at 85° C. for 5 min. The 20 uL reaction volume will be diluted to 170 uL total volume by the addition of 150 uL deionized water. From this diluted cDNA, 2.0 uL will be mixed with 5 uL 2×SYBR Green and 0.5 uL 5 uM primer pairs (forward+reverse) and filled to 10 uL by addition of 2.5 uL deionized water. The assay may be performed in 384-well reaction plates in a QuantStudio 7 system (Applied Biosciences). The qPCR may be conducted using the standard mode, consisting of, for example, a hold stage at 95° C. for 10 min, followed by 40 cycles of a PCR stage at 95° C. for 15 sec then 60° C. for 60 sec; with a 1.6° C./sec ramp up and ramp down rate. Alternatively, in a one-step process, up to 4.42 uL purified total RNA may be mixed with 5 uL Power SYBR Green RT-PCR Mix, 0.08 uL RT enzyme mix, and 0.5 uL 5 uM primer pairs (forward+reverse) and filled to 10 uL by addition of deionized water. The assay may be performed in 384-well reaction plates in a QuantStudio 7 system (Applied Biosciences). The RT-qPCR may be conducted using the standard mode, consisting of, for example, a hold stage at 48° C. for 30 min, a hold stage at 95° C. for 10 min, followed by 40 cycles of a PCR stage at 95° C. for 15 sec then 60° C. for 60 sec; with a 1.6° C./sec ramp up and ramp down rate. At the end of the PCR run, an optional melt curve stage may be added to both two-step and one-step RT-qPCR reaction: hold at 95° C. for 15 sec, hold at 60° C. for 15 sec then 95° C. for 15 sec.

In some embodiments, the single or multiplex RT-qPCR assay may use a commercially available PCR kit such as TaqPath RT-PCR kit (Thermo Fisher) and may be used in conjunction with the TaqPath 1-step master mix (Thermo Fisher). For example, RT-qPCR reactions may comprise 5 uL template+5 μL of reaction mix (2.5 uL TaqPath 1-step master mix, 0.5 uL TaqPath primer/probe mix, 1.0 uL internal control, and 1.0 RNase-free water). The assay may be performed in 384-well reaction plates in a QuantStudio 7 system (Applied Biosciences). The RT-qPCR may be conducted using the standard mode, consisting of, for example, a hold stage at 25° C. for 2 min, 53° C. for 10 min, and 95° C. for 2 min, followed by 40 cycles of a PCR stage at 95° C. for 3 sec then 60° C. for 30 sec; with a 1.6° C./sec ramp up and ramp down rate.

One of ordinary skill in the art will recognize the temperatures, the length of time at such temperatures, and the number of cycles to which a polynucleotide sample (e.g., DNA, RNA) must be subject to effectuate amplification of polynucleotide for the different methods of using an apparatus of the invention, e.g., screening, identification, quantification, etc. For example, in a preferred embodiment, denaturing temperatures are between 90° C. and 95° C., annealing temperatures are between 55° C. and 65° C., and elongation temperatures are dependent on the polymerase chosen (e.g., the optimal elongation temperature is about 72° C. for Taq polymerase). Also, the artisan or ordinary skill will recognize that that “hot starts” often begin PCR amplification methods, and that a final incubation of a polynucleotide sample at 75° C. may optionally be added to the end of any amplification method. For example, although a typical cycling profile is ˜94° for 1 min., 600 for 1 min., 72° for 1 min. (a typical rule for a 72° C. elongation is 1 minute for each 1000 base pairs being amplified), etc., an artisan or ordinary skill will recognize that the duration of time a sample remains at a certain temperature is dependent on the volume of the reaction, the concentration of the polynucleotide, etc. An artisan of ordinary skill will recognize that shorter durations at each temperature may be sufficient. (See, for example, U.S. Pat. Pub. No. 2011/0189736). In some embodiments, a change in the expression level of a certain gene or an increase in the gene copy number may indicate the presence of SCC such as HNSCC.

In some embodiments, the copy number of a target nucleic acid relative to a reference value can be determined by any suitable means, e.g., by detecting fluorescence intensity at one or more selected points during the exponential phase of amplification of the target nucleic acid.

From the value obtained and a reference value, which can, but need not be determined in parallel, the relative copy number is calculated. One method entails the detection of more than one threshold cycle value (Ct) and determining an “area between the thresholds.”

In certain embodiments, a method for calculating relative copy number is the 2^{-ΔΔCt} method described in Livak, K., Schmittgen, T., Analysis of Relative Gene Expression Data Using Real-Time Quantitative PCR and the 2^{-ΔΔCt} Method (2001 December) 25(4):402-8. If relative copy number is determined using the 2^{-ΔΔCt} method, the assay method can entail determining a threshold cycle (Ct) value for each sample or aliquot thereof. In particular embodiments, one can calculate Average and Delta Ct for each sample-assay group and the Standard Error of the Mean (SEM=s/sqrt(n)). The difference between the Ct value for the target sequence and the Ct value for the internal control sequence (ΔCt value) for each of the test and reference samples can then be determined. Then, the difference between the ΔCt value for the test sample and the ΔCt value for the reference sample (ΔΔCt) can be determined. One can then calculate the ΔΔCt error (SEM_{ΔΔCt}), the sum in quadrature of the SEM for the test and reference samples in the target and internal control assays, as described in Taylor, John R., An Introduction to Error Analysis, University Science Books, 1982, p. 56, which is incorporated by reference herein for this description. The copy number for the target sequence in the test sample relative to the reference sample can then be calculated, for example, according to the following formula: RCN=2^{-ΔΔCt±1.96*SEMΔΔCt}, where a factor of 1.96 is multiplied with SEM_{ΔΔCt} to reflect the 95% confidence interval for RCN.

To determine relative copy number using the 2^{-ΔΔCt} method in a multiplex format, the method can entail determining a threshold cycle (Ct) value for the target and internal control sequences in each sample, or aliquot thereof, and calculating relative copy number.

If relative copy number is determined using the 2^{-ΔΔCt} method for a plurality of target nucleic acid sequences from a single chromosome, the method can entail determining a threshold cycle (Ct) value for each target and relative copy number calculated as described above. This yields multiple relative copy numbers, one per target. If desired, a relative copy number can be calculated for the chromosome by taking the mean, geometric mean, or the like, of the calculated RCNs for the target nucleic acids from the chromosome or by pooling the Ct data between different target nucleic acids on the chromosome, if the amplification efficiencies and Ct values are similar between the target nucleic acids. For example, the data may be averaged across the plurality of preamplification replicates, and/or averaged across a plurality of amplification replicates (e.g., across multiple lanes and/or multiple columns of a matrix-type microfluidic device), and/or averaged across a plurality of targets on a chromosome. See, for example, Livak, et al., Methods. (2001 December) 25(4):402-8.

Accordingly, some embodiments comprise measuring a fold change in gene copy number of the one or more target polynucleotides, wherein the one or more target polynucleotides comprise one or more of human TP53, CDKN2A, CCND1, EGFR, PIK3CA, HRAS, KRAS, NRAS, and FBXW7, wherein an increase in the gene copy number of the one or more target polynucleotides compared to a wild-type gene copy number of the one or more of human p53, CDKN2A, CCND1, EGFR, PIK3CA, HRAS, KRAS, NRAS, and FBXW7 indicates the presence of SCC. In other embodiments, a decrease in the gene copy number of the one or more target polynucleotides compared to a wild-type gene copy number of the one or more of human p53, CDKN2A, CCND1, EGFR, PIK3CA, HRAS, KRAS, NRAS, and FBXW7 indicates the presence of SCC. In some embodiments, a deletion in a gene of one or more of human TP53, CDKN2A, CCND1, EGFR, PIK3CA, HRAS, KRAS, NRAS, and FBXW7 indicates the presence of SCC.

In some embodiments, relative expression of PCR products may be determined using the ΔΔCT method. Briefly, each set of samples was normalized using the difference in threshold cycle (CT) between the target gene and housekeeping gene (e.g., 18S, RNAaseP, beta-actin, or other gene used a control reference): ΔCT=(CT target gene-CT beta actin). Relative mRNA levels were calculated by the expression 2^{-ΔΔCT}, where ΔΔCT=ΔCT sample-ΔCT calibrator. Data analysis may be performed, for example, using Design and Analysis software.

In some embodiments, a fold change in expression of CDKN2A may be measured according to the steps of determining a change in Ct (ΔCt) value for the CDKN2A and for one of the 18S or the beta-actin for both the subject and a reference subject known to be SCC free; calculating the difference in ΔCt value (ΔΔCt) of the subject and the reference subject known to be SCC free obtained from the determining step; and computing a 2^{-ΔΔCt} to produce a final value, wherein the final value equals the fold change in expression of the CDKN2A gene, and wherein the fold change in expression of more than 5× relative to the 18S or the beta-actin indicates the presence of SCC in the biological sample.

In other embodiments, the fold change in expression of the target polynucleotide of 2 or greater, 3 or greater, 4 or greater, 5 or greater, 6 or greater, 7 or greater, 8 or greater, 9 or greater, or 10 or greater indicates the presence of SCC in the biological sample.

In some embodiments, the target polynucleotide is CDNK2A and at least one of a second target polynucleotide is a housekeeper gene, wherein 2^{-ΔΔCt} values for the CDNK2A and one of the housekeeper genes, wherein the housekeeper gene is one of 18S, RNAaseP, or beta-actin are determined, wherein a change in expression of the CDNK2A gene of more than 1×, 2×, 3×, 4×, or 5× relative to the second target polynucleotide indicates a presence of SCC.

Some embodiments of the disclosure comprise measuring a fold change of expression one or more target polynucleotides comprising measuring a change in Ct (ΔCt) value for each of the one or more target polynucleotides and a second polynucleotide, wherein the second polynucleotide comprises one or more of RNAseP, 18S, and beta-actin for both the subject and a reference subject known to be SCC free; calculating the difference in ΔCt value (ΔΔCt) of the subject and the reference subject known to be SCC free obtained from the measuring step; computing a 2^{-ΔΔCt} to produce a final value for each of the one or more target polynucleotides and the second polynucleotide; and comparing the final value for each of the one or more target polynucleotides to the second polynucleotide to form a comparison ratio, wherein a comparison ratio of 1 or greater indicates the presence of SCC and a comparison ratio of less than 1 indicates the absence of SCC in the biological sample.

In other embodiments, the comparison value of target polynucleotide to the second target polynucleotide that indicates the presences of cancer may be 2:1 or greater, 3:1 or greater, 4:1 or greater, 5:1 or greater, 6:1 or greater, 7:1 or greater, 8:1 or greater, 9:1 or greater, or 10:1 or greater.

In other embodiments, a method of detecting Human papilloma Virus (HPV) mediated Squamous Cell Carcinoma (SCC) comprising: extracting polynucleotides from a saliva sample from a subject; amplifying the extracted polynucleotides in the saliva sample using a polymerase chain reaction (PCR) method; and detecting target polynucleotides in the amplified polynucleotides, wherein the target polynucleotides comprise one or more of an E6 gene of HPV16, an E7 gene of HPV16, and an E7 gene of HPV18; wherein detection of the target polynucleotides indicates a presence or absence of HPV mediated SCC in the biological sample. In some embodiments, the SCC is head and neck Squamous Cell Carcinoma (HNSCC).

In some embodiments, a method of detecting non-Human Papilloma Virus mediated squamous cell carcinoma (SCC) comprises: extracting polynucleotides from a saliva sample from a subject; amplifying the polynucleotides in the saliva sample using a polymerase chain reaction (PCR) method; and detecting target polynucleotides in the amplified polynucleotides, wherein the target polynucleotides comprise one or more of TP53, CDKN2A, CCND1, EGFR, PIK3CA, HRAS, KRAS, NRAS, and FBXW7; wherein a change in expression level of the target polynucleotides or a change in copy number of the target polynucleotides as compared to a biological sample from a reference subject known to be SCC free indicates a presence or absence of non-HPV mediated SCC in the biological sample. In some embodiments, the SCC is head and neck Squamous Cell Carcinoma (HNSCC).

In some embodiments, a method of detecting non-HPV mediates SCC further comprises measuring a change in Ct (ΔCt) value for each of the one or more target polynucleotides and a second polynucleotide, wherein the second polynucleotide comprises one or more of RNAseP, 18S, and beta-actin for both the subject and a reference subject known to be SCC free; calculating the difference in ΔCt value (ΔΔCt) of the subject and the reference subject known to be SCC free obtained from the measuring step; computing a 2^{-ΔΔCt} to produce a final value for each of the one or more target polynucleotides and the second polynucleotide; and comparing the final value for each of the one or more target polynucleotides to the second polynucleotide to form a comparison ratio, wherein the comparison ratio indicates a fold change in expression of each of the one or more target polynucleotides, and wherein a comparison ratio greater than 1 indicates the presence of SCC and a comparison ratio of less than 1 indicates the absence of SCC in the biological sample. In some embodiments, the SCC is head and neck Squamous Cell Carcinoma (HNSCC).

In some embodiments, a method of method of detecting non-HPV mediates SCC comprises measuring a fold change in gene copy number of the one or more target polynucleotides, wherein an increase in the gene copy number of the one or more target polynucleotides as compared to a wild-type gene copy number of one or more of human p53, CDKN2A, CCND1, EGFR, PIK3CA, HRAS, KRAS, NRAS, and FBXW7 indicates the presence of SCC. In some embodiments, a decrease in copy number may indicate the presence of SCC. In some embodiments, the SCC is head and neck Squamous Cell Carcinoma (HNSCC).

In some embodiments, a method of detecting non-Human Papilloma Virus mediated squamous cell carcinoma comprising: extracting polynucleotides from a saliva sample from a subject; subjecting the extracted polynucleotides to conditions that amplify the extracted polynucleotides using a polymerase chain reaction (PCR) method; detecting one or more target polynucleotides in the amplified extracted polynucleotides, wherein the one or more target polynucleotides comprise one or more of p53, CDKN2A, CCND1, EGFR, PIK3CA, HRAS, KRAS, NRAS, and FBXW7; measuring a change in Ct (ΔCt) value for each of the one or more target polynucleotides and a second polynucleotide, wherein the second polynucleotide comprises one or more of RNAseP, 18S, and beta-actin for both the subject and a reference subject known to be SCC free; calculating the difference in ΔCt value (ΔΔCt) of the subject and the reference subject known to be SCC free obtained from the measuring step; computing a 2^{-ΔΔCt} to produce a final value for each of the one or more target polynucleotides and the second polynucleotide; comparing the final value for each of the one or more target polynucleotides to the second polynucleotide to form a comparison ratio, wherein a comparison ratio greater than 1 indicates the presence of SCC and a comparison ratio of less than 1 indicates the absence of SCC in the biological sample. In some embodiments, the SCC is head and neck Squamous Cell Carcinoma (HNSCC).

In some embodiments, the determination of the presence or absence of a certain cancer, or of a certain result from the detection of the target polynucleotides may be confirmed or corroborated using immunohistochemical staining.

In some embodiments, certain steps of the method, such as the mixing, contacting, extracting polynucleotides, and subjecting/amplification steps may be partially or fully automated. In some embodiments, all the steps of the methods described herein may be partially or fully automated.

This disclosure also provides a kit for the detection of one or more target polynucleotides present in a biological sample using polymerase chain reaction assay. An exemplary kit may include one or more primer pairs such that the primer pair can detect and/or amplify target polynucleotides, if present, in the sample. In some embodiments, the target polynucleotide may include a polynucleotide from one or more HPV strains such as HPV16 and/or HPV18. In some embodiments, the target polynucleotide comprises one or more of the E6 and E7 gene of the HPV16 strain and E7 of the HPV18 strain. In some embodiments, the target polynucleotides comprise HPV genes and human genes such as one or more of human p53, CDKN2A, CCND1, EGFR, PIK3CA, HRAS, KRAS, NRAS, and FBXW7. In some embodiments, the target polynucleotides also may include one or more of RNAseP, 18S, and beta-actin. In one embodiment, the target polynucleotide is CDKN2A. Preferably, the PCR assay is RT-qPCR. Amplification products may be detected using methods that are well known to a person of ordinary skill in the art. Exemplary primer pairs and target sequences are listed in Tables II-VI.

An exemplary kit also may include a buffering agent such as TE or TBE, one or more non-ionic detergents such as a polysorbate (e.g., polysorbate-20, polysorbate-80), optionally one or more sample additives, one or more polymerase (e.g., DNA polymerase, reverse transcriptase), nucleotides/nucleosides, detecting agents, or any reagents for performing PCR, qPCR, or RT-qPCR, and one or more vial/containers to hold each component as well as to collect and process the saliva sample.

Preferably, the primer pairs that are supplied with the kit are provided in a lyophilized form. Preferably, an exemplary kit also may include DNA and/or RNA purification kits which are known in the art and commercially available, from, for example, Quiagen or ThermoFisher Scientific.

Another embodiment of a kit of the disclosure may comprise one or more collection tubes, at least one buffering agent, at least one non-ionic detergent, a plurality of RT-qPCR primers, one or more RT-qPCR reagents, and one or more polymerases.

In another embodiment, the primers of the kit are configured to amplify and/or detect polynucleotides from a subject having or suspected of having SCC such as HNSCC or a cervical cancer. In particular, at least one sequence of a target polynucleotide comprises one or more of the E6 and E7 gene of the HPV16 strain and E7 of the HPV18 strain and/or one or more of human genes such as one or more of human p53, CDKN2A, CCND1, EGFR, PIK3CA, HRAS, KRAS, NRAS, and FBXW7. In some embodiments, the target polynucleotides also may include one or more of RNAseP, 18S, and beta-actin. In some embodiment, the kits include instructions to carry out the methods described herein.

Exemplary primers that may be used with embodiments of the invention, including a kit for the detection of one or more target polynucleotides present in a biological sample, comprise one or more primer pairs listed in Table II-V.

### RESULTS AND DISCUSSION

This study aims to apply the UIUC protocol developed for SARS-CoV-2 detection to measure and quantify the presence of either HPV-mediated or non-HPV-mediated tumor DNA from saliva of patients diagnosed with head-and-neck squamous cell carcinoma (HNSCC), with the ultimate goal of improving the current standard diagnostic procedures for oropharyngeal squamous cell carcinoma (OSCC). Carle Foundation hospital and others currently employ immunohistochemistry of p16 (CDKN2a) for detection of HPV-mediated OSCC, which is caused by viral DNA insertion into the patient's genome, ultimately leading to uncontrolled growth and tumor formation.

To demonstrate the potential of this approach, we first sought to develop a method for screening HPV-mediated HNSCC. Of the nearly 200 genotypes of HPV, HPV16 is most commonly detected in oropharyngeal squamous cell carcinoma (OSCC), and meta-analysis suggests that 82% of all HPV-positive HNSCC are attributable to HPV16. HPV18, the second most prevalent type in OSCC, accounts for 5.9% of HNSCC cases worldwide and is also targeted by our PCR diagnostic assay. Our technology development process will proceed from optimization with spiked samples (appropriate HPV plasmid DNA into saliva), and then moving forward to clinical samples from patients from Carle Foundation Hospital.

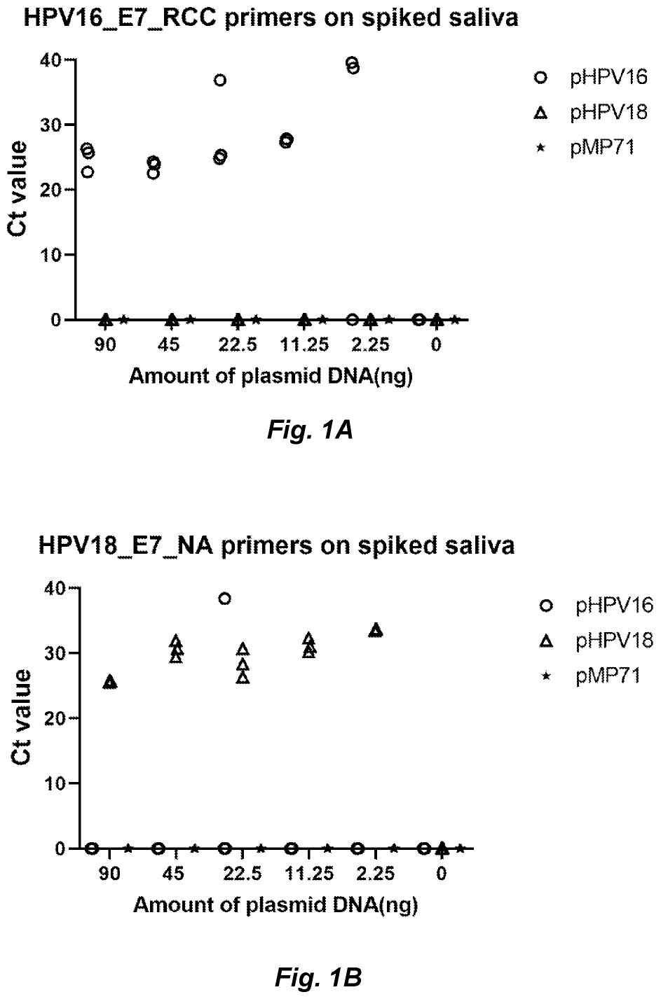

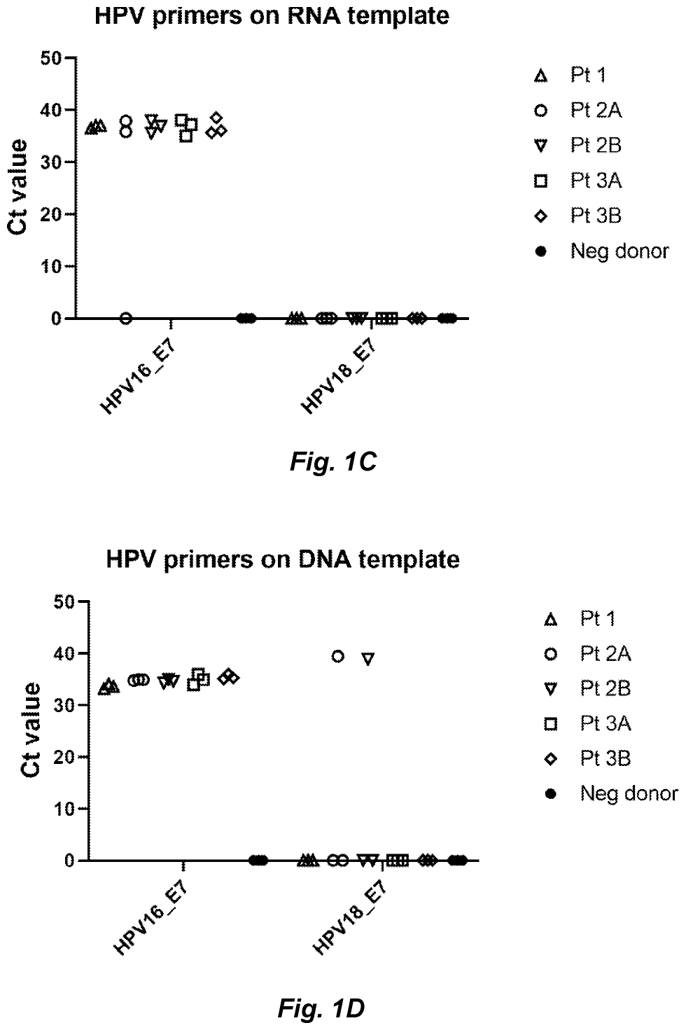

We purchased both pHPV16 and pHPV18 plasmid DNA from ATCC. Following manufacturer's protocol for plasmid expansion in competent E. coli cells, we purified large quantities of the two plasmid DNA constructs for downstream experiments. As a negative control, we also purified plasmid DNA from a retroviral plasmid pMP71. Screening for 6 reported HPV16 primer pairs and 1 HPV18 primer pair was performed by spiking known amounts of either pHPV16 or pHPV18 plasmid DNA in saliva from a healthy donor, heated at 95° C., cooled to room temperature, mixed 1:1 with 2×TBE buffer, and used as template for direct SYBR green qPCR. Primer pairs that formed amplicon in “H20 only” control were eliminated; those that had multiple melting curves (indicates non-specificity of primers towards target) were also eliminated. At the end of the screening and optimization process, we decided to go with primer pairs “HPV16_E7_RCC”, “HPV16_E6 NNK” and “HPV18_E7_NA” that target the HPV16 E7, HPV16 E6, and HPV18 E7 oncogene, respectively. Titration of spiked plasmid DNA in saliva was performed to establish sensitivity of primer pairs. Encouragingly, our dilution experiments with HPV16 and HPV18 plasmid DNA spiked into saliva showed that these genes could be detected very sensitively (down to ˜2 ng), and specifically (no detection of retroviral plasmid pMP71) (FIGS. 1A and 1B).

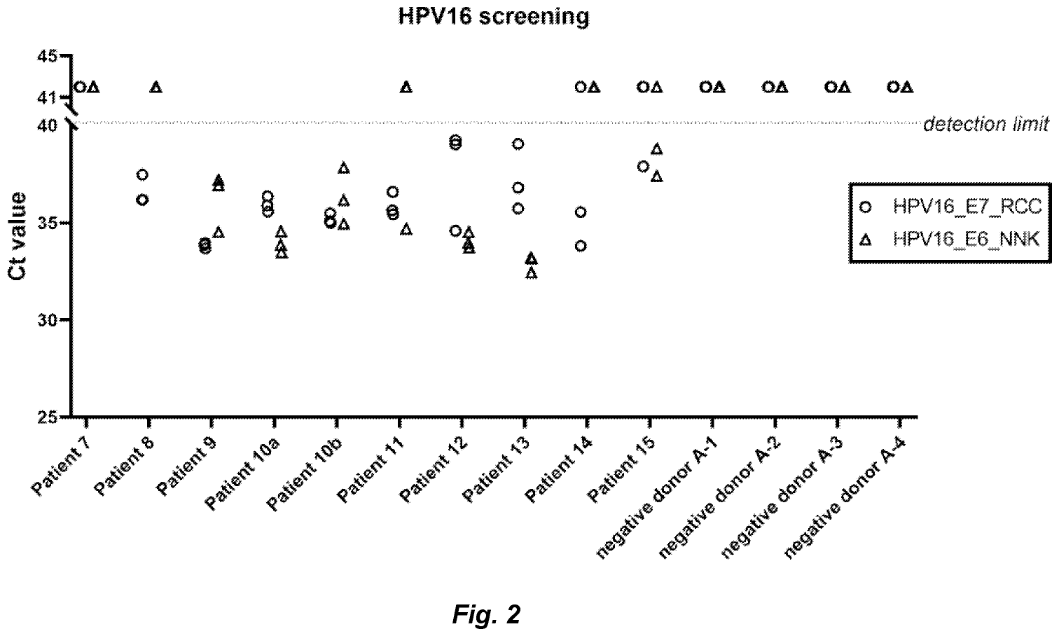

Saliva samples from HNSCC patients at Carle Foundation Hospital were used for validation of our studies (IRB No. 20CCC3279). Replicate samples were also received from some patients, designated as Pt 2A, Pt 2B, Pt 3A, Pt 3B, Pt 5A, Pt 5B, Pt10A, and Pt10B. We initially tested if our direct saliva to RT-qPCR approach using SYBR 1-Step kit protocol would work using our HPV16 and HPV18 primer pairs and the optimized PCR protocol described above. However, no HPV target genes were detected from the clinical samples using the direct approach. Next, we extracted total RNA from patient saliva samples using Qiagen RNEasy kit. Reverse transcription (RT) was performed using Invitrogen Superscript IV VILO Master Mix, following manufacturer's protocol. PCR was then performed using SYBR Green master mix. As shown in FIG. 1C, HPV16 E7 oncogene, but not HPV18 E7, was detected in all samples tested. Similar results were obtained when genomic DNA was extracted from patient saliva using Qiagen Dneasy tissue kit and used as template for qPCR using the SYBR Green master mix (FIG. 1D). To further improve the specificity of the assay, we added another HPV16 primer targeting the E6 oncogene (“HPV16_E6_NNK”) to the screening strategy. Using this approach, a sample would be positive for HPV16 if both E7 and E6 targets were detected in the RT-PCR reaction, as shown in FIG. 2 and summarized in Table I.

TABLE I
Screening for HPV16, HPV18, and CDKN2a in human saliva samples.

| | | | | | | RT-PCR |
|---|---|---|---|---|---|---|
| | | | | | | CDKN2a |
| | | | | | | result |
| | | | | | | [2-^{(ΔΔCt)} |
| | | | | | | values |
| | | | | | | rel to |
| | HPV16 | HPV16 | | | HPV18 | 18Sand |
| Sample | Primer 1 | Primer 2 | | HPV16 | result | negative |
| name | HPV16_E7_RCC | HPV16_E6_NNK | Score | result | HPV18_E7_NA | control] |
| Patient 1 | Positive | Positive | 2/2 | Positive | Positive | positive |
| Patient 2A | Positive | Negative | 1/2 | Negative | Negative | negative |
| Patient 2B | Positive | Negative | 1/2 | Negative | Negative | negative |
| Patient 3A | Positive | Positive | 2/2 | Positive | Negative | negative |
| Patient 3B | Positive | Positive | 2/2 | Positive | Negative | negative |
| Patient 4 | Negative | Negative | 2/2 | Negative | Negative | negative |
| Patient 5A | Positive | Positive | 2/2 | Positive | Negative | positive |
| Patient 5B | Positive | Positive | 2/2 | Positive | Negative | positive |
| Patient 6 | Positive | Positive | 2/2 | Positive | Negative | negative |
| Patient 7 | Negative | Negative | 0/2 | Negative | Negative | negative |
| Patient 8 | Positive | Negative | 1/2 | Negative | Negative | negative |
| Patient 9 | Positive | Positive | 2/2 | Positive | Negative | positive |
| Patient 10a | Positive | Positive | 2/2 | Positive | Negative | positive |
| Patient 10b | Positive | Positive | 2/2 | Positive | Negative | positive |
| Patient 11 | Positive | Negative | 1/2 | Negative | Negative | negative |
| Patient 12 | Positive | Positive | 2/2 | Positive | Negative | positive |
| Patient 13 | Positive | Positive | 2/2 | Positive | Positive | positive |
| Patient 14 | Negative | Negative | 0/2 | Negative | Negative | negative |
| Patient 15 | Negative | Negative | 0/2 | Negative | Negative | negative |
| Patient 16 | Positive | Positive | 2/2 | Positive | Negative | positive |
| Patent 17 | Positive | Negative | 1/2 | Negative | Negative | positive |
| Patient 18 | Negative | Negative | 2/2 | Negative | Negative | negative |
| Negative | Negative | Negative | 0/2 | Negative | Negative | |
| donor A-1 | | | | | | |
| Negative | Negative | Negative | 0/2 | Negative | Negative | |
| donor A-2 | | | | | | |
| Negative | Negative | Negative | 0/2 | Negative | Negative | |
| donor A-3 | | | | | | |
| Negative | Negative | Negative | 0/2 | Negative | Negative | |
| donor A-4 | | | | | | |

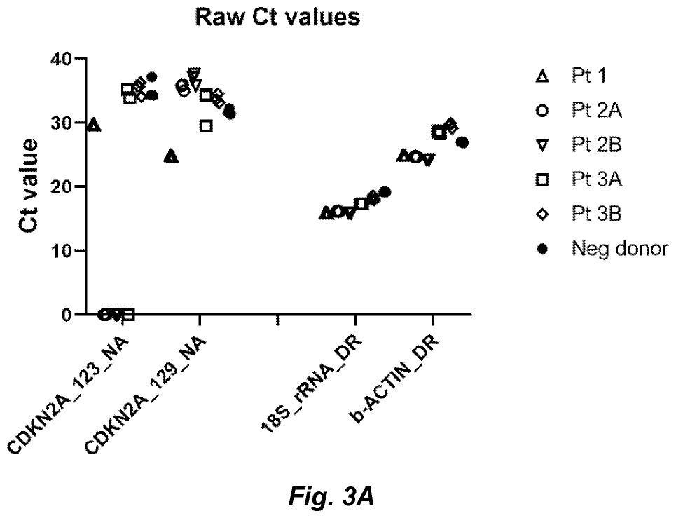

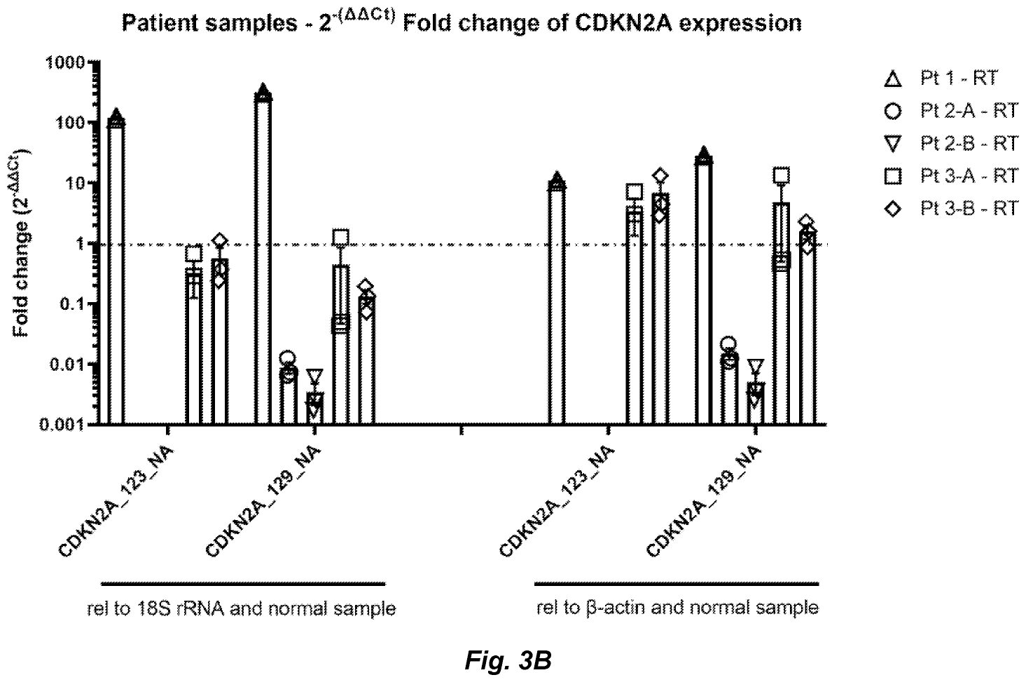

HPV-positive HNSCC exhibits genetic alterations that are caused by the HPV oncoprotein E7, which directly binds to the tumor suppressor Rb, resulting in the overexpression of the tumor suppressor gene p16^{INK4A} (CDKN2A). The current standard diagnostic surrogate marker for HPV infection in OSCC at Carle Foundation Hospital (and in most hospitals) is immunohistochemical analysis of p16^{INK4A} We therefore sought to develop a protocol that could detect for both the presence of the HPV oncogene insertion as well as changes to the expression of CDKN2A. To this end we screened a total of 4 CDKN2A primer pairs and 6 primer pairs that target housekeeping genes, using the PCR parameters that were optimized for the HPV16 E7 and E6 primer pairs. To select for the best primer pairs, we tested total RNA extracted from various sources such as brain, placenta, human colorectal cancer cell line HCT116 and murine glioma cell line GL261, as well as saliva from 3 patients and 1 negative donor. Once again, primer pairs that formed amplicon in “H20 only” control were eliminated; those that had multiple melting curves (indicates non-specificity of primers towards target) were also eliminated. Using our RT-PCR approach with two primer pairs that target CDKN2A and housekeeping genes 18S rRNA and β-actin (FIG. 3A), we demonstrate CDKN2A up-regulation in 6 out of 15 HNSCC patients after 2^{-(ΔΔCt)} values were calculated relative to either the housekeeping gene 18S rRNA or β-actin, and the HPV-negative saliva sample (FIG. 3B, last column of Table I). The results of this test will be compared to that of the immunohistochemistry results for CDKN2A as soon as data is shared to us from the Carle Foundation Hospital.

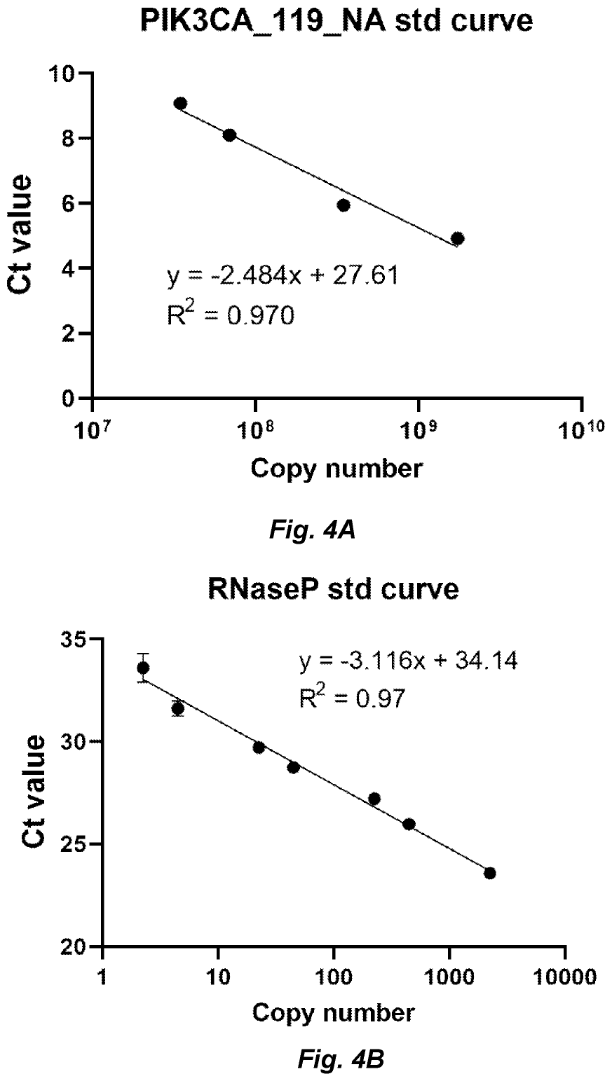

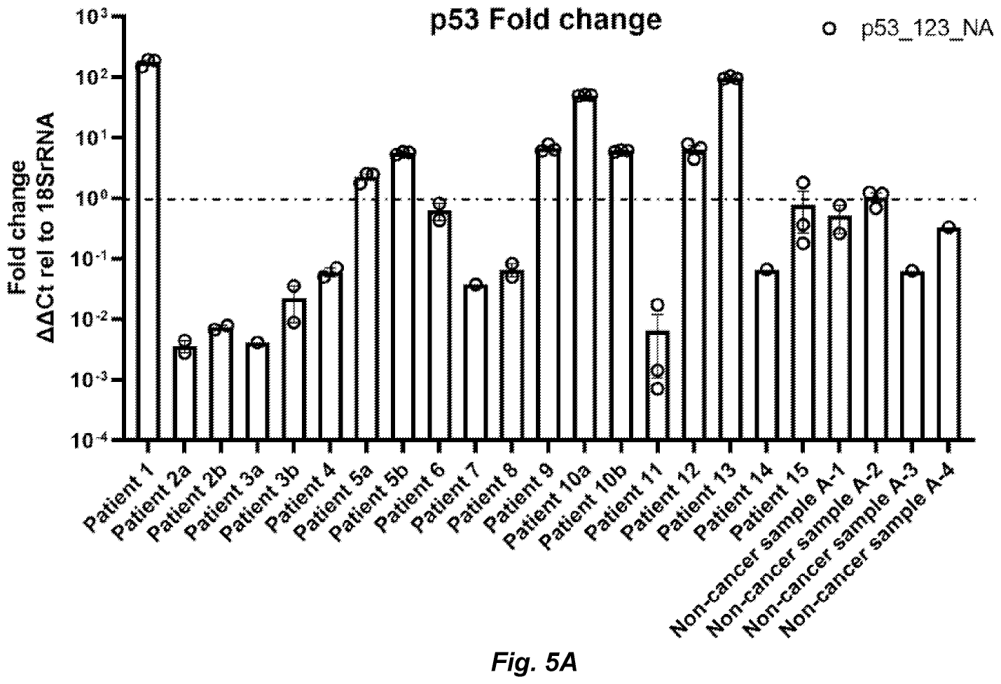

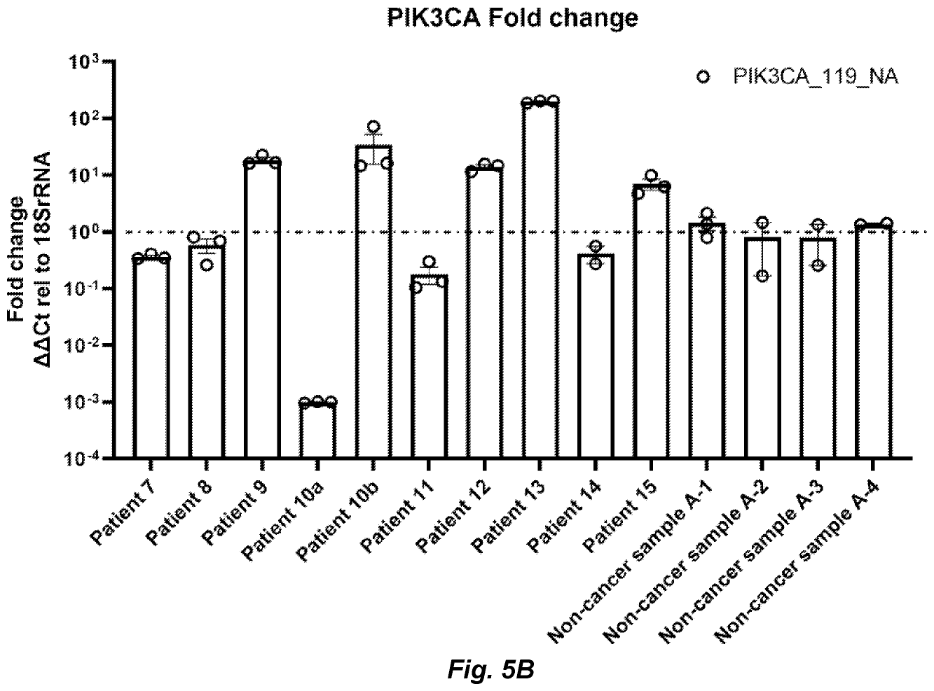

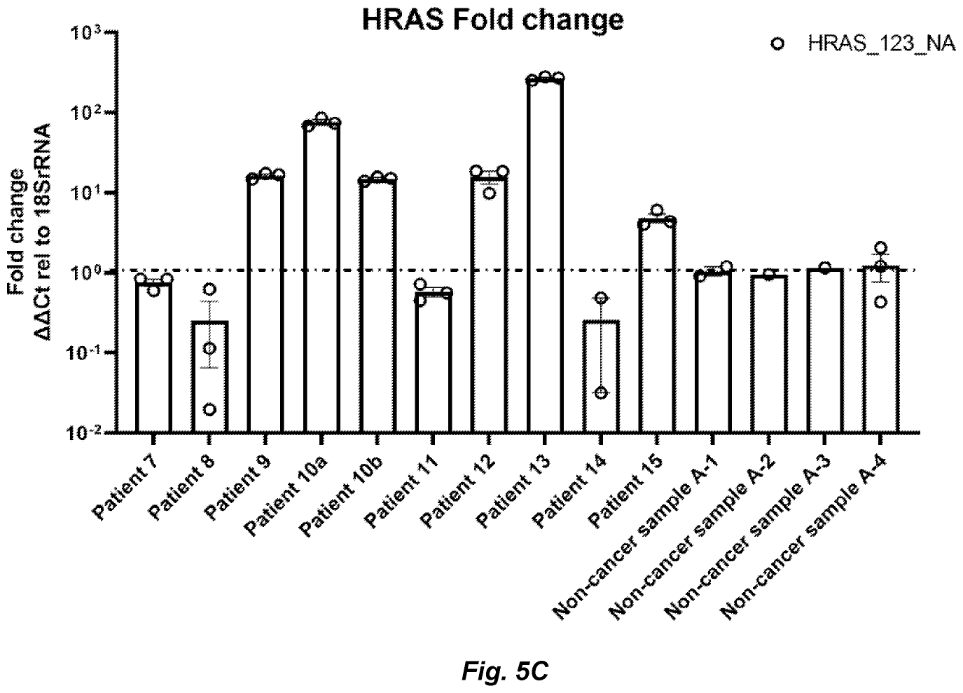

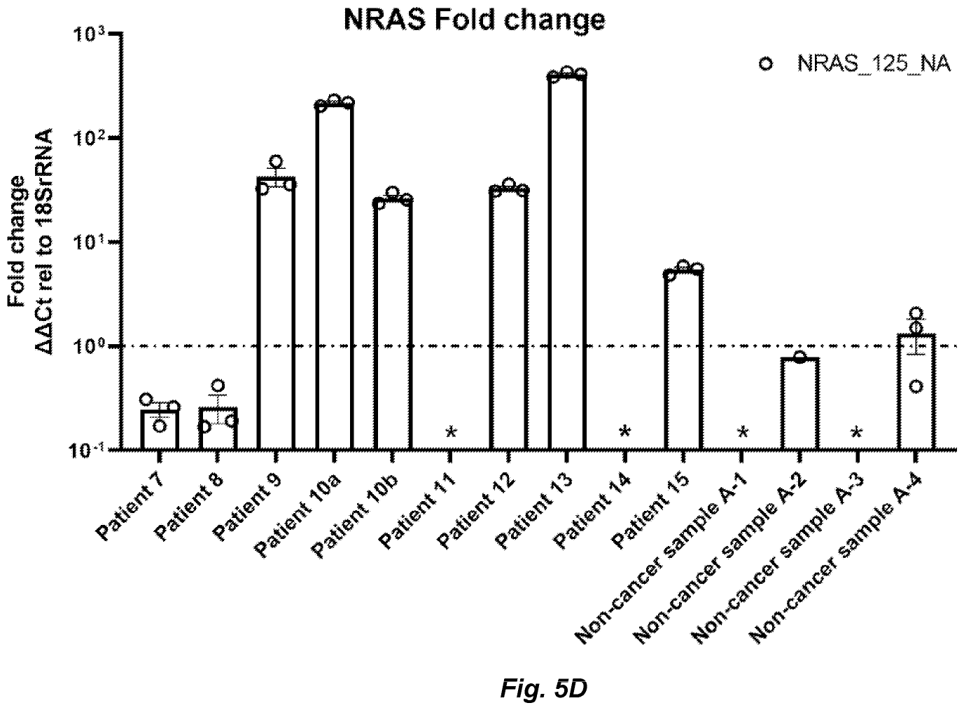

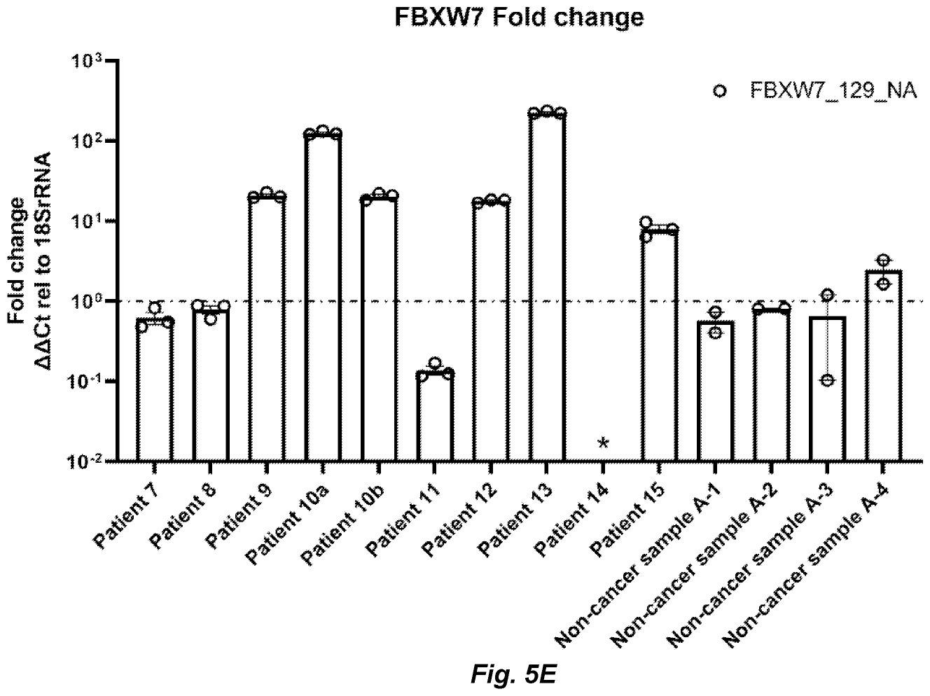

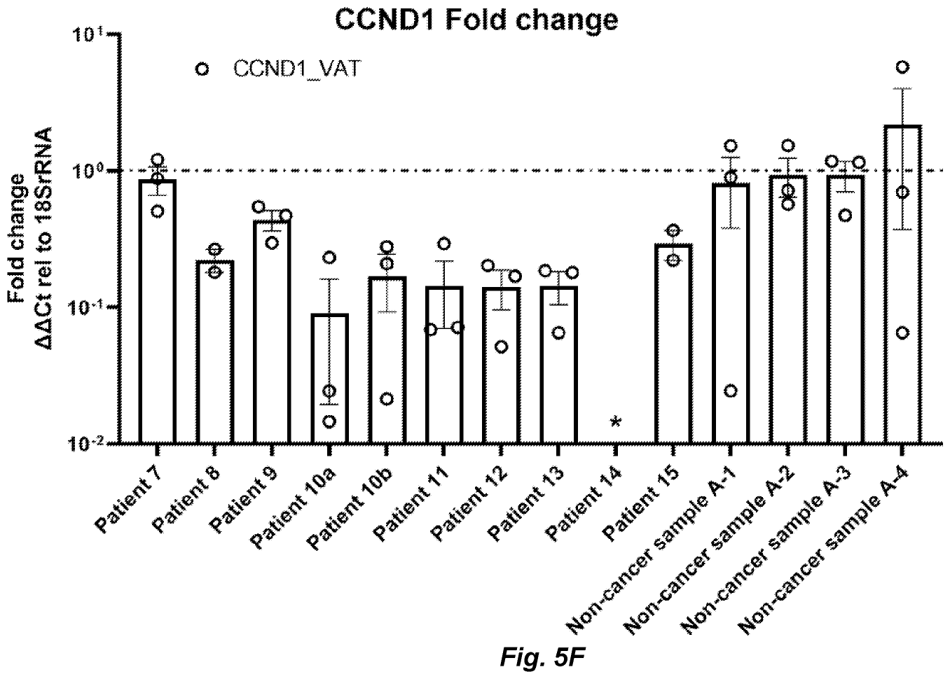

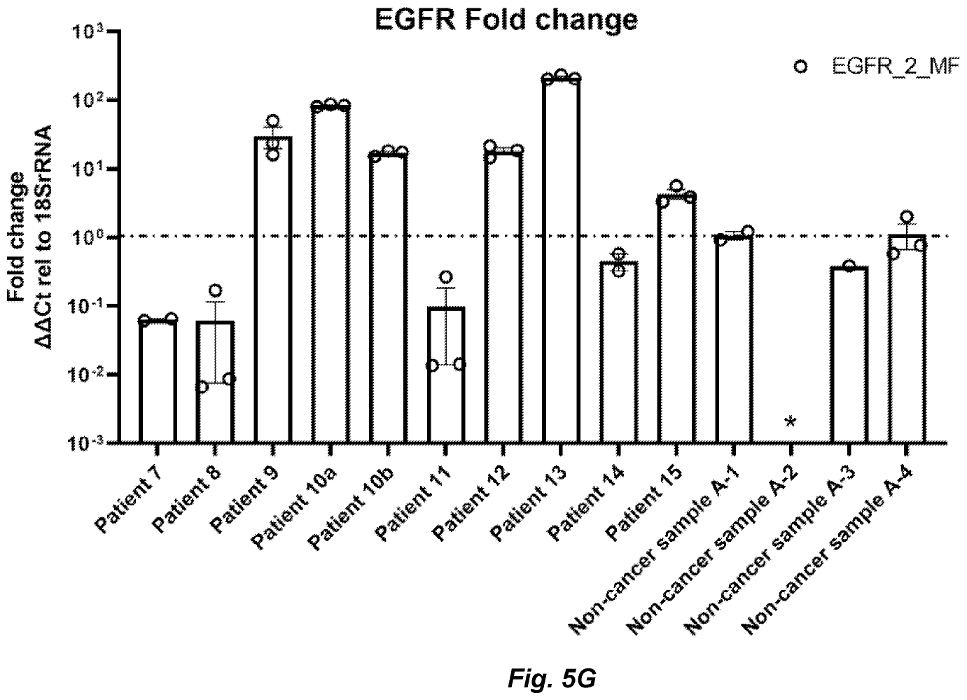

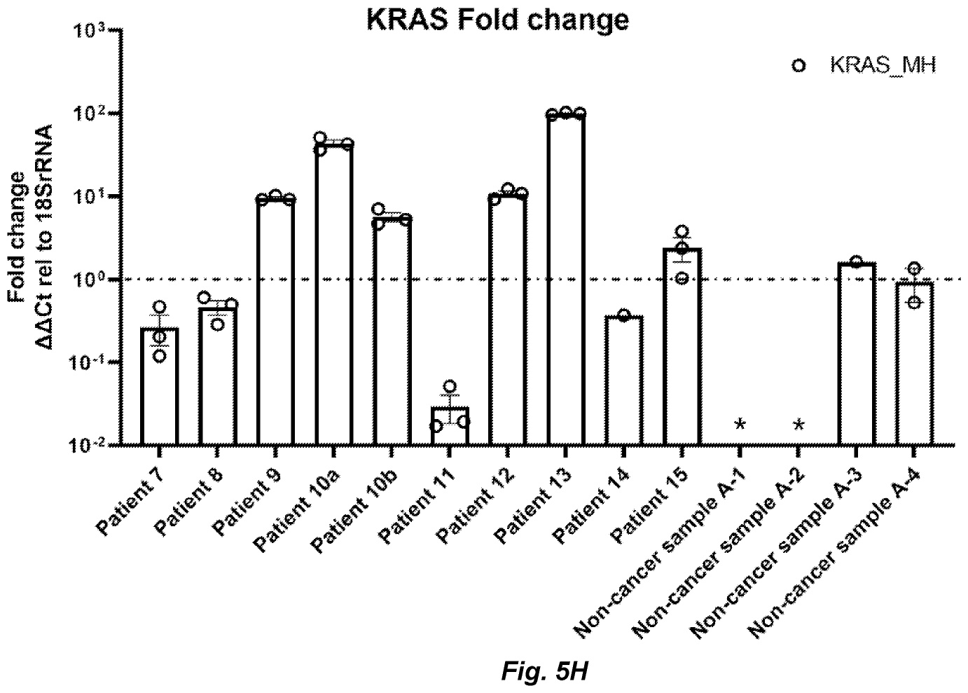

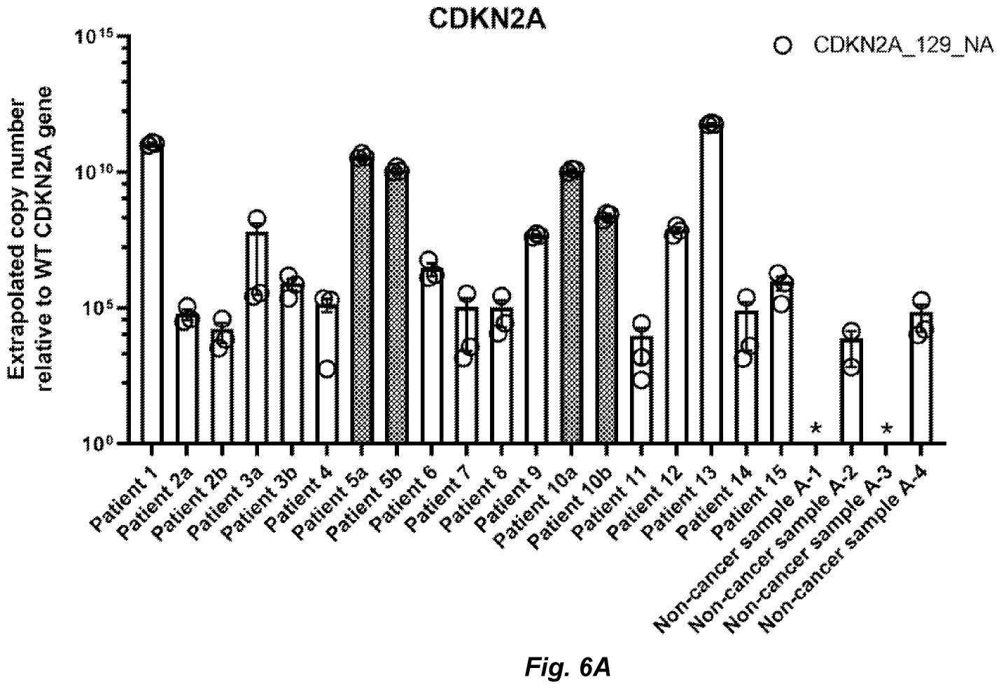

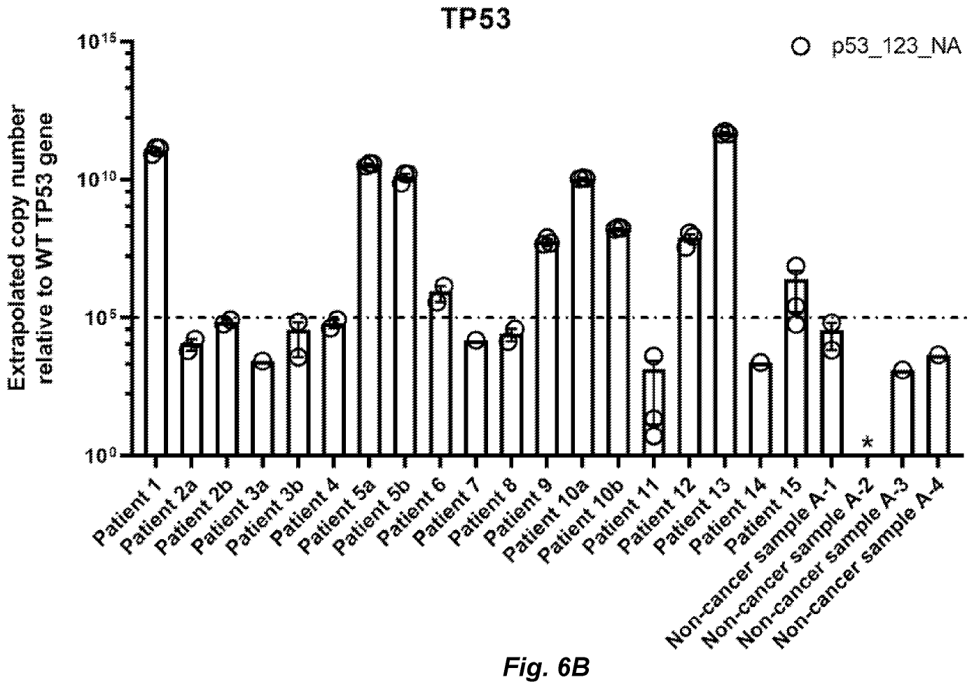

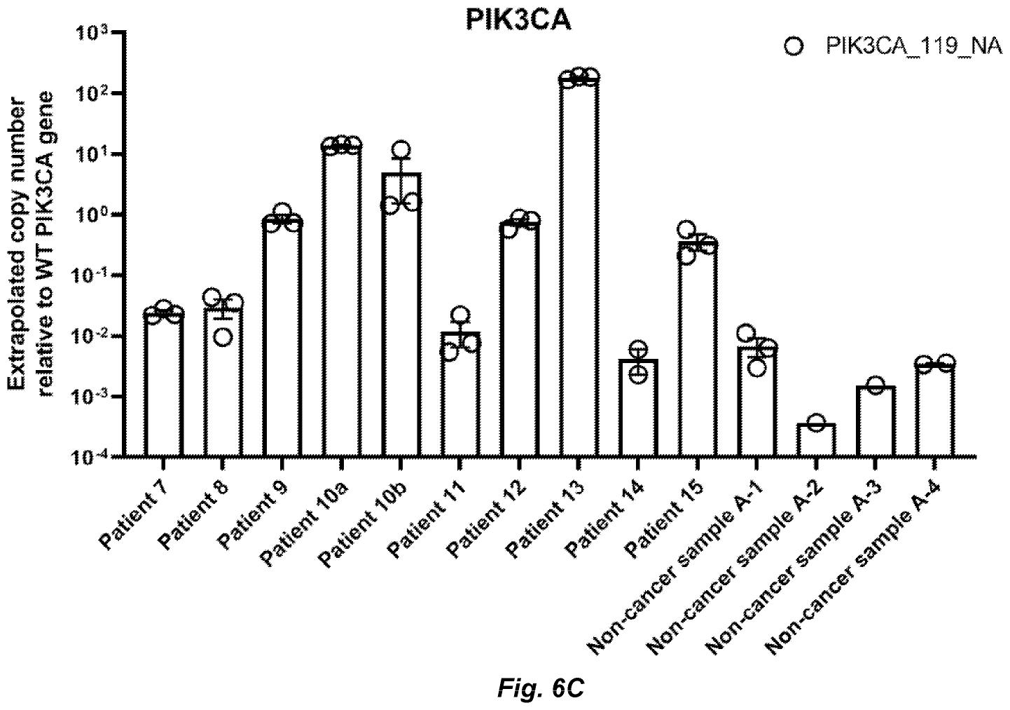

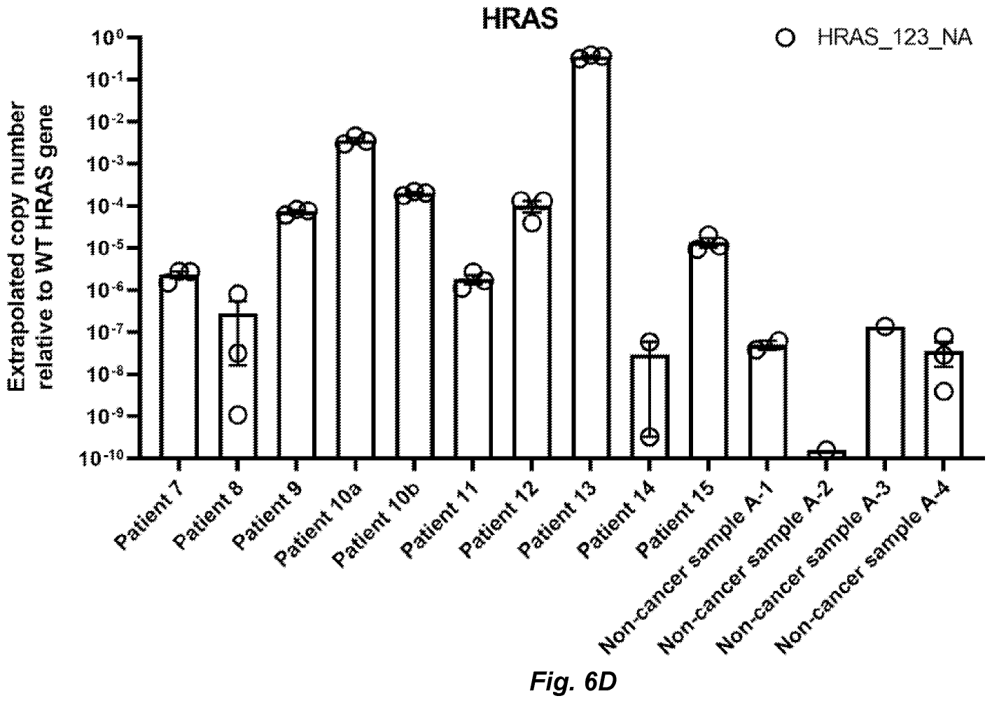

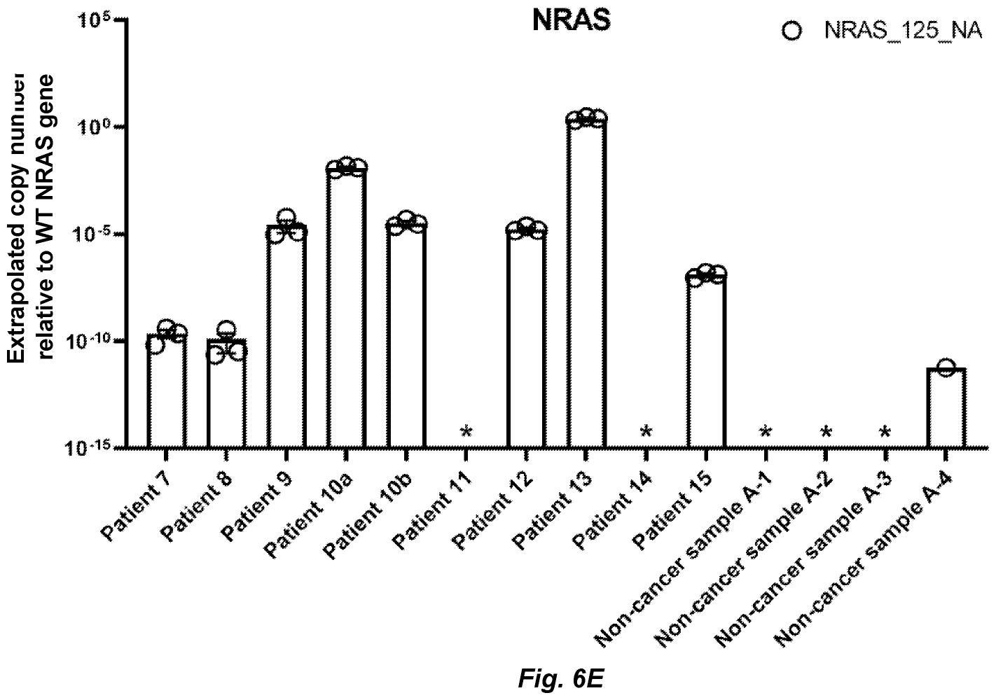

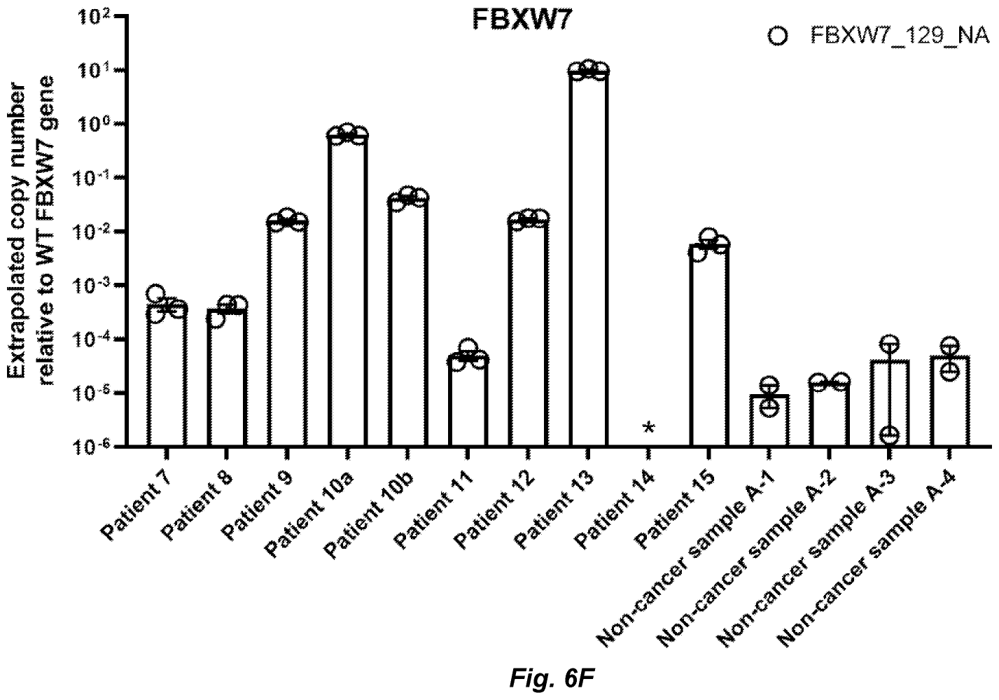

For non-HPV-mediated HNSCC, we will be screening for other tumor biomarkers that have been reported in large scale studies of HNSCC to exhibit changes either in expression level or mutations brought about by insertions/deletions (INDELs). Reported primers targeting TP53, CCND1, EGFR, PIK3CA, HRAS, KRAS, NRAS, and FBXW7 were screened on total RNA extracted from HNSCC patient samples and healthy donors using the optimized 2-step RT-PCR conditions optimized for HPV-screening. To further validate candidate primers for each target gene, we purchased plasmid DNA containing wild-type versions of CDKN2A, TP53, CCND1, EGFR, PIK3CA, HRAS, KRAS, NRAS, and FBXW7 from Addgene. A standard curve (FIG. 4) is generated from each plasmid DNA construct for extrapolation of copy number in unknown samples. The list of primers that have been validated using this approach are listed on Table II. The analysis and interpretation of results will depend on whether the gene target is up- or down-regulated more than two-fold (calculated using 2^{-(ΔΔCt)} method; FIG. 5) or exhibited increase in gene copy number in HNSCC patient saliva samples as extrapolated from our standard curves (FIG. 6 and Table III). In the latter approach, it is important to emphasize that the primers were tested against the wild-type version of their respective target genes. Reported primers targeting CCND1, EGFR, and KRAS did not bind to the reference pDONR223_CCND1_WT, pDONR223_EGFR_WT, and pDONR223_KRAS_WT plasmids, respectively, even though the same set of primers generated Ct values when tested on purified total RNA from patient saliva samples. One possible approach is to acquire tissue sections from the patients' biopsy sample for immune-histochemical staining using antibodies specific for our targets.

TABLE II
Validated primers for non-HPV-mediated HNSCC.

| Target | | | |
|---|---|---|---|
| Gene | Primer name | | Sequence |
| CDKN2A | CDKN2A_129_NA | Forward | 5′-ACAAATTCTCAGATCATCAGTCCTC-3′ |
| | | | (SEQ ID NO: 17) |
| | | Reverse | 5′-AGGAGCTGGGCCATCG-3′ (SEQ ID NO: |
| | | | 18) |
| CDKN2A | CDKN2A_123_NA | Forward | 5′-GGGTCGGGTGAGAGTGG-3′ (SEQ ID NO: |
| | | | 19) |
| | | Reverse | 5′-TGGCTCTGACCATTCTGTTCT-3′ (SEQ ID |
| | | | NO: 20) |
| TP53 | p53_123_NA | Forward | 5′-TCATCTTGGGCCTGTGTTATC-3′ (SEQ ID |
| | | | NO: 21 ) |
| | | Reverse | 5′-TGTGATGATGGTGAGGATGG-3′ (SEQ ID |
| | | | NO: 22) |
| TP53 | p53_127_NA | Forward | 5′-TGGCTCTGACTGTACCACCATC-3′ (SEQ |
| | | | ID NO: 23) |
| | | Reverse | 5′-GTGGCAAGTGGCTCCTGA-3′ (SEQ ID |
| | | | NO: 24) |
| PIK3CA | PIK3CA_119_NA | Forward | 5′-CCCCCTCCATCAACTTCTTC-3′ (SEQ ID |
| | | | NO: 25) |
| | | Reverse | 5′-GAAAAAGCCGAAGGTCACAA-3′ (SEQ ID |
| | | | NO: 26) |
| PIK3CA | PIK3CA_129_NA | Forward | 5′-TTTGATGACATTGCATACATTCG-3′ (SEQ |
| | | | ID NO: 27) |
| | | Reverse | 5′-GATCCAATCCATTTTTGTTGTCCAG-3′ |
| | | | (SEQ ID NO: 28) |
| HRAS | HRAS_123_NA | Forward | 5′-GTGGTCATTGATGGGGAGAC-3′ (SEQ ID |
| | | | NO: 29) |
| | | Reverse | 5′-GATGGCAAACACACACAGGA-3′ (SEQ ID |
| | | | NO: 30) |
| HRAS | HRAS_2_HK | Forward | 5′-GACGGAATATAAGCTGGTGG-3′ (SEQ ID |
| | | | NO: 31) |
| | | Reverse | 5′-AGGCACGTCTCCCCATCAAT-3′ (SEQ ID |
| | | | NO: 32) |
| NRAS | NRAS_125_NA | Forward | 5′-ACACCCCCAGGATTCTTACAG-3′ (SEQ ID |
| | | | NO: 33) |
| | | Reverse | 5′-CGCCTGTCCTCATGTATTGG-3′ (SEQ ID |
| | | | NO: 34) |
| NRAS | NRAS_122_NA | Forward | 5′-GATGTGGCTCGCCAATTAAC-3′ (SEQ ID |
| | | | NO: 35) |
| | | Reverse | 5′-GATTGTCAGTGCGCTTTTCC-3′ (SEQ ID |
| | | | NO: 36) |
| | | Reverse | 5′-TCAAAGAATGGTCCTGGACC-3′ (SEQ ID |
| | | | NO: 37) |
| KRAS | KRAS_MH | Forward | 5′-ATGACTGAATATAAACTTGTGGTAG-3′ |
| | | | (SEQ ID NO: 38) |
| | | Reverse | 5′-TTACATAATTACACACTTTGTCTTTGAC- |
| | | | 3′ (SEQ ID NO: 39 ) |
| FBXW7 | FBXW7_129_NA | Forward | 5′-TTGAGACAGGCCAGTGTTTACAT-3′ (SEQ |
| | | | ID NO: 40) |
| | | Reverse | 5′-CAGTCTCTGGATCCCACACC-3′ (SEQ ID |
| | | | NO: 41) |
| CCND1 | CCND1_PEP | Forward | 5′-GCAGAAGGAGGTCCTGCCGT-3′ (SEQ ID |
| | | | NO: 42) |
| | | Reverse | 5′-GTCACACTTGATCACTCTGG-3′ (SEQ ID |
| | | | NO: 43) |
| CCND1 | CCND1_VAT | Forward | 5′-CCGTCCATGCGGAAGATC-3′ (SEQ ID |
| | | | NO: 44 ) |
| | | Reverse | 5′-GAAGACCTCCTCCTCGCACT-3′ (SEQ ID |
| | | | NO: 45) |
| EGFR | EGFR_2_MF | Forward | 5′-ATGCAAATAAAACCGGACTGA-3′ (SEQ |
| | EGFR_MS | | ID NO: 46) |
| | | Reverse | 5′-CCAGCCTCTCACCCTGTAAA-3′ (SEQ ID |
| | | | NO: 47) |
| EGFR | EGFR_JX | Forward | 5′-TGACTGAGGACAGCATAGACGA-3′ (SEQ |
| | | | ID NO: 48 ) |
| | | Reverse | 5′-GGGCTGGACAGTGTTGAGATAC-3′ (SEQ |
| | | | ID NO: 49) |
| EGFR | EGFR_MS | Forward | 5′-CATGTCGATGGACTTCCAGA-3′ (SEQ ID |
| | | | NO: 50) |
| | | Reverse | 5′-GGGACAGCTTGGATCACACT-3′ (SEQ ID |
| | | | NO: 51) |
| RNase P | RP_CDC | Forward | 5′-AGATTTGGACCTGCGAGCG-3′ (SEQ ID |
| | | | NO: 52) |
| | | Reverse | 5′-GAGCGGCTGTCTCCACAAGT-3′ (SEQ ID |
| | | | NO: 53) |

The primer pairs listed in Table II are described, for example, in Wang et al., Sci Transl Med. 2015 Jun. 24; 7 (293):293ra104; Kiaris et al., Int J Oncol 7, 75-80, (1995); Hoa et al., Cancer research 62, 7154-7156 (2002); Peghini et al., American Journal of Clinical Pathology 117, 237-245, (2002); Thomaizy et al., J Mol Diagn 4, 201-208, (2002); Fontanilles et al. Acta Neuropathol Commun 8, 52, (2020); Xie et al., Oncol Lett 7, 131-136, (2014); Schrevel et al., Mod Pathol 24, 720-728, (2011).

TABLE III
Non-linear fit of a semi-log line from standard curves.

| | | Equation of semi-log | |
|---|---|---|---|
| | Plasmid construct | line | |
| Primer set | used as template | (x is log, y is linear) | R² |
| CDKN2A_129_NA | pCMV_p16_INK4A | Y = −1.698X + 43.6 | 0.865 |
| p53_123_NA | pDONR223_TP53_WT | Y = −1.587X + 40.76 | 0.880 |
| PIK3CA_119_NA | pDONR223_PIK3CA_WT | Y = −2.484X + 27.61 | 0.97 |
| HRAS_123_NA | pDONR223_HRAS_WT | Y = −1.737X + 21.53 | 0.968 |
| NRAS_125_NA | pDONR223_NRAS_WT | Y = −1.114X + 22.45 | 0.914 |
| FBXW7_129_NA | pDONR223_FBXW7 | Y = −2.081X + 24.10 | 0.984 |
| RP_CDC | RNase P | Y = −3.116X + 34.14 | 0.97 |

TABLE IV
Validated primers for HPV-mediated HNSCC.

| | | |
|---|---|---|
| HPV16_E7_RCC_Fwd | 5′-GATGAAATAGATGGTCCAGC-3′ | SEQ ID NO: 54 |
| HPV16_E7_RCC_Rev | 5′-GCTTTGTACGCACAACCGAAGC-3′ | SEQ ID NO: 55 |
| HPV16_E6_NNK_Fwd | 5′-GCAATGTTTCAGGACCCACA-3′ | SEQ ID NO: 56 |
| HPV16_E6_NNK_Rev | 5′-CGCAGTAACTGTTGCTTGCAGT-3′ | SEQ ID NO: 57 |
| HPV16-E7-NA-Fwd | 5′-TGTGACTCTACGCTTCGGTTG-3′ | SEQ ID NO: 58 |
| HPV16-E7-NA-Rev | 5′-GCCCATTAACAGGTCTTCCA-3′ | SEQ ID NO: 59 |
| HPV18_E7_NA_Fwd | 5′-GCATGGACCTAAGGCAACAT-3′ | SEQ ID NO: 60 |
| HPV18_E7_NA_Rev | 5′-GAAGGTCAACCGGAATTTCAT-3′ | SEQ ID NO: 61 |
| 18S_DR_Fwd | 5′-GTAACCCGTTGAACCCCATT-3′ | SEQ ID NO: 62 |
| 18S_DR_Rev | 5′-CCATCCAATCGGTAGTAGCG-3′ | SEQ ID NO: 63 |
| b-Actin_DR_Fwd | 5′-TGACATTAAGGAGAAGCTGTCCTAC-3′ | SEQ ID NO: 64 |
| b-Actin_DR_Rev | 5′-GAGTTGAAGGTAGTTTCGTGGATG-3′ | SEQ ID NO: 65 |

TABLE V
Primers and amplification regions.

| Target | | | Binding | Reference |
|---|---|---|---|---|
| name | Primer name | primer sequence (5′-3′) | site | sequence |
| pHPV16 | HPV16_E7_ | 5′-GATGAAATAGATGGTCCAGC- | 667-774 | HPV16_complete_seq_ |
| | RCC_F | 3′(SEQ ID NO: 66) | | LC718903.1; |
| | HPV16_E7_ | 5′-GCTTTGTACGCACAACCGAAGC- | | hpv16_E7_complete_cds |
| | RCC_R | 3′(SEQ ID NO: 67) | | |
| | HPV16_E6_ | 5′-GCAATGTTTCAGGACCCACA- | 101-219 | HPV16_complete_seq_ |
| | NNK_F | 3′(SEQ ID NO: 68) | | U89348.1; hpv16_E6_ |
| | HPV16_E6_ | 5′-CGCAGTAACTGTTGCTTGCAGT- | | complete_cds |
| | NNK_R | 3′(SEQ ID NO: 69) | | |
| | HPV16_E7_ | 5′-TGTGACTCTACGCTTCGGTTG- | 742-816 | HPV16_complete_seq_ |
| | NA_F | 3′(SEQ ID NO: 70) | | U89348.1; hpv16_E7_ |
| | HPV16_E7_ | 5′-GCCCATTAACAGGTCTTCCA-3′ | | complete_cds |
| | NA_R | (SEQ ID NO: 71) | | |
| pHPV18 | HPV18_E7_ | 5′-GCATGGACCTAAGGCAACAT- | 592-665 | HPV18 K1872_complete_ |
| | NA_F | 3′(SEQ ID NO: 72) | | genome |
| | HPV18_E7 | 5′-GAAGGTCAACCGGAATTTCAT-3′ | | |
| | NA R | (SEQ ID NO: 73) | | |
| CDKN2A | CDKN2A_129_ | 5′- | 188263- | Homo sapiens |
| | NA_F | ACAAATTCTCAGATCATCAGTCCTC- | 188391 | gene for |
| | | 3′(SEQ ID NO: 74) | | p16/CDKN2A, |
| | CDKN2A_129_ | 5′-AGGAGCTGGGCCATCG-3′ | | complete cds |
| | NA_R | (SEQ ID NO: 75) | | AB060808.1 |
| TP53 | p53_123_ | 5′-TCATCTTGGGCCTGTGTTATC- | 2490- | Homo_sapiens_p53_ |
| | NA_F | 3′(SEQ ID NO: 76) | 2612 | mRNA_complete_ |
| | p53_123_ | 5′-TGTGATGATGGTGAGGATGG- | | CDS |
| | NA_R | 3′(SEQ ID NO: 77) | | AH007665.2 |
| TP53 | p53_127_ | 5′-TGGCTCTGACTGTACCACCATC- | 2519- | Homo_sapiens_p53_ |
| | NA_F | 3′(SEQ ID NO: 78 ) | 2645 | mRNA_complete_CDS_ |
| | p53_127_ | 5′-GTGGCAAGTGGCTCCTGA-3′ | | AH007665.2 |
| | NA_R | (SEQ ID NO: 79) | | |
| PIK3CA | PIK3CA 119_ | 5′-CCCCCTCCATCAACTTCTTC- | 491-609 | Homo_sapiens_PIK3CA_ |
| | NA_F | 3′(SEQ ID NO: 80) | | mRNA_complete_CDS_ |
| | PIK3CA 119_ | 5′-GAAAAAGCCGAAGGTCACAA- | | NM_006218.4 |
| | NA_R | 3′(SEQ ID NO: 81) | | |
| | PIK3CA_129_ | 5′-TTTGATGACATTGCATACATTCG- | 3369- | Homo_sapiens_PIK3CA_ |
| | NA_F | 3′ (SEQ ID NO: 82) | 3497 | mRNA_complete_CDS_ |
| | | | | NM_006218.4 |
| | PIK3CA_129_ | 5′- | | |
| | NA_R | GATCCAATCCATTTTTGTTGTCCAG- | | |
| | | 3′(SEQ ID NO: 83) | | |
| HRAS | HRAS_123_ | 5′-GTGGTCATTGATGGGGAGAC- | 38-160 | Homo_sapiens_HRAS_ |
| | NA_F | 3′(SEQ ID NO: 84) | | complete_CDS_ |
| | HRAS_123_ | 5′-GATGGCAAACACACACAGGA- | | MZ068328.1 |
| | NA_R | 3′(SEQ ID NO: 85) | | |
| | HRAS_2HK_F | 5′-GACGGAATATAAGCTGGTGG- | 1204- | Homo_sapiens_HRAS_ |
| | | 3′(SEQ ID NO: 86) | 3281 | complete_CDS_ |
| | HRAS_2HK_R | 5′-AGGCACGTCTCCCCATCAAT- | | NM_005343.4 |
| | | 3′(SEQ ID NO: 87) | | |
| NRAS | NRAS_125_ | 5′-ACACCCCCAGGATTCTTACAG-3′ | 4864- | Homo_sapiens_NRAS_ |
| | NA_F | (SEQ ID NO: 88) | 4988 | EU332857_complete_ |
| | NRAS_125 | 5′-CGCCTGTCCTCATGTATTGG- | | CDS |
| | NA_R | 3′(SEQ ID NO: 89) | | |
| | NRAS_122 | 5′-GATGTGGCTCGCCAATTAAC-3′ | 2633- | Homo_sapiens_ |
| | NA_F | (SEQ ID NO: 90) | 2754 | NRAS_EU332857_ |
| | NRAS_122 | 5′-GATTGTCAGTGCGCTTTTCC- | | complete_CDS |
| | NA_R | 3′(SEQ ID NO: 91) | | |
| KRAS | KRAS_MH_F | 5′- | 1-567 | Homo_sapiens_KRAS_ |
| | | ATGACTGAATATAAACTTGTGGTA | | mRNA_AF493917_ |
| | | G-3′ (SEQ ID NO: 92) | | complete_CDS |
| | KRAS_MH_R | 5′- | | |
| | | TTACATAATTACACACTTTGTCTTT | | |
| | | GAC-3′ (SEQ ID NO: 93) | | |
| FBXW7 | FBXW7_129_ | 5′TTGAGACAGGCCAGTGTTTACAT- | 1625- | Homo_sapiens_FBXW7_ |
| | NA_F | 3′(SEQ ID NO: 94) | 1753 | variant1_mRNA_ |
| | FBXW7_129_ | 5′-CAGTCTCTGGATCCCACACC- | | NM_033632.3 |
| | NA_R | 3′(SEQ ID NO: 95) | | |
| CCND1 | CCND1_PEP_F | 5′-GCAGAAGGAGGTCCTGCCGT- | 280-856 | Homo_sapiens_ |
| | | 3′(SEQ ID NO: 96) | | cyclinD1_mRNA_ |
| | CCND1_PEP_R | 5′-GTCACACTTGATCACTCTGG- | | complete_CDS_BC023620.2 |
| | | 3′(SEQ ID NO: 97) | | |
| | CCND1_VAT_F | 5′-CCGTCCATGCGGAAGATC-3′ | 296-370 | Homo_sapiens_ |
| | | (SEQ ID NO: 98) | | cyclinD1_mRNA_ |
| | CCND1_VAT_R | 5′-GAAGACCTCCTCCTCGCACT- | | complete_ |
| | | 3′(SEQ ID NO: 99) | | CDS_BC023620.2 |
| EGFR | EGFR_2_MF_F | 5′-ATGCAAATAAAACCGGACTGA- | 638-846 | Homo_sapiens_ |
| | | 3′(SEQ ID NO: 100) | | EGFR_variant1_mRNA_ |
| | EGFR_MS_R | 5′-GGGACAGCTTGGATCACACT- | | NM_005228.5 |
| | | 3′(SEQ ID NO: 101) | | |
| | EGFR_JX_F | 5′-TGACTGAGGACAGCATAGACGA- | 3491-3693 | Homo_sapiens_ |
| | | 3′(SEQ ID NO: 102) | | EGFR_variant1_mRNA_ |
| | EGFR_JX_R | 5′-GGGCTGGACAGTGTTGAGATAC- | | NM_005228.5 |
| | | 3′(SEQ ID NO: 103) | | |
| | EGFR_MS_F | 5′-CATGTCGATGGACTTCCAGA- | 785-846 | Homo_sapiens_ |
| | | 3′(SEQ ID NO: 104) | | EGFR_variant1_mRNA_ |
| | EGFR_MS_R | 5′-GGGACAGCTTGGATCACACT- | | NM_005228.5 |
| | | 3′(SEQ ID NO: 105) | | |

TABLE VI
Target sequences and reference Accession numbers

| | | Accession | | | Accession |
|---|---|---|---|---|---|
| | | Number | | | Number |
| | | (NCBI | | | (NCBI |
| Target | | Nucleotide | Target | | Nucleotide |
| name | Primer name | Database) | name | Primer name | Database) |
| pHPV16 | HPV16_E7_RCC_F | MW388716.1 | NRAS | NRAS_125_NA_F | EU332857.1 |
| pHPV18 | HPV16_E7_RCC_R | | | NRAS_125_NA_R | |
| | HPV16_E6_NNK_F | MZ447801.1 | | NRAS_122_NA_F | EU332857.1 |
| | HPV16_E6_NNK_R | | | NRAS_122_NA_R | |
| | HPV18_E7_NA_F | LC509006.1 | KRAS | KRAS_MH_F | AF493917.1 |
| | HPV18_E7_NA_R | | | KRAS_MH_R | |
| CDKN2A | CDKN2A_129_NA_F | AB060808.1 | FBXW7 | FBXW7_129_NA_F | NM_033632.3 |
| TP53 | CDKN2A_129_NA_R | | | FBXW7_129_NA_R | |
| | p53_127_NA_F | AH007665.2 | CCND1 | CCND1_PEP_F | BC023620.2 |
| | p53_127_NA_R | | | CCND1_PEP_R | |
| PIK3CA | PIK3CA_119_NA_F | NM_006218.4 | | CCND1_VAT_F | BC023620.2 |
| | PIK3CA_119_NA_R | | | CCND1_VAT_R | |
| | PIK3CA_129_NA_F | NM_006218.4 | EGFR | EGFR_2_MF_F | NM_005228.5 |
| | PIK3CA_129_NA_R | | | EGFR_MS_R | |
| HRAS | HRAS_123_NA_F | MZ068328.1 | | EGFR_JX_F | NM_005228.5 |
| | HRAS_123_NA_R | | | EGFR_JX_R | |
| | HRAS_2HK_F | NM_005343.4 | | EGFR_MS_F | NM_005228.5 |
| | HRAS_2HK_R | | | EGFR_MS_R | |

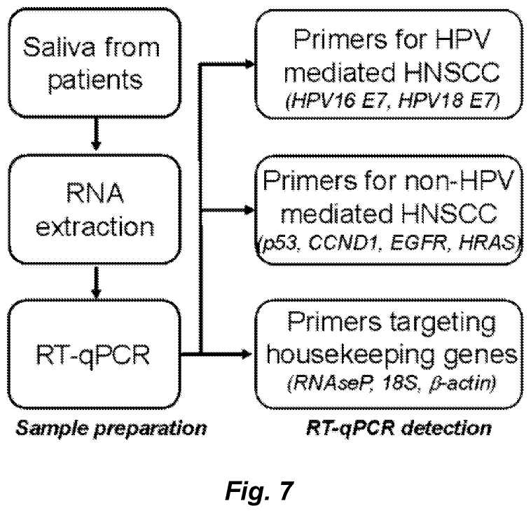

FIG. 7 summarizes our workflow for detection of 1) genes involved in HPV-mediated HNSCC, 2) non-HPV-mediated HNSCC, and 3) housekeeping genes from a single saliva sample, with a potential for combining promising targets into a multiplex PCR system. As demonstrated above, the analysis and interpretation of results will depend on whether the gene target is up-regulated (calculate fold changes using 2^{-ΔΔCt} method) or exhibited INDEL mutations (direct reporting of presence/absence of gene target, i.e., raw Ct values) in HNSCC patient samples.

Polynucleotide sequences.
HPV16 genome (U89348.1)
(SEQ ID NO: 1)
actacaataatccatgtataaaactaagggcgtaaccgaaatcggttgaaccgaaaccggttagtataaaagcagacattttatgcacca
aaagagaactgcaatgtttcaggacccacaggagcgacccggaaagttaccacagttatgcacagagctgcaaacaactatacatga
tataatattagaatgtgtgtactgcaagcaacagttactgcgacgtgaggtatatgactttgcttttcgggatttatgcatagtatatagagat
gggaatccatatgctgtatgtgataaatgtttaaagttttattctaaaattagtgagtatagacattattgttatagtgtgtatggaacaacatta
gaacagcaatacaacaaaccgttgtgtgatttgttaattaggtgtattaactgtcaaaagccactgtgtcctgaagaaaagcaaagacatc
tggacaaaaagcaaagattccataatataaggggtcggtggaccggtcgatgtatgtcttgttgcagatcatcaagaacacgtagagaa
acccagctgtaatcatgcatggagatacacctacattgcatgaatatatgttagatttgcaaccagagacaactgatctctactgttatgag
caattaaatgacagctcagaggaggaggatgaaatagatggtccagctggacaagcagaaccggacagagcccattacaatattgta
accttttgttgcaagtgtgactctacgcttcggttgtgcgtacaaagcacacacgtagacattcgtactttggaagacctgttaatgggcac
actaggaattgtgtgccccatctgttctcagaaaccataatctaccatggctgatcctgcaggtaccaatggggaagagggtacgggat
gtaatggatggttttatgtagaggctgtagtggaaaaaaaaacaggggatgctatatcagatgacgagaacgaaaatgacagtgatac
aggtgaagatttggtagattttatagtaaatgataatgattatttaacacaggcagaaacagagacagcacatgcgttgtttactgcacag
gaagcaaaacaacatagagatgcagtacaggttctaaaacgaaagtatttgggtagtccacttagtgatattagtggatgtgtagacaat
aatattagtcctagattaaaagctatatgtatagaaaaacaaagtagagctgcaaaaaggagattatttgaaagcaaagacagcgggtat
ggcaatactgaagtggaaactcagcagatgttacaggtagaagggcgccatgagactgaaacaccatgtagtcagtatagtggtgga
agtgggggtggttgcagtcagtacagtagtggaagtgggggagagggtgttagtgaaagacacaatatatgccaaacaccacttaca
aatattttaaatgtactaaaaactagtaatgcaaaggcagcaatgttagcaaaatttaaagagttatacggggtgagttttacagaattagt
aagaccatttaaaagtaataaatcaacgtgttgcgattggtgtattgctgcatttggacttacacccagtatagctgacagtataaaaacac
tattacaacaatattgtttatatttacacattcaaagtttagcatgttcatggggaatggttgtgttactattagtaagatataaatgtggaaaaa
atagagaaacaattgaaaaattgctgtctaaactattatgtgtgtctccaatgtgtatgatgatagagcctccaaaattgcgtagtacagca
gcagcattatattggtataaaacaggtatatcaaatattagtgaagtgtatggagacacgccagaatggatacaaagacaaacagtatta
caacatagttttaatgattgtacatttgaattatcacagatggtacaatgggcctacgataatgacatagtagacgatagtgaaattgcatat
aaatatgcacaattggcagacactaatagtaatgcaagtgcctttctaaaaagtaattcacaggcaaaaattgtaaaggattgtgcaaca
atgtgtagacattataaacgagcagaaaaaaaacaaatgagtatgagtcaatggataaaatatagatgtgatagggtagatgatggagg
tgattggaagcaaattgttatgtttttaaggtatcaaggtgtagagtttatgtcatttttaactgcattaaaaagatttttgcaaggcatacctaa
aaaaaattgcatattactatatggtgcagctaacacaggtaaatcattatttggtatgagtttaatgaaatttctgcaagggtctgtaatatgtt
ttgtaaattctaaaagccatttttggttacaaccattagcagatgccaaaataggtatgttagatgatgctacagtgccctgttggaactatat
agatgacaatttaagaaatgcattggatggaaatttagtttctatggatgtaaagcatagaccattggtacaactaaaatgccctccattatt
aattacatctaacattaatgctggtacagattctaggtggccttatttacataatagattggtggtgtttacatttcctaatgagtttccatttga
cgaaaacggaaatccagtgtatgagcttaatgataagaactggaaatcctttttctcaaggacgtggtccagattaagtttgcacgagga
cgaggacaaggaaaacgatggagactctttgccaacgtttaaatgtgtgtcaggacaaaatactaacacattatgaaaatgatagtaca
gacctacgtgaccatatagactattggaaacacatgcgcctagaatgtgctatttattacaaggccagagaaatgggatttaaacatatta
accaccaggtggtgccaacgctggctgtatcaaagaataaagcattacaagcaattgaactgcaactaacgttagaaacaatatataac
tcacaatatagtaatgaaaagtggacattacaagacgttagccttgaagtgtatttaactgcaccaacaggatgtataaaaaaacatggat
atacagtggaagtgcagtttgatggagacatatgcaatacaatgcattatacaaactggacacatatatatatttgtgaagaagcatcagt
aactgtggtagagggtcaagttgactattatggtttatattatgttcatgaaggaatacgaacatattttgtgcagtttaaagatgatgcaga
aaaatatagtaaaaataaagtatgggaagttcatgcgggtggtcaggtaatattatgtcctacatctgtgtttagcagcaacgaagtatcct
ctcctgaaactattaggcagcacttggccaaccactccgccgcgacccataccaaagccgtcgccttgggcaccgaagaaacacag
acgactatccagcgaccaagatcagagccagacaccggaaacccctgccacaccactaagttgttgcacagagactcagtggacag
tgctccaatcctcactgcatttaacagctcacacaaaggacggattaactgtaatagtaacactacacccatagtacatttaaaaggtgat
gctaatactttaaaatgtttaagatatagatttaaaaagcattgtaaattgtatactgcagtgtcgtctacatggcattggacaggacataatg
taaaacataaaagtgcaattgttacacttacatatgatagtgaatggcaacgtgaccaatttttgtctcaagttaaaataccaaaaactatta
cagtgtctactggatttatgtctatatgacaaatcttgatactgcatacacaacattactggcgtgctttttgctttgcttttgtgtgcttttgtgt
gtctgcctattaatacgtccgctgcttttgtctgtgtctacatacacatcattaatactattggtattactattgtggataacagcagcctctgcgt
ttaggtgttttattgtatatattgtatttgtttatataccattatttttaatacatacacatgcacgctttttaattacataatgtatatgtacataat
gtaattgttacatataattgttgtataccataacttactattttttcttttttatttttatatataatttttttttggtttgtttgtttgtttttt
aataaactgttctcacttaacaatgcgacacaaacgttctgcaaaacgcacaaaacgtgcatcggctacccaactttataaaacatgcaaacaggcagg
tacatgtccacctgacattatacctaaggttgaaggcaaaactattgctgatcaaatattacaatatggaagtatgggtgtattttttggtgg
gttaggaattggaacagggtcgggtacaggcggacgcactgggtatattccattgggaacaaggcctcccacagctacagatacactt
gctcctgtaagaccccctttaacagtagatcctgtgggcccttctgatccttctatagtttctttagtggaagaaactagttttattgatgctg
gtgcaccaacatctgtaccttccatccccccagatgtatcaggatttagtattactacttcaactgataccacacctgctatattagatattaa
taatactgttactactgttactacacataataatcccactttcactgacccatctgtattgcagcctccaacacctgcagaaactggagggc
attttacactttcatcatccactattagtacacataattatgaagaaattcctatggatacatttattgttagcacaaaccctaacacagtaact
agtagcacacccataccagggtctcgcccagtggcacgcctaggattatatagtcgcacaacacaacaagttaaagttgtagaccctg
cttttgtaaccactcccactaaacttattacatatgataatcctgcatatgaaggtatagatgtggataatacattatattttcctagtaatgata
atagtattaatatagctccagatcctgactttttggatatagttgctttacataggccagcattaacctctaggcgtactggcattaggtaca
gtagaattggtaataaacaaacactacgtactcgtagtggaaaatctataggtgctaaggtacattattattatgatttgagtactattgatcc
tgcagaagaaatagaattacaaactataacaccttctacatatactaccacttcacatgcagcctcacctacttctattaataatggcttatat
gatatttatgcagatgactttattacagatacttctacaaccccggtaccatctgtaccctctacatctttatcaggttatattcctgcaaatac
aacaattccttttggtggtgcatacaatattcctttagtatcaggtcctgatatacccattaatataactgaccaagctccttcattaattcctat
agttccagggtctccacaatatacaattattgctgatgcaggtgacttttatttacatcctagttattacatgttacgaaaacgacgtaaacgt
ttaccatattttttttcagatgtctctttggctgcctagtgaggccactgtctacttgcctcctgtcccagtatctaaggttgtaagcacggatg
aatatgttgcacgcacaaacatatattatcatgcaggaacatccagactacttgcagttggacatccctattttcctattaaaaaacctaaca
ataacaaaatattagttcctaaagtatcaggattacaatacagggtatttagaatacatttacctgaccccaataagtttggttttcctgacac
ctcattttataatccagatacacagcggctggtttgggcctgtgtaggtgttgaggtaggtcgtggtcagccattaggtgtgggcattagt
ggccatcctttattaaataaattggatgacacagaaaatgctagtgcttatgcagcaaatgcaggtgtggataatagagaatgtatatctat
ggattacaaacaaacacaattgtgtttaattggttgcaaaccacctataggggaacactggggcaaaggatccccatgtaccaatgttgc
agtaaatccaggtgattgtccaccattagagttaataaacacagttattcaggatggtgatatggttgatactggctttggtgctatggactt
tactacattacaggctaacaaaagtgaagttccactggatatttgtacatctatttgcaaatatccagattatattaaaatggtgtcagaacc
atatggcgacagcttatttttttatttacgaagggaacaaatgtttgttagacatttatttaatagggctggtgctgttggtgaaaatgtacca
gacgatttatacattaaaggctctgggtctactgcaaatttagccagttcaaattattttcctacacctagtggttctatggttacctctgatgc
ccaaatattcaataaaccttattggttacaacgagcacagggccacaataatggcatttgttggggtaaccaactatttgttactgttgttga
tactacacgcagtacaaatatgtcattatgtgctgccatatctacttcagaaactacatataaaaatactaactttaaggagtacctacgaca
tggggaggaatatgatttacagtttatttttcaactgtgcaaaataaccttaactgcagacgttatgacatacatacattctatgaattccact
attttggaggactggaattttggtctacaaccccccccaggaggcacactagaagatacttataggtttgtaacatcccaggcaattgctt
gtcaaaaacatacacctccagcacctaaagaagatccccttaaaaaatacactttttgggaagtaaatttaaaggaaaagttttctgcaga
cctagatcagtttcctttaggacgcaaatttttactacaagcaggattgaaggccaaaccaaaatttacattaggaaaacgaaaagctaca
cccaccacctcatctacctctacaactgctaaacgcaaaaaacgtaagctgtaagtattgtatgtatgttgaattagtgttgtttgttgtttata
tgtttgtatgtgcttgtatgtgcttgtaaatattaagttgtatgtgtgtttgtatgtatggtataataaacacgtgtgtatgtgtttttaaatgcttgt
gtaactattgtgtgatgcaacataaataaacttattgtttcaacacctactaattgtgttgtggttattcattgtatataaactatatttgctacaat
ctgtttttgttttatatatactatattttgtagcgccagcggccattttgtagcttcaaccgaattcggttgcatgctttttggcacaaaatgtgttt
ttttaaatagttctatgtcagcaactatagtttaaacttgtacgtttcctgcttgccatgcgtgccaaatccctgttttcctgacctgcactgctt
gccaaccattccattgttttttacactgcactatgtgcaactactgaatcactatgtacattgtgtcatataaaataaatcactatgegccaac
gccttacataccgctgttaggcacatatttttggcttgttttaactaacctaattgcatatttggcataaggtttaaacttctaaggccaactaa
atgtcaccctagttcatacatgaactgtgtaaaggttagtcatacattgttcatttgtaaaactgcacatgggtgtgtgcaaaccgttttgggt
tacacatttacaagcaacttatataataatactaa
HPV18 genome (LC509006.1)
(SEQ ID NO: 2)
attaatacttttaacaattgtagtatataaaaaagggagtaaccgaaaacggtcgggaccgaaaacggtgtatataaaagatgtgagaaa
cacaccacaatactatggcgcgctttgaggatccaacacggcgaccctacaagctacctgatctgtgcacggaactgaacacttcact
gcaagacatagaaataacctgtgtatattgcaagacagtattggaacttacagaggtatttgaatttgcatttaaagatttatttgtggtgtat
agagacagtataccgcatgctgcatgccataaatgtatagatttttattctagaattagagaattaagacattattcagactctgtgtatgga
gacacattggaaaaactaactaacactgggttatacaatttattaataaggtgcctgcggtgccagaaaccgttgaatccagcagaaaa
acttagacaccttaatgaaaaacgacgatttcacaacatagctgggcactatagaggccagtgccattcgtgctgcaaccgagcacga
caggaacgactccaacgacgcagagaaacacaagtataatattaagtatgcatggacctaaggcaacattgcaagacattgtattgcat
ttagagccccaaaatgaaattccggttgaccttctatgtcacgagcaattaagcgactcagaggaagaaaacgatgaaatagatggagt
taatcatcaacatttaccagcccgacgagccgaaccacaacgtcacacaatgttgtgtatgtgttgtaagtgtgaagccagaattgagct
agtagtagaaagctcagcagacgaccttcgagcattccagcagctgtttctgaacaccctgtcctttgtgtgtccgtggtgtgcatccca
gcagtaagcaacaatggctgatccagaaggtacagacggggagggcacgggttgtaacggctggttttatgtacaagctattgtagac
aaaaaaacaggagatgtaatatcagatgacgaggacgaaaatgcaacagacacagggtcggatatggtagattttattgatacacaag
gaacattttgtgaacaggcagagctagagacagcacaggcattgttccatgcgcaggaggtccacaatgatgcacaagtgttgcatgtt
ttaaaacgaaagtttgcaggaggcagcaaagaaaacagtccattaggggagcggctggaggtggatacagagttaagtccacggtta
caagaaatatcgttaaatagtgggcagaaaaaggcaaaaaggcggctgtttacaatatcagatagtggctatggctgttctgaagtgga
agcaacacagattcaggtaactacaaatggcgaacatggcggcaatgtatgtagtggcggcagtacggaggctatagacaacgggg
gcacagagggcaacaccagcagtgtagacggtacacgtgacaatagcaatatagaaaatgtaaatccacaatgtaccatagcacaatt
aaaagacttgttaaaagtaaacaataaacaaggagctatgttagcagtatttaaagacacatatgggctatcatttacagatttagttagaa
attttaaaagtgataaaaccacgtgtacagattgggttacagctatatttggagtaaacccaacaatagcagaaggatttaaaacactaat
acagccatttatattatatgcccatattcaatgtctagactgtaaatggggagtattaatattagccctgttgcgttacaaatgtggtaagagt
agactaacagttgctaaaggtttaagtacgttgttacacgtacctgaaacttgtatgttaattcaaccaccaaaattgcgaagtagtgttgc
agcactatattggtatagaacaggaatatcaaatattagtgaagtaatgggagacacacctgagtggatacaaagacttactattatacaa
catggaatagatgatagcaattttgatttgtcagaaatggtacaatgggcatttgataatgagctgacagatgaaagcgatatggcatttg
aatatgccttattagcagacagcaacagcaatgcagctgcctttttaaaaagcaattgccaagctaaatatttaaaagattgtgccacaat
gtgcaaacattataggcgagcccaaaaacgacaaatgaatatgtcacagtggatacgatttagatgttcaaaaatagatgaaggggga
gattggagaccaatagtgcaattcctgcgataccaacaaatagagtttataacatttttaggagccttaaaattatttttaaaaggaacccc
caaaaaaaattgtttagtattttgtggaccagcaaatacaggaaaatcatattttggaatgagttttatacactttatacaaggagcagtaat
atcatttgtgaattccactagtcatttttggttggaaccgttaacagatactaaggtggccatgttagatgatgcaacgaccacgtgttgga
catactttgatacctatatgagaaatgcgttagatggcaatccaataagtattgatagaaagcacaaaccattaatacaactaaaatgtcct
ccaatactactaaccacaaatatacatccagcaaaggataatagatggccatatttagaaagtagaataacagtatttgaatttccaaatg
catttccatttgataaaaatggcaatccagtatatgaaataaatgacaaaaattggaaatgtttttttgaaaggacatggtccagattagattt
gcacgaggaagaggaagatgcagacaccgaaggaaaccctttcggaacgtttaagtgcgttgcaggacaaaatcatagaccactat
gaaaatgacagtaaagacatagacagccaaatacagtattggcaactaatacgttgggaaaatgcaatattctttgcagcaagggaaca
tggcatacagacattaaaccaccaggtggtgccagcctataacatttcaaaaagtaaagcacataaagctattgaactgcaaatggccc
tacaaggccttgcacaaagtgcatacaaaaccgaggattggacactgcaagacacatgcgaggaactatggaatacagaacctactc
actgctttaaaaaaggtggccaaacagtacaagtatattttgatggcaacaaagacaattgtatgaactatgtagcatgggacagtgtgta
ttatatgactgatgcaggaacatgggacaaaacggctacctgtgtaagtcacaggggattgtattatgtaaaggaagggtacaacacgt
tttatatagaatttaaaagtgaatgtgaaaaatatgggaacacaggtacgtgggaagtacattttgggaataatgtaattgattgtaatgact
ctatgtgcagtaccagtgacgacacggtatccgctactcagcttgttaaacagctacagcacaccccctcaccgtattccagcaccgtgt
ccgtgggcaccgcaaagacctacggccagacgtcggctgctacacgacctggacactgtggactcgcggagaagcagcattgtgg
acctgtcaacccacttctcggtgcagctacacctacaggcaacaacaaaagacggaaactctgtagtggtaacactacgcctataatac
atttaaaaggtgacagaaacagtttaaaatgtttacggtacagattgcgaaaacatagcgaccactatagagatatatcatccacctggc
attggacaggtgcaggcaatgaaaaaacaggaatactgactgtaacataccatagtgaaacacaaagaacaaaatttttaaatactgttg
caattccagatagtgtacaaatattggtgggatacatgacaatgtaatacatatgctgtagtaccaatatgttatcacttatttttttattttgctt
ttgtgtatgcatgtatgtgtgctgccatgtcccgcttttgccatctgtctgtatgtgtgcgtatgcatgggtattggtatttgtgtatattgtggt
aataacgtcccctgccacagcattcacagtatatgtattttgttttttattgcccatgttactattgcatatacatgctatattgtctttacagtaat
tgtataggttgttttatacagtgtattgtacattgtatattttgttttataccttttatgctttttgtatttttgtaataaaagtatggtatcccaccgtg
ccgcacgacgcaaacgggcttcggtaactgacttatataaaacatgtaaacaatctggtacatgtccacctgatgttgttcctaaggtgg
agggcaccacgttagcagataaaatattgcaatggtcaagccttggtatatttttgggtggacttggcataggtactggcagtggtacag
ggggtcgtacagggtacattccattggggggcgttccaatacagtggtggatgttggtcctacacgtcccccagtggttattgaacctg
tgggccccacagacccatctattgttacattaatagaggactccagtgtggttacatcaggtgcacctaggcctacgtttactggcacgtc
tgggtttgatataacatctgcgggtacaactacacctgcggttttggatatcacaccttcgtctacctctgtgtctatttccacaaccaatttta
ccaatcctgcattttctgatccgtccattattgaagttccacaaactggggaggtgtcaggtaatgtatttgttggtacccctacatctggaa
cacatgggtatgaggaaatacctttacaaacatttgcttcttctggtacaggggaggaacccattagtagtaccccattgcctactgtgcg
gcgtgtagcaggtccccgcctttacagtagggcctaccaacaagtgtcagtggctaaccctgagtttcttacacgtccatcctctttaatta
catatgacaacccggcctttgagcctgtggacactacattaacatttgatcctcgtagtgatgttcctgattcagattttatggatattatccg
tctacataggcctgctttaacatccaggcgtgggactgttcgctttagtagattaggtcaaagggcaactatgtttacccgcagcggtaca
caaataggtgctagggttcacttttatcatgatataagtcctattgcaccttccccagaatatattgaactgcagcctttagtatctgccacg
gaggacaatgacttgtttgatatatatgcagatgacatggaccctgcagtgcctgtaccatcgcgttctactacctcctttgcattttctaaat
attcgcccactatatcttctgcctcttcctatagtaatgtaacggtccctttaacctcctcttgggatgtgcctgtatacacgggtcctgatatt
acattaccatctactacctctgtatggcccattgtatcacccacagcccctgcctctacacagtatattggtatacatggtacacattattatt
tgtggccattatattattttattcctaagaaacgtaaacgtgttccctatttttttgcagatggctttgtggcggcctagtgacaataccgtatat
cttccacctccttctgtggcaagagttgtaaataccgatgattatgtgactcgcacaagcatattttatcatgctggcagctctagattattaa
ctgttggtaatccatattttagggttcctgcaggtggtggcaataagcaggatattcctaaggtttctgcataccaatatagagtatttaggg
tgcagttacctgacccaaataaatttggtttacctgatactagtatttataatcctgaaacacaacgtttagtgtgggcctgtgctggagtgg
aaattggccgtggtcagcctttaggtgttggccttagtgggcatccattttataataaattagatgacactgaaagttcccatgccgccacg
tctaatgtttctgaggacgttagggacaatgtgtctgtagattataagcagacacagttatgtattttgggctgtgcccctgctattggggaa
cactgggctaaaggcactgcttgtaaatcgcgtcctttatcacagggcgattgcccccctttagaacttaaaaacacagttttggaagatg
gtgatatggtagatactggatatggtgccatggactttagtacattgcaagatactaaatgtgaggtaccattggatatttgtcagtctatttg
taaatatcctgattatttacaaatgtctgcagatccttatggggattccatgtttttttgcttacggcgtgagcagctttttgctaggcatttttgg
aatagagcaggtactatgggtgacactgtgcctcaatccttatatattaaaggcacaggtatgcgtgcttcacctggcagctgtgtgtatt
ctccctctccaagtggctctattgttacctctgactcccagttgtttaataaaccatattggttacataaggcacagggtcataacaatggtg
tttgctggcataatcaattatttgttactgtggtagataccactcgcagtaccaatttaacaatatgtgcttctacacagtctcctgtacctgg
gcaatatgatgctaccaaatttaagcagtatagcagacatgttgaggaatatgatttgcagtttatttttcagttgtgtactattactttaactg
cagatgttatgtcctatattcatagtatgaatagcagtattttagaggattggaactttggtgttccccccccgccaactactagtttggtgga
tacatatcgttttgtacaatctgttgctattacctgtcaaaaggatgctgcaccggctgaaaataaggatccctatgataagttaaagttttg
gaatgtggatttaaaggaaaagttttctttagacttagatcaatatccccttggacgtaaatttttggttcaggctggattgcgtcgcaagcc
caccataggccctcgcaaacgttctgctccatctgccactacgtcttctaaacctgccaagcgtgtgcgtgtacgtgccaggaagtaata
tgtgtgtgtgtatatatatatacatctattgttgtgtttgtatgtcctgtgtttgtgtttgttgtatgattgcattgtatggtatgtatggttgttgttgt
atgttgtatgttactatatttgttggtatgtggcattaaataaaatatgttttgtggttctgtgtgttatgtggttgcgccctagtgagtaacaact
gtatttgtgtttgtggtatgggtgttgcttgttgggctatatattgtcctgtatttcaagttataaaactgcacaccttacagcatccattttatcc
tacaatcctccattttgctgtgcaaccgatttcggttgcctttggcttatgtctgtggttttctgcacaatacagtacgctggcactattgcaaa
ctttaatcttttgggcactgctcctacatattttgaacaattggcgcgcctctttggcgcatacaaggcgcacctggtattagtcattttcctgt
ccaggtgcgctacaacaattgcttgcataactatatccactccctaagtaataaaactgcttttaggcacatattttagtttgtttttacttaag
ctaattgcatacttggcttgtacaactactttcatgtccaacattctgtctacccttaacatgaactataatatgactaagctgtgcatacatag
tttatgcaaccgaaataggttgggcagcacatactatacttttc
HPV16 genome (LC718903.1)
(SEQ ID NO: 3)
actacaataattcatgtataaaattaagggcgtaaccgaaatcggttgaaccgaaaccggttagtataaaagcagacattttatgcacca
aaagagaactgcaatgtttcaggacccacaggagcgacccagaaagttaccacagttatgcacagagctgcaaacaactatacatga
gataatattagaatgtgtgtactgcaagcaacagttactgcgacgtgaggtatatgactttgcttttcgggatttatgcatagtatatagaga
tgggaatccatatgctgtatgtgataaatgtttaaagttttattctaaaattagtgagtatagacattattgttatagtttgtatggaacaacatta
gaacagcaatacaacaaaccgttgtgtgatttgttaattaggtgtattaactgtcaaaagccactgtgtcctgaagaaaagcaaagacatc
tggacaaaaagcaaagattccataatataaggggtcggtggaccggtcgatgtatgtcttgttgcagatcatcaagaacacgtagagaa
acccagctgtaatcatgcatggagatacacctacattgcatgaatatatgttagatttgcaaccagagacaactgatctctactgttatgag
caattaagtgacagctcagaggaggaggatgaaatagatggtccagctggacaagcagaaccggacagagcccattacaatattgta
accttttgttgcaagtgtgactctacgcttcggttgtgcgtacaaagcacacacgtagacattcgtactttggaagacctgttaatgggcac
actaggaattgtgtgccccatctgttcccagaaaccataatctaccatggctgatcctgcaggtaccaatggggaagagggtacgggat
gtaatggatggttttatgtagaggctgtagtggaaaaaaaaacaggggatgctatatcagatgacgagaacgaaaatgacagtgatac
aggtgaagatttggtagattttatagtacatgataatgattatttaacacaggcagaaacagagacagcacatgcgttgtttactgcacag
gaagcaaaacaacatagagatgcagtacaggttctaaaacgaaagtatttgggtagtccacttagtgatattagtggatgtgtagacaat
aatattagtcctagattaaaagctatatgtatagaaaaacaaagtagagctgcaaaaaggagattatttgaaagcgaagacagcgggtat
ggcaatactgaagtggaaactcagcagatgttacaggtagaagggcgccatgagactgaaacaccatgtagtcagtatagtggtgga
agtgggggtggttgcagtcagtacagtagtggaagtgggggagagggtgttagtgaaagacacactatatgccaaacaccacttaca
aatattttaaatgtactaaaaactagtaatgcaaaggcagcaatgttagcaaaatttaaagagttatacggggtaagtttttcagaattagta
agaccatttaaaagtaataaatcaacgtgttgcgattggtgtattgctgcatttggacttacacccagtatagctgacagtataaaaacact
attacaacaatattgtttatatttacacattcaaagtttagcatgttcatggggaatggttgtgttactattagtaagatataaatgtggaaaaa
atagagaaacaattgaaaaattgctgtctaaactattatgtgtgtctccaatgtgtatgatgatagagcctccaaaattgcgtagtacagca
gcagcattatattggtataaaacaggtatgtcaaatattagtgaagtgtatggagacacgccagaatggatacaaagacaaacagtatta
caacatagttttaatgattgtacatttgaattatcacagatggtacaatgggcctacgataatgacatagtagacgatagtgaaattgcatat
aaatatgcacaattggcagacactaatagtaatgcaagtgcctttctaaaaagtaattcacaggcaaaaattgtaaaggattgtgcaaca
atgtgtagacattataaacgagcagaaaaaaaacaaatgagtatgagtcaatggataaaatatagatgtgatagagtagatgatggagg
tgattggaagcaaattgttatgtttttaaggtatcaaggtgtagagtttatgtcatttttaactgcattaaaaagatttttgcaaggcatacctaa
aaaaaattgcatattactatatggtgcagctaacacaggtaaatcattatttggtatgagtttaataaaatttctgcaagggtctgtaatatgtt
ttgtaaattctaaaagccatttttggttacaaccattagcagatgccaaaataggtatgttagatgatgctacagtgccctgttggaactaca
tagatgacaatttaagaaatgcattggatggaaatttagtttctatggatgtaaagcatagaccattggtacaactaaaatgccctccattat
taattacatctaacattaatgctggtacagattctaggtggccttatttacataatagattggtggtgtttacatttcctaatgagtttccatttga
cgaaaacggaaatccagtgtatgagcttaatgataagaactggaaatcctttttctcaaggacgtggtccagattaagtttgcacgagga
cgaggacaaggaaaacgatggagactctttgccaacgtttaaatgtgtgtcaggacaaaatactaacacattatgaaaatgatagtaca
aacctacgtgaccatatagactattggaaacacatgcgcctagaatgtgctatttattacaaggccagagaaatgggatttaaacatatta
accaccaggtggtgccaacactggctgtatcaaagaataaagcattacaagcaattgaactgcaactaacgttagaaacaatatataac
tcacaatatagtaatgaaaagtggacattacaagacgttagccttgaagtgtatttaactgcaccaacaggatgtataaaaaaacatggat
atacagtggaagtgcagtttgatggagacatatgcaatacaatgcattatacaaactggaaacatatatatatttgtgaagaagcatcagt
aactgtggtagagggtcaagttgactattatggtttatattatgttcatgaaggaatacaaacatattttgtgcagtttaaagatgatgcaga
aaaatatagtaaaaataaagtatgggaagttcatgcgggtggtcaggtaatattatgtcctacatctgtgtttagcagcaacgaagtatcct
ctcctgaaactattaggcagcacttggccaaccactccgccgcgacccataccaaagccgtcgccttgggcaccaaagaaacacaga
cgactatccagcgaccaagatcagagccagacaccggaaacccctgccacaccactaagttgctgcacagagactcagtggacagt
gctttaatcctcactgcatttaacagctcacacaaaggacggattaactgtaatagtaacactacacccatagtacatttaaaaggtgatg
ctaatactttaaaatgtttaagatatagatttaaaaagcattgtaaattgtatactgcagtgtcgtctacatggcattggacaggacataatgt
aaaacataaaagtgcaattgttacacttacatatgatagtgaatggcaacgtgaacaatttttgtctcaagttaaaataccaaaaactattac
agtgtctactggatttatgtctatatgacaaatcttgatactgcatccacaacattactggcgtgctttttgctttgcttttgtgtgcttttgtgtgt
ctgcctattaatacgtccgctgcttttgtctgtgtctacatacacatcattaatagtattggtattactattgtggataacagcagcctctgcgtt
taggtgttttattgtatatattgtatttgtttatataccattatttttaatacatactcatgcacgctttttaattacataatgtatatgtacataatgta
attgttacatataattgttgtataccataacttactattttttttttttatttttatatataattttttgtttgtttgtgtgtttgttttttaataaactg
ttatcacttaacaatgcgacacaaacgttctgcaaaacgcacaaaacgtgcateggctacccaactttataaaacatgcaaacaggcaggtaca
tgtccacctgacattatacctaaggttgaaggcaaaactattgctgatcaaatattacaatatggaagtatgggtgtattttttggtgggtta
ggaattggaacagggtcgggtacaggcggacgcactgggtatattccattgggaacaaggcctcccacagctacagatacacttgct
cctgtaagaccccctttaacagtagatcctgtgggcccttctgatccttctatagtttctttagtggaagaaactagttttattgatgctggtg
caccaacatctgtaccttccattcccccagatgtatcaggatttagtattactacttcaactgataccacacctgctatattagatattaataat
actgttactactgttactacacataataatcccacttttactgacccatctgtattgcagcctccaacacctgcagaaactggagggcatttt
acactttcatcatccactattagtacacataattatgaagaaattcctatggatacatttattgttagcacaaaccctaacacagtaactagta
gcacacccataccagggtctcgcccagtggcacgcctaggattatatagtcgcacaacacaacaagttaaagttgtagaccctgctttt
gtaaccactcccactaaacttattacatatgataatcctgcatatgaaggtatagatgtggataatacattatattttcctaataatgataatag
tattaatatagctccagatcctgactttttggatatagttgctttacataggccagcattaacctctaggcgtactggcattaggtacagtag
aattggtaataaacaaacactacgtactcgtagtggaaaatctataggtgctaaggtacattattattatgatttcagtactattgatcctgca
gaagaaatagaattacaaactataacaccttctacatatactaccacttcacatgcagcctcacctacttctattaataatggattatatgata
tttatgcagatgactttattacagatacttttacaaccccggtaccatctgtaccctctacatctttatcaggttatattcctgcaaatacaaca
attccttttggtggtgcatacaatattcctttagtatcaggtcctgatatacccattaatataactgaccaagctccttcattacttcctatagttc
cagggtctccacaatatacaattattgctgatgcaggtgacttttatttacatcctagttattacatgttacgaaaacgacgtaaacgtttacc
atattttttttcagatgtctctttggctgcctagtgaggccactgtctacttgcctcctgtcccagtatctaaggttgtaagcacggatgaatat
gttgcacgcacaaacatatattatcatgcaggaacatccagactacttgcagttggacatccctattttcctattaaaaaacctaacaataa
caaaatattagttcctaaagtatcaggattacaatacagggtatttagaatacatttacctgaccccaataagtttggttttcctgacacctca
ttttataatccagatacacagcggctggtttgggcctgtgtaggtgttgaggtaggtcgtggtcagccattaggtgtgggcattagtggcc
atcctttattaaataaattggatgacacagaaaatgctagtgcttatgcagcaaatgcaggtgtggataatagagaatgtatatctatggatt
acaaacaaacacaattgtgtttaattggttgcaaaccacctataggggaacactggggcaaaggatccccatgtaccaatgttgcagta
aatccaggtgattgtccaccattagagttaataaacacagttattcaggatggtgatatggttgatactggctttggtgctatggactttact
acattacaggctaacaaaagtgaagttccactggatatttgtacatctatttgcaaatatccagattatattaaaatggtgtcagaaccatat
ggcgacagcttatttttttatttacgaagggaacaaatgtttgttagacatttatttaatagggctggtgctgttggtgaaaatgtaccagacg
atttatacattaaaggctctgggtctactgcaaatttagccagttcaaattattttcctacacctagtggttctatggttacctctgatgcccaa
atattcaataaaccttattggttacaacgagcacagggccacaataatggcatttgttggggtaaccaactatttgttactgttgttgatacta
cacgcagtacaaatatgtcattatgtgctgccatatctacttcagaaactacatataaaaatactaactttaaggagtacctacgacatggg
gaggaatatgatttacagtttatttttcaactgtgcaaaataaccttaactgcagacgttatgacatacatacattctatgaattccactattttg
gaggactggaattttggtctacaacctcccccaggaggcacactagaagatacttataggtttgtaacatcccaggcaattgcttgtcaa
aaacatacacctccagcacctaaagaagatccccttaaaaaatacactttttgggaagtaaatttaaaggaaaagttttctgcagacctag
atcagtttcctttaggacgcaaatttttactacaagcaggattaaaggccaaaccaaaatttacattaggaaaacgaaaagctacaccca
ccacctcatctacctctacaactgctaaacgcaaaaaacgtaagctgtaagtattgtatgtatgttgactcagtgttgtttgttgtttatatgtc
tgtatgtgcttgtatgtgcttgtaaatattacgttgtatgtgtgtttgtatgtatggtataataaacatgtgtgtatgtgtttttcactgcttgtgtaa
ctattgtgtcatgcaacataaataaacttattgtttcaacacctactaattgtgttgtggttattcattgtatataaactatatttgctacatcctgt
ttttgttttatatatactatattttgtagcgccagcggccattttgtagcttcaaccgaattcggttgcatgctttttggcacaaaatgtgttttttt
aaatagttctatgtcagcaactatagtttaaacttgtacgtttcctgcttgccatgcgtgccaaatccctgttttcctgacctgcactgcttgcc
aaccattccattgttttttacactgcactatgtgcaactactgaatcactatgtacattgtgtcatataaaataaatcactatgcgccaacgcc
ttacataccgctgttaggcacatatttttggcttgttttaactaccctaattgcatatttggcataaggtttaaacttctaaggccaactaaatgt
caccctagttcatacatgaactgtgtaaaggttagtcatacattgttcatttgtaaaactacacatgggtgtgtgcaaaccgttttgggttac
acatttacaagcaacttatataataatactaa
HPV16 E6
(SEQ ID NO: 4)
atgcaccaaaagagaactgcaatgtttcaggacccacaggagcgacccagaaagttaccacagttatgcacagagctgcaaacaact
atacatgatataatattagaatgtgtgtactgcaagcaacagttactgcgacgtgaggtatatgactttgcttttcgggatttatgcatagtat
atagagatgggaatccatatgctgtatgtgataaatgtttaaagttttattctaaaattagtgagtatagacattattgttatagtttgtatggaa
caacattagaacagcaatacaacaaaccgttgtgtgatttgttaattaggtgtattaactgtcaaaagccactgtgtcctgaagaaaagca
aagacatctggacaaaaagcaaagattccataatataaggggtcggtggaccggtcgatgtatgtcttgttgcagatcatcaagaacac
gtagagaaacccagctgtaa
HPV16 E7
(SEQ ID NO: 5)
atgcatggagatacacctacattgcatgaatatatgttagatttgcaaccagagacaactgatctctactgttatgagcaattaaatgacag
ctcagaggaggaggatgaaatagatggtccagctggacaagcagaaccggacagagcccattacaatattgtaaccttttgttgcaag
tgtgacttacgcttcggttgtgcgtacaaagcacacacgtagacattcgtactttggaagacctgttaatgggcacactaggaattgtgtg
ccccatctgttctcagaaaccataa
HPV18 E7
(SEQ ID NO: 6)
atgcatggacctaaggcaacattgcaagacattgtattgcatttagagccccaaaatgaaattccggttgaccttctatgtcacgagcaat
taagcgactcagaggaagaaaacgatgaaatagatggagttaatcatcaacatttaccagcccgacgagccgaaccacaacgtcaca
caatgttgtgtatgtgttgtaagtgtgaagccagaattgagctagtagtagaaagctcagcagacgaccttcgagcattccagcagctgt
ttctgaacaccctgtcctttgtgtgtccgtggtgtgcatcccagcagtaa
Homo sapiens p16/CDKN2A (CDKN2A) (which corresponds to nucleotides 188263-188596
of AB060808.1)
(SEQ ID NO: 7)
acaaattctcagatcatcagtcctcacctgagggaccttccgcggcatctatgcgggcatggttactgcctctggtgccccccgcagcc
gcgcgcaggtaccgtgcgacatcgcgatggcccagctcctcagccaggtccacgggcagacggccccaggcategcgcacgtcc
agccgcgccccggcccggtgcagcaccaccagcgtgtccaggaagccctcccgggcagcgtcgtgcacgggtcgggtgagagtg
gcggggtcggcgcagttgggctccgcgccgtggagcagcagcagctccgccactcgggcgctgcccatcatcatgac
Homo sapiens Tumor Suppressor Protein (P53) (AH007665.2) (nucleotides 1-3423)
(SEQ ID NO: 8)
gagtgcttgggttgtggtgaaacattggaagagagaatgtgaagcagccattcttttcctgctccacaggaagccgagctgtctcagac
actggcatggtgttgggggagggggttccttctctgcaggcccaggtgacccagggttggaagtgtctcatgctggatccccacttttc
ctcttgcagcagccagactgccttccgggtcactgccatggaggagccgcagtcagatcctagegtcgagccccctctgagtcagga
aacattttcagacctatggaaactgtgagtggatccattggaagggcaggcccaccaccccgaccccaaccccagccccctagcaga
gacctgtgggaagcgaaaattccatgggactgactttctgctcttgtctttcagacttcctgaaaacaacgttctggtaaggacaagggtt
gggctggggacctggagggctggggacctggagggctggggggctggggggctgaggacctggtcctctgactgctcttttcaccc
atctacagtcccccttgccgtcccaagcaatggatgatttgatgctgtccccggacgatattgaacaatggttcactgaagacccaggtc
cagatgaagctcccagaatgccagaggctgctccccgcgtggcccctgcaccagcagctcctacaccggeggcccctgcaccagc
cccctectggcccctgtcatcttctgtcccttcccagaaaacctaccagggcagctacggtttccgtctgggcttcttgcattctgggaca
gccaagtctgtgacttgcacggtcagttgccctgaggggctggcttccatgagacttcaatgcctggccgtatccccctgcatttcttttgt
ttggaactttgggattcctcttcaccctttggcttcctgtcagtgtttttttatagtttacccacttaatgtgtgatctctgactcctgtcccaaag
ttgaatattccccccttgaatttgggcttttatccatcccatcacaccctcagcatctctcctggggatgcagaacttttctttttcttcatccac
gtgtattccttggcttttgaaaataagctcctgaccaggcttggtggctcacacctgcaatcccagcactctcaaagaggccaaggcag
gcagatcacctgagcccaggagttcaagaccagcctgggtaacatgatgaaacctcgtctctacaaaaaaatacaaaaaattagccag
gcatggtggtgcacacctatagtcccagccactcaggaggctgaggtgggaagatcacttgaggccaggagatggaggctgcagtg
agctgtgatcacaccactgtgctccagcctgagtgacagagcaagaccctatctcaaaaaaaaaaaaaaagaaaagctcctgaggtgt
agacgccaactctctctagctcgctagtgggttgcaggaggtgcttacacatgtttgtttctttgctgccgtcttccagttgctttatctgttca
cttgtgccctgactttcaactctgtctccttcctcttcctacagtactcccctgccctcaacaagatgttttgccaactggccaagacctgcc
ctgtgcagctgtgggttgattccacacccccgcccggcacccgcgtccgcgccatggccatctacaagcagtcacagcacatgacgg
aggttgtgaggcgctgcccccaccatgagcgctgctcagatagcgatggtgagcagctggggctggagagacgacagggctggtt
gcccagggtccccaggcctctgattcctcactgattgctcttaggtctggcccctcctcagcatcttatccgagtggaaggaaatttgcgt
gtggagtatttggatgacagaaacacttttcgacatagtgtggtggtgccctatgagccgcctgaggtctggtttgcaactggggtctctg
ggaggaggggttaagggtggttgtcagtggccctccgggtgagcagtaggggggctttctcctgctgcttatttgacctccctataacc
ccatgagatgtgcaaagtaaatgggtttaactattgcacagttgaaaaaactgaagcttacagaggctaagggcctcccctgcttggctg
ggcgcagtggctcatgcctgtaatcccagcactttgggaggccaaggcaggcggatcacgaggttgggagatcgagaccatcctgg
ctaacggtgaaaccccgtctctactgaaaaatacaaaaaaaaattagccgggcgtggtgctgggcacctgtagtcccagctactcggg
aggctgaggaaggagaatggcgtgaacctgggcggtggagcttgcagtgagctgagatcacgccactgcactccagcctgggcga
cagagcgagattccatctcaaaaaaaaaaaaaaaaggcctcccctgcttgccacaggtctccccaaggcgcactggcctcatcttggg
cctgtgttatctcctaggttggctctgactgtaccaccatccactacaactacatgtgtaacagttcctgcatgggcggcatgaaccggag
gcccatcctcaccatcatcacactggaagactccaggtcaggagccacttgccaccctgcacactggcctgctgtgccccagcctctg
cttgcctctgacccctgggcccacctcttaccgatttcttccatactactacccatccacctctcatcacatccccggggggaatctcctt
actgctcccactcagttttcttttctctggctttgggacctcttaacctgtggcttctcctccacctacctggagctggagcttaggctccag
aaaggacaagggtggttgggagtagatggagcctggttttttaaatgggacaggtaggacctgatttccttactgcctcttgcttctctttt
cctatcctgagtagtggtaatctactgggacggaacagctttgaggtgcgtgtttgtgcctgtcctgggagagaccggcgcacagaga
aagagaatctccgcaagaaaggggagcctcaccacgagctgcccccagggagcactaagcgaggtaagcaagcaggacaagaa
gcggtggaggagaccaagggtgcagttatgcctcagattcacttttatcacctttccttgcctetttcctagcactgcccaacaacaccag
ctcctctccccagccaaagaagaaaccactggatggagaatatttcacccttcaggtactaagtcttgggacctcttatcaagtggaaag
tttccagtctaacactcaaaatgccgttttcttcttgactgttttacctgcaattggggcatttgccatcagggggcagtgatgcctcaaaga
caatggctcctggttgtagctaa
Homo sapiens Phosphatidylinositol-4,5-bisphosphate 3-kinase Catalytic Subunit Alpha
(PIK3CA) (NM_006218.4)
(SEQ ID NO: 9)
agttccggtgccgccgctgcggccgctgaggtgtcgggctgctgctgccgcggccgctgggactggggctggggccgccggcga
ggcagggctcgggcccggccgggcagctccggagcgggggggagaggggccgggaggcgggggccgtgccgcccgctctc
ctctccctcggcgccgccgccgccgcccgcggggctgggacccgatgcggttagagccgcggagcctggaagagccccgagcgt
ttctgctttgggacaaccatacatctaattccttaaagtagttttatatgtaaaacttgcaaagaatcagaacaatgcctccacgaccatcat
caggtgaactgtggggcatccacttgatgcccccaagaatcctagtagaatgtttactaccaaatggaatgatagtgactttagaatgcct
ccgtgaggctacattaataaccataaagcatgaactatttaaagaagcaagaaaataccccctccatcaacttcttcaagatgaatcttctt
acattttogtaagtgttactcaagaagcagaaagggaagaattttttgatgaaacaagacgactttgtgaccttcggctttttcaacccttttt
aaaagtaattgaaccagtaggcaaccgtgaagaaaagatcctcaatcgagaaattggttttgctatcggcatgccagtgtgtgaatttga
tatggttaaagatccagaagtacaggacttccgaagaaatattctgaacgtttgtaaagaagctgtggatcttagggacctcaattcacct
catagtagagcaatgtatgtctatcctccaaatgtagaatcttcaccagaattgccaaagcacatatataataaattagataaagggcaaa
taatagtggtgatctgggtaatagtttctccaaataatgacaagcagaagtatactctgaaaatcaaccatgactgtgtaccagaacaagt
aattgctgaagcaatcaggaaaaaaactcgaagtatgttgctatcctctgaacaactaaaactctgtgttttagaatatcagggcaagtat
attttaaaagtgtgtggatgtgatgaatacttcctagaaaaatatcctctgagtcagtataagtatataagaagctgtataatgcttgggagg
atgcccaatttgatgttgatggctaaagaaagcctttattctcaactgccaatggactgttttacaatgccatcttattccagacgcatttcca
cagctacaccatatatgaatggagaaacatctacaaaatccctttgggttataaatagtgcactcagaataaaaattctttgtgcaacctac
gtgaatgtaaatattcgagacattgataagatctatgttcgaacaggtatctaccatggaggagaacccttatgtgacaatgtgaacactc
aaagagtaccttgttccaatcccaggtggaatgaatggctgaattatgatatatacattcctgatcttcctcgtgctgctcgactttgcctttc
catttgctctgttaaaggccgaaagggtgctaaagaggaacactgtccattggcatggggaaatataaacttgtttgattacacagacac
tctagtatctggaaaaatggctttgaatctttggccagtacctcatggattagaagatttgctgaaccctattggtgttactggatcaaatcc
aaataaagaaactccatgcttagagttggagtttgactggttcagcagtgtggtaaagttcccagatatgtcagtgattgaagagcatgcc
aattggtctgtatcccgagaagcaggatttagctattcccacgcaggactgagtaacagactagctagagacaatgaattaagggaaaa
tgacaaagaacagctcaaagcaatttctacacgagatcctctctctgaaatcactgagcaggagaaagattttctatggagtcacagaca
ctattgtgtaactatccccgaaattctacccaaattgcttctgtctgttaaatggaattctagagatgaagtagcccagatgtattgcttggta
aaagattggcctccaatcaaacctgaacaggctatggaacttctggactgtaattacccagatcctatggttcgaggttttgctgttcggtg
cttggaaaaatatttaacagatgacaaactttctcagtatttaattcagctagtacaggtcctaaaatatgaacaatatttggataacttgctt
gtgagatttttactgaagaaagcattgactaatcaaaggattgggcactttttcttttggcatttaaaatctgagatgcacaataaaacagtta
gccagaggtttggcctgcttttggagtcctattgtcgtgcatgtgggatgtatttgaagcacctgaataggcaagtcgaggcaatggaaa
agctcattaacttaactgacattctcaaacaggagaagaaggatgaaacacaaaaggtacagatgaagtttttagttgagcaaatgagg
cgaccagatttcatggatgctctacagggctttctgtctcctctaaaccctgctcatcaactaggaaacctcaggcttgaagagtgtcgaa
ttatgtcctctgcaaaaaggccactgtggttgaattgggagaacccagacatcatgtcagagttactgtttcagaacaatgagatcatcttt
aaaaatggggatgatttacggcaagatatgctaacacttcaaattattcgtattatggaaaatatctggcaaaatcaaggtcttgatcttcg
aatgttaccttatggttgtctgtcaatcggtgactgtgtgggacttattgaggtggtgcgaaattctcacactattatgcaaattcagtgcaa
aggcggcttgaaaggtgcactgcagttcaacagccacacactacatcagtggctcaaagacaagaacaaaggagaaatatatgatgc
agccattgacctgtttacacgttcatgtgctggatactgtgtagctaccttcattttgggaattggagatcgtcacaatagtaacatcatggt
gaaagacgatggacaactgtttcatatagattttggacactttttggatcacaagaagaaaaaatttggttataaacgagaacgtgtgcca
tttgttttgacacaggatttcttaatagtgattagtaaaggagcccaagaatgcacaaagacaagagaatttgagaggtttcaggagatgt
gttacaaggcttatctagctattcgacagcatgccaatctcttcataaatcttttctcaatgatgcttggctctggaatgccagaactacaatc
ttttgatgacattgcatacattcgaaagaccctagccttagataaaactgagcaagaggctttggagtatttcatgaaacaaatgaatgatg
cacatcatggtggctggacaacaaaaatggattggatcttccacacaattaaacagcatgcattgaactgaaaagataactgagaaaat
gaaagctcactctggattccacactgcactgttaataactctcagcaggcaaagaccgattgcataggaattgcacaatccatgaacag
cattagaatttacagcaagaacagaaataaaatactatataatttaaataatgtaaacgcaaacagggtttgatagcacttaaactagttca
tttcaaaattaagctttagaataatgcgcaatttcatgttatgccttaagtccaaaaaggtaaactttgaagattgtttgtatctttttttaaaaaa
caaaacaaaacaaaaatccccaaaatatatagaaatgatggagaaggaaaaagtgatggttttttttgtcttgcaaatgttctatgttttgaa
atgtggacacaacaaaggctgttattgcattaggtgtaagtaaactggagtttatgttaaattacattgattggaaaagaatgaaaatttctt
atttttccattgctgttcaatttatagtttgaagtgggtttttgactgcttgtttaatgaagaaaaatgcttggggtggaagggactcttgagatt
tcaccagagacttttttttttaataaatcaaaccttttgatgatttgaggttttatctgcagttttggaagcagtcacaaatgagacctgttata
aggtggtatttttttttttcttctggacagtatttaaaggatcttattcttatttcccagggaaattctgggctoccacaaagtaaaaaaaaaaa
aaaatcatagaaaaagaatgagcaggaatagttcttattccagaattgtacagtattcaccttaagttgattttttttctccttctgcaattgaa
ctgaatacatttttcatgcatgttttccagaaaatagaagtattaatgttattaaaaagattattttttttattaaaggctatttatattatagaaact
atcattaatatatattctttatttacatgatctgtcccatagtcatgcattgttttgcaccccaaattttttattgttcatagcagcatggtcagcttt
cttcttgatctatagatgaggctcaggcactatcccatttataccaataaccagtgtataactacttaaggaaaacataaaaacttcatcttct
ttccttttatttcttatgtgaatctcccgtcttccattctcttttataattgagaatgtctcaatcatatgaaattagttaccagaattaacacaattt
agactatcttcctgattccttaaacccctttactgaagtatactcatgaataatactttaaaatatgggggaatagaaaccatgaactttttac
ctttttaaactatttatccatatctccaaagtagaacattaaaccattttaagatatgtctcattcccaagtagtcagagctcactctccaacttt
attaaatactatttgagcacaggacacattcttaaacattttgaaaaacattaacccaagatgtagaggctactgctagtcgtcattctagaa
tctgatattttactctgtatttgaaatgaatgattaatgtcctaggaaattagctttagcagatgtccaggtgccacatcaaaaaagtgcaata
attattgacagttttttagataggcatattattggaaaacaactttataaagagtgaacattgtatactctagtaaaacagcatcactttaaaaa
tattcatttatgaaatctgttacctatagttgaagtcttgagtagtgaacaagggactctaataccaatactcttaatatctggctattttagatc
ccttaaagggcataattattggaaatttaggtatttcactaaagcatgtatataatattgccaaaagaaaagtaaatttgaagattaagggaa
cttacttctgcaaactgtcttgcgatagttaagcagaatttaaactctgttttaagcaggaaaccagaaagattattttgcagttgtagaagat
ttcataacttattaaaacttattaacattttgtgttgtttagatataggcagttgatacatactaacatcccagccttttcaatatcagggttaaat
tataggaaaactcagtaaaatggtacaaatctgaaagtttgatggtagaaactgaagatttaacagagaactgtgttttacccgagtgcca
aaaatgctgtgagcctccttgcacaaaatttataccacttttgcatttttatctatcagtccagatagttgtctcccctccttctcccaggacct
ctccaccattaaaatgcacaaaccacatggccgatttcaccatttacatttattttcaaaagttactacaaccaaattaattctattagaagaa
atgtagacaaattctataaagactatagattgtgacctaagaaagaaatgaggcaaagaaccaaacattgaattaaatgctacatgggtg
actaagatctgtttcaagtcagtgataatatagccacttctgggtacttcagtatcagagatcagttctcgtggtttagacagttcctatctat
agctgactatccttgtccttgaatatggtgtaactgactattggctctacagttttattgggccacttaagaaatatttccttgaataattattttg
agaaaaagtctaaaagtaataaaaataattttaaacacactgtagtaagaaatgactgttggaaaattatgctttcactttctaccatattctc
agctatacaaaaccatttattttgaagatttttagactactgttaatttgaaatctgttactcttattgtggaatttgtttttttaaaaaagatgtttct
aattggatttttaaaagaagaatggaatttggttgctattttacaatagaacctaagctttttgtggttcttagtgtcctatgtaaaacttagtgt
caaagtaatcaactttgagattttcccttctattctgctttatattaaaagcccattagaaaatgggaacctggtgaatatataatgaattgtaa
aatattttaatgtgtaactttttcaactgtgaaactgacttgattttttgatgaaaacagctgctgataaagtattttgtgtaaagtgtagttcttat
taatcaggaaaatgatgacttgattagactgtatatgccctcttggattttattttaaatggattggtgactttcacataggtaaaacacagtc
catctgtattcttttttccatcaaaaatcgagtgatttggaattataaaaaaattgtgagcagcctatttgaaaggcatcatggaaatttcaca
gcacaataacacggatttgttttttcttaatgatgtaaatccgtttaattcatactttgatcaatagcccatgcttgccaactctgaagaaattta
atttccagcagtattttaaagctagcctgttaactttttctgaatatttaaagttcctcttttttctatgtctgcacaaactgcagacctgggctg
gacccacatactcaagagtccaccttaagaaattattttgatgtccaagacatcactaaaatatttaagtttaaagataatatgtggtgttaat
agattgtggtgcttttactatttaaagacaactttcatacttcagatgtttttgagaagaggggaatgtgaggggagggggcagaacagg
gaggagttgtttgaatgaattacattctttatatccatcctgctcatttggggcatgtctttaagagaaggctgaaagttgtgagagtatattg
tataccgtaagagaatcaactcttcatcatggatgggattgtgaaggctgaactataaaattcagcattgacagcatcctcaattaataatt
cttggtgacagaataatacagctgggctgttttttaaaatataaacaataccatttttaattattacattaaaaattgtaaatatatctatgtgcc
atggcctgggagcctgctttcttttttcataaaaattatttttactgtatgaaaagatcatggggtttagctcaaaatatctgtggtcctgataa
aattggattggtaactctacctcagaaggaaaatgggaaaaaaaaatagatgagtcacaattcaatacttcaagctcagaaactgtgca
gatcactgaattttagatttataaagtcagagttggcatgccttgtttttaatgatatggaagaccttaagaaaaaaacttggctgaagtttaa
tcgttggtccagccatttgaaaaaggcaatagtttgaggaggttcccgaattcggcatttgaaattcattttgttctctcttcttcattattagtg
catttggtgtgtgtatacttgcacacaattctgtttgtgtacacactgcttgcttagccctagtcaagaggcatcttttataaaaggtgtaaag
aaatatcaaggttctaaaattcggaagagtttagaatttattaggagtttcccaagttgggatgttagtctttaaataaacttcatgcacctatt
ccacttaaggttttgcacctcctttttattagtgcagtgccatttcttctgcttgattttaggtatgttaatattccagccttgctagttagcataaa
gtgacaggtgtgagccatgaggaaattttctgacttaatttgtacacaactacatataagagttttagtggaggaaaaaaattagtcccttg
tgcgtatacagtagttaggtaaatgatttttctaccaacagtatactccattcctcatgtaggtaagtacagaaaaggtttttaaatgtatttttt
tagccagttaaagtctatgaatctatctgcaaccttatttaatctgtcactataataattttgtggttatgctaagaaccatgtatacttttaggta
ttcttatttttgtcaatttttctaggttggcaaggaggcagaaaaccttcattgtttcatattaaaatataattagactaaacttaattctagtatg
aatttccaaaatcattatctatttatttcatttttatttaattttgtttttatttcatttttaaaagtcccttgttcaatttaacttatgttcctaagagagg
ttggagaacttggccttcatctgatttcaaaaatgttttgagtttcaaatgaagttaatggtttcagtgtgattcagtcctcagacctaattggg
ttgaataaaatctaaaagaatatacccttttggagcataacattttaataccttggggaatgtggcactaccaaaagaagactactaacac
gtcagatgttcacctggaagctttatcaagaaattcgaaccacccttttggccccattaattgtagcaagtttatttctctatattttgtcattca
gtgaattgaagtcctgtggtatactgcattcattagaagaaaaacgtttttaatgtccttttaatgatggcccagaaagcatttgacacagca
agatgcatgtgttactatattgagaatatagaataataacagtatcactaaatttaagacctcttcccagtcttgctgttcctagcaagaagtt
tggcctgtgactgcacttactgtttatgctcatcagaaactgtcaatgtctgcttttctttaactctgcagtctgtaacatcacgctgtttattaa
aaaaaaaaagaaaaatta
Homo sapiens HRAS Proto-Oncogene, GTPase (HRAS) Transcript Variant 4 (MZ068328.1)
(SEQ ID NO: 10)
gcagtcgcgcctgtgaacggattcctaccggaagcaggtggtcattgatggggagacgtgcctgttggacatcctggataccgccgg
ccaggaggagtacagcgccatgcgggaccagtacatgcgcaccggggagggcttcctgtgtgtgtttgccatcaacaacaccaagt
cttttgaggacatccaccagtacagggagcagatcaaacgggtgaaggactcggatgacgtgcccatggtgctggtggggaacaag
tgtgacctggctgcacgcactgtggaatctcggcaggctcaggacctcgcccgaagctacggcatcccctacatcgagacctcggcc
aagacccggcagggcagccgctctggctctagctccagctccgggaccctctgggaccccccgggacccatgtgacccagcggcc
cctcgcgctggagtggaggatgccttctacacgttggtgcgtgagatccggcagcacaagctgcggaagctgaaccctcctgatgag
agtggccccggctgcatgagctgcaagtgtgtgctctcctgacgcaggtgagggggactcccagggcggccgccacgcccaccgg
atgaccccggctccccgcccctgccggtctcctggcctgcggtcagcagcctcccttgtgccccgcccagcacaagctcaggacatg
gaggtgccggatgcaggaaggaggtgcagacggaaggaggaggaaggaaggacggaagcaaggaaggaaggaagggctgct
ggagcccagtcaccccgggaccgtgggccgaggtgactgcagaccctcccagggaggctgtgcacagactgtcttgaacatccca
aatgccaccggaaccccagcccttagctcccctcccaggctctgtgggcccttgtcgggcacagatgggatcacagtaaattattggat
ggtcttga
Homo sapiens HRAS Proto-Oncogene, GTPase (HRAS) Transcript Variant 1
(NM_005343.4)
(SEQ ID NO: 11)
aggcccgcccgagtctccgccgcccgtgccctgcgcccgcaacccgagccgcacccgccgcggacggagcccatgcgcggggc
gaaccgcgcgcccccgcccccgccccgccccggcctcggccccggccctggccccgggggcagtcgcgcctgtgaacggtggg
gcaggagaccctgtaggaggaccccgggccgcaggcccctgaggagcgatgacggaatataagctggtggtggtgggcgccgg
cggtgtgggcaagagtgcgctgaccatccagctgatccagaaccattttgtggacgaatacgaccccactatagaggattcctaccgg
aagcaggtggtcattgatggggagacgtgcctgttggacatcctggataccgccggccaggaggagtacagcgccatgcgggacc
agtacatgcgcaccggggagggcttcctgtgtgtgtttgccatcaacaacaccaagtcttttgaggacatccaccagtacagggagca
gatcaaacgggtgaaggactcggatgacgtgcccatggtgctggtggggaacaagtgtgacctggctgcacgcactgtggaatctcg
gcaggctcaggacctcgcccgaagctacggcatcccctacatcgagacctcggccaagacccggcagggagtggaggatgccttc
tacacgttggtgcgtgagatccggcagcacaagctgcggaagctgaaccctcctgatgagagtggccccggctgcatgagctgcaa
gtgtgtgctctcctgacgcagcacaagctcaggacatggaggtgccggatgcaggaaggaggtgcagacggaaggaggaggaag
gaaggacggaagcaaggaaggaaggaagggctgctggagcccagtcaccccgggaccgtgggccgaggtgactgcagaccctc
ccagggaggctgtgcacagactgtcttgaacatcccaaatgccaccggaaccccagcccttagctcccctcccaggcctctgtgggc
ccttgtcgggcacagatgggatcacagtaaattattggatggtcttga
Homo sapiens Neuroblastoma RAS Viral (V-RAS) Oncogene Homolog (NRAS)
(NM_005343.4)
(SEQ ID NO: 12)
aggcccgcccgagtctccgccgcccgtgccctgcgcccgcaacccgagccgcacccgccgcggacggagcccatgcgcggggc
gaaccgcgcgcccccgcccccgccccgccccggcctcggccccggccctggccccgggggcagtcgcgcctgtgaacggtggg
gcaggagaccctgtaggaggaccccgggccgcaggcccctgaggagcgatgacggaatataagctggtggtggtgggcgccgg
cggtgtgggcaagagtgcgctgaccatccagctgatccagaaccattttgtggacgaatacgaccccactatagaggattcctaccgg
aagcaggtggtcattgatggggagacgtgcctgttggacatcctggataccgccggccaggaggagtacagcgccatgcgggacc
agtacatgcgcaccggggagggcttcctgtgtgtgtttgccatcaacaacaccaagtcttttgaggacatccaccagtacagggagca
gatcaaacgggtgaaggactcggatgacgtgcccatggtgctggtggggaacaagtgtgacctggctgcacgcactgtggaatctcg
gcaggctcaggacctcgcccgaagctacggcatcccctacatcgagacctcggccaagacccggcagggagtggaggatgccttc
tacacgttggtgcgtgagatccggcagcacaagctgcggaagctgaaccctcctgatgagagtggccccggctgcatgagctgcaa
gtgtgtgctctcctgacgcagcacaagctcaggacatggaggtgccggatgcaggaaggaggtgcagacggaaggaggaggaag
gaaggacggaagcaaggaaggaaggaagggctgctggagcccagtcaccccgggaccgtgggccgaggtgactgcagaccctc
ccagggaggctgtgcacagactgtcttgaacatcccaaatgccaccggaaccccagcccttagctcccctcccaggcctctgtgggc
ccttgtcgggcacagatgggatcacagtaaattattggatggtcttga
Homo sapiens RAS Family Small GTP Binding Protein K-RAS2 (KRAS) (AF493917.1)
(SEQ ID NO: 13)
atgactgaatataaacttgtggtagttggagctggtggcgtaggcaagagtgccttgacgatacagctaattcagaatcattttgtggacg
aatatgatccaacaatagaggattcctacaggaagcaagtagtaattgatggagaaacctgtctcttggatattctcgacacagcaggtc
aagaggagtacagtgcaatgagggaccagtacatgaggactggggagggctttctttgtgtatttgccataaataatactaaatcatttg
aagatattcaccattatagagaacaaattaaaagagttaaggactctgaagatgtacctatggtcctagtaggaaataaatgtgatttgcct
tctagaacagtagacacaaaacaggctcaggacttagcaagaagttatggaattccttttattgaaacatcagcaaagacaagacaggg
tgttgatgatgccttctatacattagttcgagaaattcgaaaacataaagaaaagatgagcaaagacggtaaaaagaagaaaaagaagt
caaagacaaagtgtgtaattatgtaa
Homo sapiens F-BOX and WD Repeat Domain Containing 7 (FBXW7) Transcript Variant 1
(NM_033632.3)
(SEQ ID NO: 14)
taccgcgccggagccttccgcagctgccgcttcagtccgaaggaggaagggaaccaacccactttctcggcgccgcggctcttttcta
aaagtaatgtgaaaacctttgcatcttctgatagtctagccaaggtccaagaagtagcaagctggcttttggaaatgaatcaggaactgct
ctctgtgggcagcaaaagacgacgaactggaggctctctgagaggtaacccttcctcaagccaggtagatgaagaacagatgaatcg
tgtggtagaggaggaacagcaacagcaactcagacaacaagaggaggagcacactgcaaggaatggtgaagttgttggagtagaa
cctagacctggaggccaaaatgattcccagcaaggacagttggaagaaaacaataatagatttatttcggtagatgaggactcctcagg
aaaccaagaagaacaagaggaagatgaagaacatgctggtgaacaagatgaggaggatgaggaggaggaggagatggaccagg
agagtgacgattttgatcagtctgatgatagtagcagagaagatgaacatacacatactaacagtgtcacgaactccagtagtattgtgg
acctgcccgttcaccaactctcctccccattctatacaaaaacaacaaaaatgaaaagaaagttggaccatggttctgaggtccgctcttt
ttctttgggaaagaaaccatgcaaagtctcagaatatacaagtaccactgggcttgtaccatgttcagcaacaccaacaacttttgggga
cctcagagcagccaatggccaagggcaacaacgacgccgaattacatctgtccagccacctacaggcctccaggaatggctaaaaa
tgtttcagagctggagtggaccagagaaattgcttgctttagatgaactcattgatagttgtgaaccaacacaagtaaaacatatgatgca
agtgatagaaccccagtttcaacgagacttcatttcattgctccctaaagagttggcactctatgtgctttcattcctggaacccaaagacc
tgctacaagcagctcagacatgtcgctactggagaattttggctgaagacaaccttctctggagagagaaatgcaaagaagaggggat
tgatgaaccattgcacatcaagagaagaaaagtaataaaaccaggtttcatacacagtccatggaaaagtgcatacatcagacagcac
agaattgatactaactggaggcgaggagaactcaaatctcctaaggtgctgaaaggacatgatgatcatgtgatcacatgcttacagttt
tgtggtaaccgaatagttagtggttctgatgacaacactttaaaagtttggtcagcagtcacaggcaaatgtctgagaacattagtgggac
atacaggtggagtatggtcatcacaaatgagagacaacatcatcattagtggatctacagatcggacactcaaagtgtggaatgcaga
gactggagaatgtatacacaccttatatgggcatacttccactgtgcgttgtatgcatcttcatgaaaaaagagttgttagcggttctcgag
atgccactcttagggtttgggatattgagacaggccagtgtttacatgttttgatgggtcatgttgcagcagtccgctgtgttcaatatgatg
gcaggagggttgttagtggagcatatgattttatggtaaaggtgtgggatccagagactgaaacctgtctacacacgttgcaggggcat
actaatagagtctattcattacagtttgatggtatccatgtggtgagtggatctcttgatacatcaatccgtgtttgggatgtggagacaggg
aattgcattcacacgttaacagggcaccagtcgttaacaagtggaatggaactcaaagacaatattcttgtctctgggaatgcagattcta
cagttaaaatctgggatatcaaaacaggacagtgtttacaaacattgcaaggtcccaacaagcatcagagtgctgtgacctgtttacagt
tcaacaagaactttgtaattaccagctcagatgatggaactgtaaaactatgggacttgaaaacgggtgaatttattcgaaacctagtcac
attggagagtggggggagtgggggagttgtgtggcggatcagagcctcaaacacaaagctggtgtgtgcagttgggagtcggaatg
ggactgaagaaaccaagctgctggtgctggactttgatgtggacatgaagtgaagagcagaaagatgaatttgtccaattgtgtagacg
atatactccctgcccttccccctgcaaaaagaaaaaaagaaaagaaaaagaaaaaaatcccttgttctcagtggtgcaggatgttggctt
ggggcaacagattgaaaagacctacagactaagaaggaaaagaagaagagatgacaaaccataactgacaagagaggcgtctgct
gtctcatcacataaaaggcttcacttttgactgagggcagctttgcaaaatgagactttctaaatcaaaccaggtgcaattatttctttattttc
ttctccagtggtcattgggcagtgttaatgctgaaacatcattacagattctgctagcctgttcttttaccactgacagctagacacctagaa
aggaactgcaataatatcaaaacaagtactggttgactttctaattagagagcatctgcaacaaaaagtcatttttctggagtggaaaagc
ttaaaaaaattactgtgaattgtttttgtacagttatcatgaaaagcttttttttttttttttttgccaaccattgccaatgtcaatcaatcacagtatt
agcctctgttaatctatttactgttgcttccatatacattcttcaatgcatatgttgctcaaaggtggcaagttgtcctgggttctgtgagtcctg
agatggatttaattcttgatgctggtgctagaagtaggtcttcaaatatgggattgttgtcccaaccctgtactgtactcccagtggccaaa
cttatttatgctgctaaatgaaagaaagaaaaaagcaaattatttttttttattttttttctgctgtgacgttttagtcccagactgaattccaaatt
tgctctagtttggttatggaaaaaagactttttgccactgaaacttgagccatctgtgcctctaagaggctgagaatggaagagtttcagat
aataaagagtgaagtttgcctgcaagtaaagaattgagagtgtgtgcaaagcttattttcttttatctgggcaaaaattaaaacacattcctt
ggaacagagctattacttgcctgttctgtggagaaacttttctttttgagggctgtggtgaatggatgaacgtacatcgtaaaactgacaaa
atattttaaaaatatataaaacacaaaattaaaataaagttgctggtcagtcttagtgttttacagtatttgggaaaacaactgttacagttttat
tgctctgagtaactgacaaagcagaaactattcagtttttgtagtaaaggcgtcacatgcaaacaaacaaaatgaatgaaacagtcaaat
ggtttgcctcattctccaagagccacaactcaagctgaactgtgaaagtggtttaacactgtatcctaggcgatcttttttcctccttctgttt
atttttttgtttgttttatttatagtctgatttaaaacaatcagattcaagttggttaattttagttatgtaacaacctgacatgatggaggaaaaca
acctttaaagggattgtgtctatggtttgattcacttagaaattttattttcttataacttaagtgcaataaaatgtgttttttcatgttaaaaaaaa
aaaaaaaaaaa
Homo sapiens Cyclin D1 (BC023620.2)
(SEQ ID NO: 15)
agcgagcagcagagtccgcacgctccggcgaggggcagaagagcgcgagggagcgcggggcagcagaagcgagagccgag
cgcggacccagccaggacccacagccctccccagctgcccaggaagagccccagccatggaacaccagctcctgtgctgcgaag
tggaaaccatccgccgcgcgtaccccgatgccaacctcctcaacgaccgggtgctgcgggccatgctgaaggcggaggagacctg
cgcgccctcggtgtcctacttcaaatgtgtgcagaaggaggtcctgccgtccatgcggaagatcgtcgccacctggatgctggaggtc
tgcgaggaacagaagtgcgaggaggaggtcttcccgctggccatgaactacctggaccgcttcctgtcgctggagcccgtgaaaaa
gagccgcctgcagctgctgggggccacttgcatgttcgtggcctctaagatgaaggagaccatccccctgacggccgagaagctgtg
catctacaccgacaactccatccggcccgaggagctgctgcaaatggagctgctcctggtgaacaagctcaagtggaacctggccgc
aatgaccccgcacgatttcattgaacacttcctctccaaaatgccagaggcggaggagaacaaacagatcatccgcaaacacgcgca
gaccttcgttgccctctgtgccacagatgtgaagttcatttccaatccgccctccatggtggcagcggggagcgtggtggccgcagtgc
aaggcctgaacctgaggagccccaacaacttcctgtcctactaccgcctcacacgcttcctctccagagtgatcaagtgtgacccaga
ctgcctccgggcctgccaggagcagatcgaagccctgctggagtcaagcctgcgccaggcccagcagaacatggaccccaaggc
cgccgaggaggaggaagaggaggaggaggaggtggacctggcttgcacacccaccgacgtgcgggacgtggacatctgagggc
gccaggcaggcgggcgccaccgccacccgcagcgagggcggagccggccccaggtgctccactgacagtccctcctctccgga
gcattttgataccagaagggaaagcttcattctccttgttgttggttgttttttcctttgctctttcccccttccatctctgacttaagcaaaaga
aaaagattacccaaaaactgtctttaaaagagagagagagaaaaaaaaaatagtatttgcataaccctgagcggtgggggaggaggg
ttgtgctacagatgatagaggattttataccccaataatcaactcgtttttatattaatgtacttgtttctctgttgtaagaataggcattaacac
aaaggaggcgtctcgggagaggattaggttccatcctttacgtgtttaaaaaaaagcataaaaacattttaaaaacatagaaaaattcag
caaaccatttttaaagtagaagagggttttaggtagaaaaacatattcttgtgcttttcctgataaagcacagctgtagtggggttctaggc
atctctgtactttgcttgctcatatgcatgtagtcactttataagtcattgtatgttattatattccgtaggtagatgtgtaacctcttcaccttatt
catggctgaagtcacctcttggttacagtagcgtagcgtggccgtgtgcatgtcctttgcgcctgtgaccaccaccccaacaaaccatc
cagtgacaaaccatccagtggaggtttgtcgggcaccagccagcgtagcagggtcgggaaaggccacctgtcccactcctacgata
cgctactataaagagaagacgaaatagtgacataatatattctatttttatactcttcctatttttgtagtgacctgtttatgagatgctggttttc
tacccaacggccctgcagccagctcacgtccaggttcaacccacagctacttggtttgtgttcttcttcatattctaaaaccattccatttcc
aagcactttcagtccaataggtgtaggaaatagcgctgtttttgttgtgtgtgcagggagggcagttttctaatggaatggtttgggaatat
ccatgtacttgtttgcaagcaggactttgaggcaagtgtgggccactgtggtggcagtggaggtggggtgtttgggaggctgcgtgcc
agtcaagaagaaaaaggtttgcattctcacattgccaggatgataagttcctttccttttctttaaagaagttgaagtttaggaatcctttggt
gccaactggtgtttgaaagtagggacctcagaggtttacctagagaacaggtggtttttaagggttatcttagatgtttcacaccggaagg
tttttaaacactaaaatatataatttatagttaaggctaaaaagtatatttattgcagaggatgttcataaggccagtatgatttataaatgcaat
ctccccttgatttaaacacacagatacacacacacacacacacacacacacaaaccttctgcctttgatgttacagatttaatacagtttatt
tttaaagatagatccttttataggtgagaaaaaaacaatctggaagaaaaaaaccacacaaagacattgattcagcctgtttggcgtttcc
cagagtcatctgattggacaggcatgggtgcaaggaaaattagggtactcaacctaagttcggttccgatgaattcttatcccctgcccc
ttcctttaaaaaacttagtgacaaaatagacaatttgcacatcttggctatgtaattcttgtaatttttatttaggaagtgttgaagggaggtgg
caagagtgtggaggctgacgtgtgagggaggacaggcgggaggaggtgtgaggaggaggctcccgaggggaaggggcggtgc
ccacaccggggacaggccgcagctccattttcttattgcgctgctaccgttgacttccaggcacggtttggaaatattcacatcgcttctg
tgtatctctttcacattgtttgctgctattggaggatcagttttttgttttacaatgtcatatactgccatgtactagttttagttttctcttagaacat
tgtattacagatgccttttttgtagttttttttttttttatgtgatcaattttgacttaatgtgattactgctctattccaaaaaggttgctgtttcacaa
tacctcatgcttcacttagccatggtggacccagcgggcaggttctgcctgctttggcgggcagacacgcgggcgcgatcccacaca
ggctggcgggggccggccccgaggccgcgtgcgtagaaccgcgccggtgtccccagagaccaggctgtgtccctcttctcttccct
gcgcctgtgatgctgggcacttcatctgatcgggggcgtagcatcatagtagtttttacagctgtgttattctttgcgtgtagctatggaagt
tgcataattattattattattattataacaagtgtgtcttacgtgccaccacggcgttgtacctgtaggactctcattcgggatgattggaata
gcttctggaatttgttcaagttttgggtatgtttaatctgttatgtactagtgttctgtttgttattgttttgttaattacaccataatgctaatttaaa
gagactccaaatctcaatgaagccagctcacagtgctgtgtgccccggtcacctagcaagctgccgaaccaaaagaatttgcacccc
gctgcgggcccacgtggttggggccctgccctggcagggtcatcctgtgctcggaggccatctcgggcacaggccaccccgcccc
acccctccagaacacggctcacgcttacctcaaccatcctggctgcggcgtctgtctgaaccacgcgggggccttgagggacgctttg
tctgtcgtgatggggcaagggcacaagtcctggatgttgtgtgtatcgagaggccaaaggctggtggcaagtgcacggggcacagc
ggagtctgtcctgtgacgcgcaagtctgagggtctgggcggcgggcggctgggtctgtgcatttctggttgcaccgcggcgcttccca
gcaccaacatgtaaccggcatgtttccagcagaagacaaaaagacaaacatgaaagtctagaaataaaactggtaaaaccccaaaaa
aaaaaaaaaaaaaaaaaaaaaaa
Homo sapiens Epidermal Growth Factor Receptor (EGFR) Transcript Variant 1
(NM_005228.5)
(SEQ ID NO: 16)
agacgtccgggcagcccccggcgcagcgcggccgcagcagcctccgccccccgcacggtgtgagcgcccgacgcggccgagg
cggccggagtcccgagctagccccggcggccgccgccgcccagaccggacgacaggccacctcgtcggcgtccgcccgagtcc
ccgcctcgccgccaacgccacaaccaccgcgcacggccccctgactccgtccagtattgatcgggagagccggagcgagctcttc
ggggagcagcgatgcgaccctccgggacggccggggcagcgctcctggcgctgctggctgcgctctgcccggcgagtcgggctc
tggaggaaaagaaagtttgccaaggcacgagtaacaagctcacgcagttgggcacttttgaagatcattttctcagcctccagaggatg
ttcaataactgtgaggtggtccttgggaatttggaaattacctatgtgcagaggaattatgatctttccttcttaaagaccatccaggaggtg
gctggttatgtcctcattgccctcaacacagtggagcgaattcctttggaaaacctgcagatcatcagaggaaatatgtactacgaaaatt
cctatgccttagcagtcttatctaactatgatgcaaataaaaccggactgaaggagctgcccatgagaaatttacaggaaatcctgcatg
gcgccgtgcggttcagcaacaaccctgccctgtgcaacgtggagagcatccagtggcgggacatagtcagcagtgactttctcagca
acatgtcgatggacttccagaaccacctgggcagctgccaaaagtgtgatccaagctgtcccaatgggagctgctggggtgcaggag
aggagaactgccagaaactgaccaaaatcatctgtgcccagcagtgctccgggcgctgccgtggcaagtcccccagtgactgctgc
cacaaccagtgtgctgcaggctgcacaggcccccgggagagcgactgcctggtctgccgcaaattccgagacgaagccacgtgca
aggacacctgccccccactcatgctctacaaccccaccacgtaccagatggatgtgaaccccgagggcaaatacagctttggtgcca
cctgcgtgaagaagtgtccccgtaattatgtggtgacagatcacggctcgtgcgtccgagcctgtggggccgacagctatgagatgga
ggaagacggcgtccgcaagtgtaagaagtgcgaagggccttgccgcaaagtgtgtaacggaataggtattggtgaatttaaagactc
actctccataaatgctacgaatattaaacacttcaaaaactgcacctccatcagtggcgatctccacatcctgccggtggcatttaggggt
gactccttcacacatactcctcctctggatccacaggaactggatattctgaaaaccgtaaaggaaatcacagggtttttgctgattcagg
cttggcctgaaaacaggacggacctccatgcctttgagaacctagaaatcatacgcggcaggaccaagcaacatggtcagttttctctt
gcagtcgtcagcctgaacataacatccttgggattacgctccctcaaggagataagtgatggagatgtgataatttcaggaaacaaaaa
tttgtgctatgcaaatacaataaactggaaaaaactgtttgggacctccggtcagaaaaccaaaattataagcaacagaggtgaaaaca
gctgcaaggccacaggccaggtctgccatgccttgtgctcccccgagggctgctggggcccggagcccagggactgcgtctcttgc
cggaatgtcagccgaggcagggaatgcgtggacaagtgcaaccttctggagggtgagccaagggagtttgtggagaactctgagtg
catacagtgccacccagagtgcctgcctcaggccatgaacatcacctgcacaggacggggaccagacaactgtatccagtgtgccc
actacattgacggcccccactgcgtcaagacctgcccggcaggagtcatgggagaaaacaacaccctggtctggaagtacgcagac
gccggccatgtgtgccacctgtgccatccaaactgcacctacggatgcactgggccaggtcttgaaggctgtccaacgaatgggcct
aagatcccgtccatcgccactgggatggtgggggccctcctcttgctgctggtggtggccctggggatcggcctcttcatgcgaaggc
gccacatcgttcggaagcgcacgctgcggaggctgctgcaggagagggagcttgtggagcctcttacacccagtggagaagctccc
aaccaagctctcttgaggatcttgaaggaaactgaattcaaaaagatcaaagtgctgggctccggtgcgttcggcacggtgtataagg
gactctggatcccagaaggtgagaaagttaaaattcccgtcgctatcaaggaattaagagaagcaacatctccgaaagccaacaagg
aaatcctcgatgaagcctacgtgatggccagcgtggacaacccccacgtgtgccgcctgctgggcatctgcctcacctccaccgtgc
agctcatcacgcagctcatgcccttcggctgcctcctggactatgtccgggaacacaaagacaatattggctcccagtacctgctcaac
tggtgtgtgcagatcgcaaagggcatgaactacttggaggaccgtcgcttggtgcaccgcgacctggcagccaggaacgtactggtg
aaaacaccgcagcatgtcaagatcacagattttgggctggccaaactgctgggtgcggaagagaaagaataccatgcagaaggagg
caaagtgcctatcaagtggatggcattggaatcaattttacacagaatctatacccaccagagtgatgtctggagctacggggtgactgt
ttgggagttgatgacctttggatccaagccatatgacggaatccctgccagcgagatctcctccatcctggagaaaggagaacgcctc
cctcagccacccatatgtaccatcgatgtctacatgatcatggtcaagtgctggatgatagacgcagatagtcgcccaaagttccgtga
gttgatcatcgaattctccaaaatggcccgagacccccagcgctaccttgtcattcagggggatgaaagaatgcatttgccaagtcctac
agactccaacttctaccgtgccctgatggatgaagaagacatggacgacgtggtggatgccgacgagtacctcatcccacagcaggg
cttcttcagcagcccctccacgtcacggactcccctcctgagctctctgagtgcaaccagcaacaattccaccgtggcttgcattgatag
aaatgggctgcaaagctgtcccatcaaggaagacagcttcttgcagcgatacagctcagaccccacaggcgccttgactgaggacag
catagacgacaccttcctcccagtgcctgaatacataaaccagtccgttcccaaaaggcccgctggctctgtgcagaatcctgtctatca
caatcagcctctgaaccccgcgcccagcagagacccacactaccaggacccccacagcactgcagtgggcaaccccgagtatctc
aacactgtccagcccacctgtgtcaacagcacattcgacagccctgcccactgggcccagaaaggcagccaccaaattagcctgga
caaccctgactaccagcaggacttctttcccaaggaagccaagccaaatggcatctttaagggctccacagctgaaaatgcagaatac
ctaagggtcgcgccacaaagcagtgaatttattggagcatgaccacggaggatagtatgagccctaaaaatccagactctttogatacc
caggaccaagccacagcaggtcctccatcccaacagccatgcccgcattagctcttagacccacagactggttttgcaacgtttacacc
gactagccaggaagtacttccacctcgggcacattttgggaagttgcattcctttgtcttcaaactgtgaagcatttacagaaacgcatcc
agcaagaatattgtccctttgagcagaaatttatctttcaaagaggtatatttgaaaaaaaaaaaaagtatatgtgaggatttttattgattgg
ggatcttggagtttttcattgtcgctattgatttttacttcaatgggctcttccaacaaggaagaagcttgctggtagcacttgctaccctgag
ttcatccaggcccaactgtgagcaaggagcacaagccacaagtcttccagaggatgcttgattccagtggttctgcttcaaggcttcca
ctgcaaaacactaaagatccaagaaggccttcatggccccagcaggccggatcggtactgtatcaagtcatggcaggtacagtagga
taagccactctgtcccttcctgggcaaagaagaaacggaggggatggaattcttccttagacttacttttgtaaaaatgtccccacggtac
ttactccccactgatggaccagtggtttccagtcatgagcgttagactgacttgtttgtcttccattccattgttttgaaactcagtatgctgcc
cctgtcttgctgtcatgaaatcagcaagagaggatgacacatcaaataataactcggattccagcccacattggattcatcagcatttgga
ccaatagcccacagctgagaatgtggaatacctaaggatagcaccgcttttgttctcgcaaaaacgtatctcctaatttgaggctcagat
gaaatgcatcaggtcctttggggcatagatcagaagactacaaaaatgaagctgctctgaaatctcctttagccatcaccccaaccccc
caaaattagtttgtgttacttatggaagatagttttctccttttacttcacttcaaaagctttttactcaaagagtatatgttccctccaggtcagc
tgcccccaaaccccctccttacgctttgtcacacaaaaagtgtctctgccttgagtcatctattcaagcacttacagctctggccacaaca
gggcattttacaggtgcgaatgacagtagcattatgagtagtgtggaattcaggtagtaaatatgaaactagggtttgaaattgataatgc
tttcacaacatttgcagatgttttagaaggaaaaaagttccttcctaaaataatttctctacaattggaagattggaagattcagctagttagg
agcccaccttttttcctaatctgtgtgtgccctgtaacctgactggttaacagcagtcctttgtaaacagtgttttaaactctcctagtcaatat
ccaccccatccaatttatcaaggaagaaatggttcagaaaatattttcagcctacagttatgttcagtcacacacacatacaaaatgttcctt
ttgcttttaaagtaatttttgactcccagatcagtcagagcccctacagcattgttaagaaagtatttgatttttgtctcaatgaaaataaaact
atattcatttccactctattatgctctcaaatacccctaagcatctatactagcctggtatgggtatgaaagatacaaagataaataaaacat
agtccctgattctaagaaattcacaatttagcaaaggaaatggactcatagatgctaaccttaaaacaacgtgacaaatgccagacagg
acccatcagccaggcactgtgagagcacagagcagggaggttgggtcctgcctgaggagacctggaagggaggcctcacaggag
gatgaccaggtctcagtcagcggggaggtggaaagtgcaggtgcatcaggggcaccctgaccgaggaaacagctgccagaggcc
tccactgctaaagtccacataaggctgaggtcagtcaccctaaacaacctgctccctctaagccaggggatgagcttggagcatccca
caagttccctaaaagttgcagcccccagggggattttgagctatcatctctgcacatgcttagtgagaagactacacaacatttctaagaa
tctgagattttatattgtcagttaaccactttcattattcattcacctcaggacatgcagaaatatttcagtcagaactgggaaacagaagga
cctacattctgctgtcacttatgtgtcaagaagcagatgatcgatgaggcaggtcagttgtaagtgagtcacattgtagcattaaattctag
tatttttgtagtttgaaacagtaacttaataaaagagcaaaagctattctagctttcttcttcatattttaattttccaccataaagtttagttgcta
aattctattaattttaagattgtgcttcccaaaatagttctcacttcatctgtccagggaggcacagttctgtctggtagaagccgcaaagcc
cttagcctcttcacggatctggcgactgtgatgggcaggtcaggagaggagctgcccaaagtcccatgattttcacctaacagccctga
tcagtcagtactcaaagcttggactccatccctgaaggtcttcctgattgatagcctggccttaataccctacagaaagcctgtccattgg
ctgtttcttcctcagtcagttcctggaagaccttaccccatgaccccagcttcagatgtggtctttggaaacagaggtcgaaggaaagtaa
ggagctgagagctcacattcataggtgccgccagccttcgtgcatcttcttgcatcatctctaaggagctcctctaattacaccatgcccg
tcaccccatgagggatcagagaagggatgagtcttctaaactctatattcgctgtgagtccaggttgtaagggggagcactgtggatgc
atcctattgcactccagctgatgacaccaaagcttaggtgtttgctgaaagttcttgatgttgtgacttaccacccctgcctcacaactgca
gacataaggggactatggattgcttagcaggaaaggcactggttctcaagggcggctgcccttgggaatcttctggtcccaaccagaa
agactgtggcttgattttctcaggtgcagcccagccgtagggccttttcagagcaccccctggttattgcaacattcatcaaagtttctaga
acctctggcctaaaggaagggcctggtgggatctacttggcactcgctggggggccaccccccagtgccactctcactaggcctctg
attgcacttgtgtaggatgaagctggtgggtgatgggaactcagcacctcccctcaggcagaaaagaatcatctgtggagcttcaaaa
gaaggggcctggagtctctgcagaccaattcaacccaaatctcgggggctctttcatgattctaatgggcaaccagggttgaaaccctt
atttctagggtcttcagttgtacaagactgtgggtctgtaccagagcccccgtcagagtagaataaaaggctgggtagggtagagattc
ccatgtgcagtggagagaacaatctgcagtcactgataagcctgagacttggctcatttcaaaagcgttcaattcatcctcaccagcagt
tcagctggaaaggggcaaatacccccacctgagctttgaaaacgccctgggaccctctgcattctctaagtaagttatagaaaccagtc
tcttccctcctttgtgagtgagctgctattccacgtaggcaacacctgttgaaattgccctcaatgtctactctgcatttctttcttgtgataag
cacacacttttattgcaacataatgatctgctcacatttccttgcctgggggctgtaaaaccttacagaacagaaatccttgcctctttcacc
agccacacctgccataccaggggtacagctttgtactattgaagacacagacaggatttttaaatgtaaatctatttttgtaactttgttgcg
ggatatagttctctttatgtagcactgaactttgtacaatatatttttagaaactcatttttctactaaaacaaacacagtttactttagagagact
gcaatagaatcaaaatttgaaactgaaatctttgtttaaaagggttaagttgaggcaagaggaaagccctttctctctcttataaaaaggca
caacctcattggggagctaagctaggtcattgtcatggtgaagaagagaagcatcgtttttatatttaggaaattttaaaagatgatggaa
agcacatttagcttggtctgaggcaggttctgttggggcagtgttaatggaaagggctcactgttgttactactagaaaaatccagttgca
tgccatactctcatcatctgccagtgtaaccctgtacatgtaagaaaagcaataacatagcactttgttggtttatatatataatgtgacttca
atgcaaattttatttttatatttacaattgatatgcatttaccagtataaactagacatgtctggagagcctaataatgttcagcacactttggtt
agttcaccaacagtcttaccaagcctgggcccagccaccctagagaagttattcagccctggctgcagtgacatcacctgaggagcttt
taaaagcttgaagcccagctacacctcagaccgattaaacgcaaatctctggggctgaaacccaagcattcgtagtttttaaagctcctg
aggtcattccaatgtgcggccaaagttgagaactactggcctagggattagccacaaggacatggacttggaggcaaattctgcaggt
gtatgtgattctcaggcctagagagctaagacacaaagacctccacatctgtcgctgagagtcaagaacctgaacagagtttccatgaa
ggttctccaagcactagaagggagagtgtctaaacaatggttgaaaagcaaaggaaatataaaacagacacctctttccatttcctaag
gtttctctctttattaagggtggactagtaataaaatataatattcttgctgcttatgcagctgacattgttgccctccctaaagcaaccaagta
gcctttatttcccacagtgaaagaaaacgctggcctatcagttacattacaaaaggcagatttcaagaggattgagtaagtagttggatg
gctttcataaaaacaagaattcaagaagaggattcatgctttaagaaacatttgttatacattcctcacaaattatacctgggataaaaacta
tgtagcaggcagtgtgttttccttccatgtctctctgcactacctgcagtgtgtcctctgaggctgcaagtctgtcctatctgaattcccagc
agaagcactaagaagctccaccctatcacctagcagataaaactatggggaaaacttaaatctgtgcatacatttctggatgcatttactt
atctttaaaaaaaaaggaatcctatgacctgatttggccacaaaaataatcttgctgtacaatacaatctcttggaaattaagagatcctatg
gatttgatgactggtattagaggtgacaatgtaaccgattaacaacagacagcaataacttcgttttagaaacattcaagcaatagctttat
agcttcaacatatggtacgttttaaccttgaaagttttgcaatgatgaaagcagtatttgtacaaatgaaaagcagaattctcttttatatggtt
tatactgttgatcagaaatgttgattgtgcattgagtattaaaaaattagatgtatattattcattgttctttactcctgagtaccttataataataa
taatgtattctttgttaacaa

All publications, patents, and patent documents are incorporated by reference herein, as though individually incorporated by reference, and in particular, U.S. patenttent Pub. No. U.S. 2021/0395839. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method of detecting Human Papillomavirus (HPV) mediated Squamous Cell Carcinoma (SCC) or cervical cancer in a subject comprising:
extracting polynucleotides from a biological sample from a subject;
amplifying the extracted polynucleotides from the biological sample using a polymerase chain reaction (PCR) method;
detecting target polynucleotides in the amplified polynucleotides from the biological sample, wherein the target polynucleotides comprise one or more of an E6 gene of HPV16, an E7 gene of HPV16, and an E7 gene of HPV18; and
wherein detecting the target polynucleotides indicates the presence of the SCC or the cervical cancer in the subject.

2. The method of claim 1 wherein the target polynucleotides comprise the E6 gene of HPV16, the E7 gene of HPV16, and the E7 gene of the HPV18.

3. The method of claim 1 wherein the biological sample comprises urine, saliva, ascites fluid, vaginal fluid, blood, serum, or plasma.

4. The method of claim 1 wherein the PCR method is reverse transcription quantitative real-time PCR.

5. The method of claim 1 wherein the extracted polynucleotides comprise ribonucleic acid (RNA) or deoxyribonucleic acid (DNA).

6. The method of claim 1 wherein the SCC is Head and Neck Squamous Cell Carcinoma.

7. A method of detecting Human papilloma Virus (HPV) mediated Head and Neck Squamous Cell Carcinoma (HNSCC) or cervical cancer comprising:
extracting deoxyribonucleic acid or ribonucleic acid from a saliva sample from a subject;
subjecting the extracted deoxyribonucleic acid or ribonucleic acid to conditions that amplify the extracted ribonucleic acid using quantitative reverse transcription polymerase chain reaction (RT-qPCR); and
detecting target polynucleotides in the amplified deoxyribonucleic acid or ribonucleic acid, wherein the target polynucleotides comprise an E6 gene of HPV16, an E7 gene of HPV16, and an E7 gene of HPV18;
wherein detection of the target polynucleotides indicates a presence or absence of the HPV mediated HNSCC or the cervical cancer in the biological sample.

8. A method of detecting non-Human Papilloma Virus mediated Squamous Cell Carcinoma (SCC) comprising:
extracting polynucleotides from a biological sample from a subject;
amplifying the polynucleotides in the biological sample using a polymerase chain reaction (PCR) method; and
detecting target polynucleotides in the amplified polynucleotides, wherein the target polynucleotides comprise one or more polynucleotide sequences of TP53, CDKN2A, CCND1, EGFR, PIK3CA, HRAS, KRAS, NRAS, and FBXW7;
wherein a change in expression level of the target polynucleotides or a change in copy number of the target polynucleotides as compared to a biological sample from a reference subject known to be SCC free indicates a presence or absence of the non-HPV mediated SCC in the biological sample.

9. The method of claim 8 wherein the biological sample comprises urine, saliva, ascites fluid, vaginal fluid, blood, serum, or plasma.

10. The method of claim 8 wherein the PCR method is reverse transcription quantitative real-time PCR.

11. The method of claim 8 wherein the change in expression level of the target polynucleotides is determined according to the steps of:
measuring a change in Ct (ΔCt) value for each of the target polynucleotides and a second polynucleotide, wherein the second polynucleotide comprises one or more of RNAseP, 18S, and beta-actin for both the subject and the reference subject known to be SCC free;
calculating the difference in ΔCt value (ΔΔCt) of the subject and the reference subject known to be SCC free obtained from the measuring step;
computing a 2^{-ΔΔCt} to produce a final value for each of the one or more target polynucleotides and the second polynucleotide;
comparing the final value for each of the one or more target polynucleotides to the second polynucleotide to form a comparison ratio, wherein the comparison ratio indicates a fold change in expression of each of the one or more target polynucleotides, and wherein a comparison ratio of 1 or greater indicates the presence of SCC and a comparison ratio of less than 1 indicates the absence of SCC in the biological sample.

12. The method of claim 8 further comprising measuring a fold change in gene copy number of the target polynucleotides, wherein an increase in the gene copy number of the target polynucleotides compared to a wild-type gene copy number of the target polynucleotides indicates the presence of SCC.


SEQUENCE LISTING (105 sequences)

SEQ ID NO: 1 - DNA, 7905 bp, Human papillomavirus 16
  source 1..7905: mol_type = genomic DNA
actacaataatccatgtataaaactaagggcgtaaccgaaatcggttgaaccgaaaccggttagtataaaagcagacattttatgcaccaaaagagaactgcaatgtttcaggacccacaggagcgacccggaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtgtgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcaaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacaatatatgccaaacaccacttacaaatattttaaatgtactaaaaactagtaatgcaaaggcagcaatgttagcaaaatttaaagagttatacggggtgagttttacagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactatatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacgctggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaactattaggcagcacttggccaaccactccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatacacaacattactggcgtgctttttgctttgcttttgtgtgcttttgtgtgtctgcctattaatacgtccgctgcttttgtctgtgtctacatacacatcattaatactattggtattactattgtggataacagcagcctctgcgtttaggtgttttattgtatatattgtatttgtttatataccattatttttaatacatacacatgcacgctttttaattacataatgtatatgtacataatgtaattgttacatataattgttgtataccataacttactattttttcttttttatttttatatataatttttttttggtttgtttgtttgttttttaataaactgttctcacttaacaatgcgacacaaacgttctgcaaaacgcacaaaacgtgcatcggctacccaactttataaaacatgcaaacaggcaggtacatgtccacctgacattatacctaaggttgaaggcaaaactattgctgatcaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccatccccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaagttaaagttgtagaccctgcttttgtaaccactcccactaaacttattacatatgataatcctgcatatgaaggtatagatgtggataatacattatattttcctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttgagtactattgatcctgcagaagaaatagaattacaaactataacaccttctacatatactaccacttcacatgcagcctcacctacttctattaataatggcttatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaaccccccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtttatatgtttgtatgtgcttgtatgtgcttgtaaatattaagttgtatgtgtgtttgtatgtatggtataataaacacgtgtgtatgtgtttttaaatgcttgtgtaactattgtgtgatgcaacataaataaacttattgtttcaacacctactaattgtgttgtggttattcattgtatataaactatatttgctacaatctgtttttgttttatatatactatattttgtagcgccagcggccattttgtagcttcaaccgaattcggttgcatgctttttggcacaaaatgtgtttttttaaatagttctatgtcagcaactatagtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgttttgggttacacatttacaagcaacttatataataatactaa

SEQ ID NO: 2 - DNA, 7857 bp, Human papillomavirus 18
  source 1..7857: mol_type = genomic DNA
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcaaagaaaacagtccattaggggagcggctggaggtggatacagagttaagtccacggttacaagaaatatcgttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaccagcagtgtagacggtacacgtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaattatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagtgcgttgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtgcatacaaaaccgaggattggacactgcaagacacatgcgaggaactatggaatacagaacctactcactgctttaaaaaaggtggccaaacagtacaagtatattttgatggcaacaaagacaattgtatgaactatgtagcatgggacagtgtgtattatatgactgatgcaggaacatgggacaaaacggctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtgtcaggtaatgtatttgttggtacccctacatctggaacacatgggtatgaggaaatacctttacaaacatttgcttcttctggtacaggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaaagggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttctaaatattcgcccactatatcttctgcctcttcctatagtaatgtaacggtccctttaacctcctcttgggatgtgcctgtatacacgggtcctgatattacattaccatctactacctctgtatggcccattgtatcacccacagcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatacaaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 3 - DNA, 7904 bp, Human papillomavirus 16
  source 1..7904: mol_type = genomic DNA
actacaataattcatgtataaaattaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgagataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaagtgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttcccagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtacatgataatgattatttaacacaggcagaaacagagacagcacatgcgttgtttactgcacaggaagcaaaacaacatagagatgcagtacaggttctaaaacgaaagtatttgggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtaagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatgtcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagagtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaataaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacaaacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggaaacatatatatatttgtgaagaagcatcagtaactgtggtagagggtcaagttgactattatggtttatattatgttcatgaaggaatacaaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaactattaggcagcacttggccaaccactccgccgcgacccataccaaagccgtcgccttgggcaccaaagaaacacagacgactatccagcgaccaagatcagagccagacaccggaaacccctgccacaccactaagttgctgcacagagactcagtggacagtgctttaatcctcactgcatttaacagctcacacaaaggacggattaactgtaatagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcaacgtgaacaatttttgtctcaagttaaaataccaaaaactattacagtgtctactggatttatgtctatatgacaaatcttgatactgcatccacaacattactggcgtgctttttgctttgcttttgtgtgcttttgtgtgtctgcctattaatacgtccgctgcttttgtctgtgtctacatacacatcattaatagtattggtattactattgtggataacagcagcctctgcgtttaggtgttttattgtatatattgtatttgtttatataccattatttttaatacatactcatgcacgctttttaattacataatgtatatgtacataatgtaattgttacatataattgttgtataccataacttactattttttttttttatttttatatataattttttgtttgtttgtgtgtttgttttttaataaactgttatcacttaacaatgcgacacaaacgttctgcaaaacgcacaaaacgtgcatcggctacccaactttataaaacatgcaaacaggcaggtacatgtccacctgacattatacctaaggttgaaggcaaaactattgctgatcaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccacttttactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaagttaaagttgtagaccctgcttttgtaaccactcccactaaacttattacatatgataatcctgcatatgaaggtatagatgtggataatacattatattttcctaataatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttcagtactattgatcctgcagaagaaatagaattacaaactataacaccttctacatatactaccacttcacatgcagcctcacctacttctattaataatggattatatgatatttatgcagatgactttattacagatacttttacaaccccggtaccatctgtaccctctacatctttatcaggttatattcctgcaaatacaacaattccttttggtggtgcatacaatattcctttagtatcaggtcctgatatacccattaatataactgaccaagctccttcattacttcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattaaaggccaaaccaaaatttacattaggaaaacgaaaagctacacccaccacctcatctacctctacaactgctaaacgcaaaaaacgtaagctgtaagtattgtatgtatgttgactcagtgttgtttgttgtttatatgtctgtatgtgcttgtatgtgcttgtaaatattacgttgtatgtgtgtttgtatgtatggtataataaacatgtgtgtatgtgtttttcactgcttgtgtaactattgtgtcatgcaacataaataaacttattgtttcaacacctactaattgtgttgtggttattcattgtatataaactatatttgctacatcctgtttttgttttatatatactatattttgtagcgccagcggccattttgtagcttcaaccgaattcggttgcatgctttttggcacaaaatgtgtttttttaaatagttctatgtcagcaactatagtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaccctaattgcatatttggcataaggtttaaacttctaaggccaactaaatgtcaccctagttcatacatgaactgtgtaaaggttagtcatacattgttcatttgtaaaactacacatgggtgtgtgcaaaccgttttgggttacacatttacaagcaacttatataataatactaa

SEQ ID NO: 4 - DNA, 477 bp, Human papillomavirus 16
  source 1..477: mol_type = genomic DNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 5 - DNA, 296 bp, Human papillomavirus 16
  source 1..296: mol_type = genomic DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgacttacgcttcggttgtgcgtacaaagcacacacgtagacattcgtactttggaagacctgttaatgggcacactaggaattgtgtgccccatctgttctcagaaaccataa

SEQ ID NO: 6 - DNA, 318 bp, Human papillomavirus 18
  source 1..318: mol_type = genomic DNA
atgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaa

SEQ ID NO: 7 - DNA, 334 bp, Homo sapiens
  source 1..334: mol_type = other DNA
acaaattctcagatcatcagtcctcacctgagggaccttccgcggcatctatgcgggcatggttactgcctctggtgccccccgcagccgcgcgcaggtaccgtgcgacatcgcgatggcccagctcctcagccaggtccacgggcagacggccccaggcatcgcgcacgtccagccgcgccccggcccggtgcagcaccaccagcgtgtccaggaagccctcccgggcagcgtcgtgcacgggtcgggtgagagtggcggggtcggcgcagttgggctccgcgccgtggagcagcagcagctccgccactcgggcgctgcccatcatcatgac

SEQ ID NO: 8 - DNA, 3423 bp, Homo sapiens
  source 1..3423: mol_type = genomic DNA
gagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccaccccgaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccactcaggaggctgaggtgggaagatcacttgaggccaggagatggaggctgcagtgagctgtgatcacaccactgtgctccagcctgagtgacagagcaagaccctatctcaaaaaaaaaaaaaaagaaaagctcctgaggtgtagacgccaactctctctagctcgctagtgggttgcaggaggtgcttacacatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccgggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagagaaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaa

SEQ ID NO: 9 - DNA, 9256 bp, Homo sapiens
  source 1..9256: mol_type = genomic DNA
agttccggtgccgccgctgcggccgctgaggtgtcgggctgctgctgccgcggccgctgggactggggctggggccgccggcgaggcagggctcgggcccggccgggcagctccggagcggcgggggagaggggccgggaggcgggggccgtgccgcccgctctcctctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagataggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaaaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttattttcaaaagttactacaaccaaattaattctattagaagaaatgtagacaaattctataaagactatagattgtgacctaagaaagaaatgaggcaaagaaccaaacattgaattaaatgctacatgggtgactaagatctgtttcaagtcagtgataatatagccacttctgggtacttcagtatcagagatcagttctcgtggtttagacagttcctatctatagctgactatccttgtccttgaatatggtgtaactgactattggctctacagttttattgggccacttaagaaatatttccttgaataattattttgagaaaaagtctaaaagtaataaaaataattttaaacacactgtagtaagaaatgactgttggaaaattatgctttcactttctaccatattctcagctatacaaaaccatttattttgaagatttttagactactgttaatttgaaatctgttactcttattgtggaatttgtttttttaaaaaagatgtttctaattggatttttaaaagaagaatggaatttggttgctattttacaatagaacctaagctttttgtggttcttagtgtcctatgtaaaacttagtgtcaaagtaatcaactttgagattttcccttctattctgctttatattaaaagcccattagaaaatgggaacctggtgaatatataatgaattgtaaaatattttaatgtgtaactttttcaactgtgaaactgacttgattttttgatgaaaacagctgctgataaagtattttgtgtaaagtgtagttcttattaatcaggaaaatgatgacttgattagactgtatatgccctcttggattttattttaaatggattggtgactttcacataggtaaaacacagtccatctgtattcttttttccatcaaaaatcgagtgatttggaattataaaaaaattgtgagcagcctatttgaaaggcatcatggaaatttcacagcacaataacacggatttgttttttcttaatgatgtaaatccgtttaattcatactttgatcaatagcccatgcttgccaactctgaagaaatttaatttccagcagtattttaaagctagcctgttaactttttctgaatatttaaagttcctcttttttctatgtctgcacaaactgcagacctgggctggacccacatactcaagagtccaccttaagaaattattttgatgtccaagacatcactaaaatatttaagtttaaagataatatgtggtgttaatagattgtggtgcttttactatttaaagacaactttcatacttcagatgtttttgagaagaggggaatgtgaggggagggggcagaacagggaggagttgtttgaatgaattacattctttatatccatcctgctcatttggggcatgtctttaagagaaggctgaaagttgtgagagtatattgtataccgtaagagaatcaactcttcatcatggatgggattgtgaaggctgaactataaaattcagcattgacagcatcctcaattaataattcttggtgacagaataatacagctgggctgttttttaaaatataaacaataccatttttaattattacattaaaaattgtaaatatatctatgtgccatggcctgggagcctgctttcttttttcataaaaattatttttactgtatgaaaagatcatggggtttagctcaaaatatctgtggtcctgataaaattggattggtaactctacctcagaaggaaaatgggaaaaaaaaatagatgagtcacaattcaatacttcaagctcagaaactgtgcagatcactgaattttagatttataaagtcagagttggcatgccttgtttttaatgatatggaagaccttaagaaaaaaacttggctgaagtttaatcgttggtccagccatttgaaaaaggcaatagtttgaggaggttcccgaattcggcatttgaaattcattttgttctctcttcttcattattagtgcatttggtgtgtgtatacttgcacacaattctgtttgtgtacacactgcttgcttagccctagtcaagaggcatcttttataaaaggtgtaaagaaatatcaaggttctaaaattcggaagagtttagaatttattaggagtttcccaagttgggatgttagtctttaaataaacttcatgcacctattccacttaaggttttgcacctcctttttattagtgcagtgccatttcttctgcttgattttaggtatgttaatattccagccttgctagttagcataaagtgacaggtgtgagccatgaggaaattttctgacttaatttgtacacaactacatataagagttttagtggaggaaaaaaattagtcccttgtgcgtatacagtagttaggtaaatgatttttctaccaacagtatactccattcctcatgtaggtaagtacagaaaaggtttttaaatgtatttttttagccagttaaagtctatgaatctatctgcaaccttatttaatctgtcactataataattttgtggttatgctaagaaccatgtatacttttaggtattcttatttttgtcaatttttctaggttggcaaggaggcagaaaaccttcattgtttcatattaaaatataattagactaaacttaattctagtatgaatttccaaaatcattatctatttatttcatttttatttaattttgtttttatttcatttttaaaagtcccttgttcaatttaacttatgttcctaagagaggttggagaacttggccttcatctgatttcaaaaatgttttgagtttcaaatgaagttaatggtttcagtgtgattcagtcctcagacctaattgggttgaataaaatctaaaagaatatacccttttggagcataacattttaataccttggggaatgtggcactaccaaaagaagactactaacacgtcagatgttcacctggaagctttatcaagaaattcgaaccacccttttggccccattaattgtagcaagtttatttctctatattttgtcattcagtgaattgaagtcctgtggtatactgcattcattagaagaaaaacgtttttaatgtccttttaatgatggcccagaaagcatttgacacagcaagatgcatgtgttactatattgagaatatagaataataacagtatcactaaatttaagacctcttcccagtcttgctgttcctagcaagaagtttggcctgtgactgcacttactgtttatgctcatcagaaactgtcaatgtctgcttttctttaactctgcagtctgtaacatcacgctgtttattaaaaaaaaaaagaaaaatta

SEQ ID NO: 10 - DNA, 953 bp, Homo sapiens
  source 1..953: mol_type = other DNA
gcagtcgcgcctgtgaacggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggcagccgctctggctctagctccagctccgggaccctctgggaccccccgggacccatgtgacccagcggcccctcgcgctggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggctctgtgggcccttgtcgggcacagatgggatcacagtaaattattggatggtcttga

SEQ ID NO: 11 - DNA, 1070 bp, Homo sapiens
  source 1..1070: mol_type = other DNA
aggcccgcccgagtctccgccgcccgtgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttga

SEQ ID NO: 12 - DNA, 567 bp, Homo sapiens
  source 1..567: mol_type = other DNA
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagacggtaaaaagaagaaaaagaagtcaaagacaaagtgtgtaattatgtaa

SEQ ID NO: 13 - DNA, 567 bp, Homo sapiens; identical to SEQ ID NO: 12
  source 1..567: mol_type = other DNA

SEQ ID NO: 14 - DNA, 3926 bp, Homo sapiens
  source 1..3926: mol_type = other DNA
taccgcgccggagccttccgcagctgccgcttcagtccgaaggaggaagggaaccaacccactttctcggcgccgcggctcttttctaaaagtaatgtgaaaacctttgcatcttctgatagtctagccaaggtccaagaagtagcaagctggcttttggaaatgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattgggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttttttttttgccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattatttttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgtttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgttaaaaaaaaaaaaaaaaaaa

SEQ ID NO: 15 - DNA, 4243 bp, Homo sapiens
  source 1..4243: mol_type = other DNA
agcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccagactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctccactgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtggccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccaaaaaaaaaaaaaaaaaaaaaaaaaaaa

SEQ ID NO: 16 - DNA, 9905 bp, Homo sapiens
  source 1..9905: mol_type = other DNA
agacgtccgggcagcccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctattatgctctcaaatacccctaagcatctatactagcctggtatgggtatgaaagatacaaagataaataaaacatagtccctgattctaagaaattcacaatttagcaaaggaaatggactcatagatgctaaccttaaaacaacgtgacaaatgccagacaggacccatcagccaggcactgtgagagcacagagcagggaggttgggtcctgcctgaggagacctggaagggaggcctcacaggaggatgaccaggtctcagtcagcggggaggtggaaagtgcaggtgcatcaggggcaccctgaccgaggaaacagctgccagaggcctccactgctaaagtccacataaggctgaggtcagtcaccctaaacaacctgctccctctaagccaggggatgagcttggagcatcccacaagttccctaaaagttgcagcccccagggggattttgagctatcatctctgcacatgcttagtgagaagactacacaacatttctaagaatctgagattttatattgtcagttaaccactttcattattcattcacctcaggacatgcagaaatatttcagtcagaactgggaaacagaaggacctacattctgctgtcacttatgtgtcaagaagcagatgatcgatgaggcaggtcagttgtaagtgagtcacattgtagcattaaattctagtatttttgtagtttgaaacagtaacttaataaaagagcaaaagctattctagctttcttcttcatattttaattttccaccataaagtttagttgctaaattctattaattttaagattgtgcttcccaaaatagttctcacttcatctgtccagggaggcacagttctgtctggtagaagccgcaaagcccttagcctcttcacggatctggcgactgtgatgggcaggtcaggagaggagctgcccaaagtcccatgattttcacctaacagccctgatcagtcagtactcaaagcttggactccatccctgaaggtcttcctgattgatagcctggccttaataccctacagaaagcctgtccattggctgtttcttcctcagtcagttcctggaagaccttaccccatgaccccagcttcagatgtggtctttggaaacagaggtcgaaggaaagtaaggagctgagagctcacattcataggtgccgccagccttcgtgcatcttcttgcatcatctctaaggagctcctctaattacaccatgcccgtcaccccatgagggatcagagaagggatgagtcttctaaactctatattcgctgtgagtccaggttgtaagggggagcactgtggatgcatcctattgcactccagctgatgacaccaaagcttaggtgtttgctgaaagttcttgatgttgtgacttaccacccctgcctcacaactgcagacataaggggactatggattgcttagcaggaaaggcactggttctcaagggcggctgcccttgggaatcttctggtcccaaccagaaagactgtggcttgattttctcaggtgcagcccagccgtagggccttttcagagcaccccctggttattgcaacattcatcaaagtttctagaacctctggcctaaaggaagggcctggtgggatctacttggcactcgctggggggccaccccccagtgccactctcactaggcctctgattgcacttgtgtaggatgaagctggtgggtgatgggaactcagcacctcccctcaggcagaaaagaatcatctgtggagcttcaaaagaaggggcctggagtctctgcagaccaattcaacccaaatctcgggggctctttcatgattctaatgggcaaccagggttgaaacccttatttctagggtcttcagttgtacaagactgtgggtctgtaccagagcccccgtcagagtagaataaaaggctgggtagggtagagattcccatgtgcagtggagagaacaatctgcagtcactgataagcctgagacttggctcatttcaaaagcgttcaattcatcctcaccagcagttcagctggaaaggggcaaatacccccacctgagctttgaaaacgccctgggaccctctgcattctctaagtaagttatagaaaccagtctcttccctcctttgtgagtgagctgctattccacgtaggcaacacctgttgaaattgccctcaatgtctactctgcatttctttcttgtgataagcacacacttttattgcaacataatgatctgctcacatttccttgcctgggggctgtaaaaccttacagaacagaaatccttgcctctttcaccagccacacctgccataccaggggtacagctttgtactattgaagacacagacaggatttttaaatgtaaatctatttttgtaactttgttgcgggatatagttctctttatgtagcactgaactttgtacaatatatttttagaaactcatttttctactaaaacaaacacagtttactttagagagactgcaatagaatcaaaatttgaaactgaaatctttgtttaaaagggttaagttgaggcaagaggaaagccctttctctctcttataaaaaggcacaacctcattggggagctaagctaggtcattgtcatggtgaagaagagaagcatcgtttttatatttaggaaattttaaaagatgatggaaagcacatttagcttggtctgaggcaggttctgttggggcagtgttaatggaaagggctcactgttgttactactagaaaaatccagttgcatgccatactctcatcatctgccagtgtaaccctgtacatgtaagaaaagcaataacatagcactttgttggtttatatatataatgtgacttcaatgcaaattttatttttatatttacaattgatatgcatttaccagtataaactagacatgtctggagagcctaataatgttcagcacactttggttagttcaccaacagtcttaccaagcctgggcccagccaccctagagaagttattcagccctggctgcagtgacatcacctgaggagcttttaaaagcttgaagcccagctacacctcagaccgattaaacgcaaatctctggggctgaaacccaagcattcgtagtttttaaagctcctgaggtcattccaatgtgcggccaaagttgagaactactggcctagggattagccacaaggacatggacttggaggcaaattctgcaggtgtatgtgattctcaggcctagagagctaagacacaaagacctccacatctgtcgctgagagtcaagaacctgaacagagtttccatgaaggttctccaagcactagaagggagagtgtctaaacaatggttgaaaagcaaaggaaatataaaacagacacctctttccatttcctaaggtttctctctttattaagggtggactagtaataaaatataatattcttgctgcttatgcagctgacattgttgccctccctaaagcaaccaagtagcctttatttcccacagtgaaagaaaacgctggcctatcagttacattacaaaaggcagatttcaagaggattgagtaagtagttggatggctttcataaaaacaagaattcaagaagaggattcatgctttaagaaacatttgttatacattcctcacaaattatacctgggataaaaactatgtagcaggcagtgtgttttccttccatgtctctctgcactacctgcagtgtgtcctctgaggctgcaagtctgtcctatctgaattcccagcagaagcactaagaagctccaccctatcacctagcagataaaactatggggaaaacttaaatctgtgcatacatttctggatgcatttacttatctttaaaaaaaaaggaatcctatgacctgatttggccacaaaaataatcttgctgtacaatacaatctcttggaaattaagagatcctatggatttgatgactggtattagaggtgacaatgtaaccgattaacaacagacagcaataacttcgttttagaaacattcaagcaatagctttatagcttcaacatatggtacgttttaaccttgaaagttttgcaatgatgaaagcagtatttgtacaaatgaaaagcagaattctcttttatatggtttatactgttgatcagaaatgttgattgtgcattgagtattaaaaaattagatgtatattattcattgttctttactcctgagtaccttataataataataatgtattctttgttaacaa

SEQ ID NO: 17 - DNA, 25 bp, Synthetic construct
  source 1..25: mol_type = other DNA
acaaattctcagatcatcagtcctc

SEQ ID NO: 18 - DNA, 16 bp, Synthetic construct
  source 1..16: mol_type = other DNA
aggagctgggccatcg

SEQ ID NO: 19 - DNA, 17 bp, Synthetic construct
  source 1..17: mol_type = other DNA
gggtcgggtgagagtgg

SEQ ID NO: 20 - DNA, 21 bp, Synthetic construct
  source 1..21: mol_type = other DNA
tggctctgaccattctgttct

SEQ ID NO: 21 - DNA, 21 bp, Synthetic construct
  source 1..21: mol_type = other DNA
tcatcttgggcctgtgttatc

SEQ ID NO: 22 - DNA, 20 bp, Synthetic construct
  source 1..20: mol_type = other DNA
tgtgatgatggtgaggatgg

SEQ ID NO: 23 - DNA, 22 bp, Synthetic construct
  source 1..22: mol_type = other DNA
tggctctgactgtaccaccatc

SEQ ID NO: 24 - DNA, 18 bp, Synthetic construct
  source 1..18: mol_type = other DNA
gtggcaagtggctcctga

SEQ ID NO: 25 - DNA, 20 bp, Synthetic construct
  source 1..20: mol_type = other DNA
ccccctccatcaacttcttc

SEQ ID NO: 26 - DNA, 20 bp, Synthetic construct
  source 1..20: mol_type = other DNA
gaaaaagccgaaggtcacaa

SEQ ID NO: 27 - DNA, 23 bp, Synthetic construct
  source 1..23: mol_type = other DNA
tttgatgacattgcatacattcg

SEQ ID NO: 28 - DNA, 25 bp, Synthetic construct
  source 1..25: mol_type = other DNA
gatccaatccatttttgttgtccag

SEQ ID NO: 29 - DNA, 20 bp, Synthetic construct
  source 1..20: mol_type = other DNA
gtggtcattgatggggagac

SEQ ID NO: 30 - DNA, 20 bp, Synthetic construct
  source 1..20: mol_type = other DNA
gatggcaaacacacacagga

SEQ ID NO: 31 - DNA, 20 bp, Synthetic construct
  source 1..20: mol_type = other DNA
gacggaatataagctggtgg

SEQ ID NO: 32 - DNA, 20 bp, Synthetic construct
  source 1..20: mol_type = other DNA
aggcacgtctccccatcaat

SEQ ID NO: 33 - DNA, 21 bp, Synthetic construct
  source 1..21: mol_type = other DNA
acacccccaggattcttacag

SEQ ID NO: 34 - DNA, 20 bp, Synthetic construct
  source 1..20: mol_type = other DNA
cgcctgtcctcatgtattgg

SEQ ID NO: 35 - DNA, 20 bp, Synthetic construct
  source 1..20: mol_type = other DNA
gatgtggctcgccaattaac

SEQ ID NO: 36 - DNA, 20 bp, Synthetic construct
  source 1..20: mol_type = other DNA
gattgtcagtgcgcttttcc

SEQ ID NO: 37 - DNA, 20 bp, Synthetic construct
  source 1..20: mol_type = other DNA
tcaaagaatggtcctggacc

SEQ ID NO: 38 - DNA, 25 bp, Synthetic construct
  source 1..25: mol_type = other DNA
atgactgaatataaacttgtggtag

SEQ ID NO: 39 - DNA, 28 bp, Synthetic construct
  source 1..28: mol_type = other DNA
ttacataattacacactttgtctttgac

SEQ ID NO: 40 - DNA, 23 bp, Synthetic construct
  source 1..23: mol_type = other DNA
ttgagacaggccagtgtttacat

SEQ ID NO: 41 - DNA, 20 bp, Synthetic construct
  source 1..20: mol_type = other DNA
cagtctctggatcccacacc

SEQ ID NO: 42 - DNA, 20 bp, Synthetic construct
  source 1..20: mol_type = other DNA
gcagaaggaggtcctgccgt

SEQ ID NO: 43 - DNA, 20 bp, Synthetic construct
  source 1..20: mol_type = other DNA
gtcacacttgatcactctgg

SEQ ID NO: 44 - DNA, 18 bp, Synthetic construct
  source 1..18: mol_type = other DNA
ccgtccatgcggaagatc

SEQ ID NO: 45 - DNA, 20 bp, Synthetic construct
  source 1..20: mol_type = other DNA
gaagacctcctcctcgcact

SEQ ID NO: 46 - DNA, 21 bp, Synthetic construct
  source 1..21: mol_type = other DNA
atgcaaataaaaccggactga

SEQ ID NO: 47 - DNA, 20 bp, Synthetic construct
  source 1..20: mol_type = other DNA
ccagcctctcaccctgtaaa

SEQ ID NO: 48 - DNA, 22 bp, Synthetic construct
  source 1..22: mol_type = other DNA
tgactgaggacagcatagacga

SEQ ID NO: 49 - DNA, 22 bp, Synthetic construct
  source 1..22: mol_type = other DNA
gggctggacagtgttgagatac

SEQ ID NO: 50 - DNA, 20 bp, Synthetic construct
  source 1..20: mol_type = other DNA
catgtcgatggacttccaga

SEQ ID NO: 51 - DNA, 20 bp, Synthetic construct
  source 1..20: mol_type = other DNA
gggacagcttggatcacact

SEQ ID NO: 52 - DNA, 19 bp, Synthetic construct
  source 1..19: mol_type = other DNA
agatttggacctgcgagcg

SEQ ID NO: 53 - DNA, 20 bp, Synthetic construct
  source 1..20: mol_type = other DNA
gagcggctgtctccacaagt

SEQ ID NO: 54 - DNA, 20 bp, Synthetic construct
  source 1..20: mol_type = other DNA
gatgaaatagatggtccagc

SEQ ID NO: 55 - DNA, 22 bp, Synthetic construct
  source 1..22: mol_type = other DNA
gctttgtacgcacaaccgaagc

SEQ ID NO: 56 - DNA, 20 bp, Synthetic construct
  source 1..20: mol_type = other DNA
gcaatgtttcaggacccaca

SEQ ID NO: 57 - DNA, 22 bp, Synthetic construct
  source 1..22: mol_type = other DNA
cgcagtaactgttgcttgcagt

SEQ ID NO: 58 - DNA, 21 bp, Synthetic construct
  source 1..21: mol_type = other DNA
tgtgactctacgcttcggttg

SEQ ID NO: 59 - DNA, 20 bp, Synthetic construct
  source 1..20: mol_type = other DNA
gcccattaacaggtcttcca

SEQ ID NO: 60 - DNA, 20 bp, Synthetic construct
  source 1..20: mol_type = other DNA
gcatggacctaaggcaacat

SEQ ID NO: 61 - DNA, 21 bp, Synthetic construct
  source 1..21: mol_type = other DNA
gaaggtcaaccggaatttcat

SEQ ID NO: 62 - DNA, 20 bp, Synthetic construct
  source 1..20: mol_type = other DNA
gtaacccgttgaaccccatt

SEQ ID NO: 63 - DNA, 20 bp, Synthetic construct
  source 1..20: mol_type = other DNA
ccatccaatcggtagtagcg

SEQ ID NO: 64 - DNA, 25 bp, Synthetic construct
  source 1..25: mol_type = other DNA
tgacattaaggagaagctgtcctac

SEQ ID NO: 65 - DNA, 24 bp, Synthetic construct
  source 1..24: mol_type = other DNA
gagttgaaggtagtttcgtggatg

SEQ ID NO: 66 - DNA, 20 bp, Synthetic construct
  source 1..20: mol_type = other DNA
gatgaaatagatggtccagc

SEQ ID NO: 67 - DNA, 22 bp, Synthetic construct
  source 1..22: mol_type = other DNA
gctttgtacgcacaaccgaagc

SEQ ID NO: 68 - DNA, 20 bp, Synthetic construct
  source 1..20: mol_type = other DNA
gcaatgtttcaggacccaca

SEQ ID NO: 69 - DNA, 22 bp, Synthetic construct
  source 1..22: mol_type = other DNA
cgcagtaactgttgcttgcagt

SEQ ID NO: 70 - DNA, 21 bp, Synthetic construct
  source 1..21: mol_type = other DNA
tgtgactctacgcttcggttg

SEQ ID NO: 71 - DNA, 20 bp, Synthetic construct
  source 1..20: mol_type = other DNA
gcccattaacaggtcttcca

SEQ ID NO: 72 - DNA, 20 bp, Synthetic construct
  source 1..20: mol_type = other DNA
gcatggacctaaggcaacat

SEQ ID NO: 73 - DNA, 21 bp, Synthetic construct
  source 1..21: mol_type = other DNA
gaaggtcaaccggaatttcat

SEQ ID NO: 74 - DNA, 25 bp, Synthetic construct
  source 1..25: mol_type = other DNA
acaaattctcagatcatcagtcctc

SEQ ID NO: 75 - DNA, 16 bp, Synthetic construct
  source 1..16: mol_type = other DNA
aggagctgggccatcg

SEQ ID NO: 76 - DNA, 21 bp, Synthetic construct
  source 1..21: mol_type = other DNA
tcatcttgggcctgtgttatc

SEQ ID NO: 77 - DNA, 20 bp, Synthetic construct
  source 1..20: mol_type = other DNA
tgtgatgatggtgaggatgg

SEQ ID NO: 78 - DNA, 22 bp, Synthetic construct
  source 1..22: mol_type = other DNA
tggctctgactgtaccaccatc

SEQ ID NO: 79 - DNA, 18 bp, Synthetic construct
  source 1..18: mol_type = other DNA
gtggcaagtggctcctga

SEQ ID NO: 80 - DNA, 20 bp, Synthetic construct
  source 1..20: mol_type = other DNA
ccccctccatcaacttcttc

SEQ ID NO: 81 - DNA, 20 bp, Synthetic construct
  source 1..20: mol_type = other DNA
gaaaaagccgaaggtcacaa

SEQ ID NO: 82 - DNA, 23 bp, Synthetic construct
  source 1..23: mol_type = other DNA
tttgatgacattgcatacattcg

SEQ ID NO: 83 - DNA, 25 bp, Synthetic construct
  source 1..25: mol_type = other DNA
gatccaatccatttttgttgtccag

SEQ ID NO: 84 - DNA, 20 bp, Synthetic construct
  source 1..20: mol_type = other DNA
gtggtcattgatggggagac

SEQ ID NO: 85 - DNA, 20 bp, Synthetic construct
  source 1..20: mol_type = other DNA
gatggcaaacacacacagga

SEQ ID NO: 86 - DNA, 20 bp, Synthetic construct
  source 1..20: mol_type = other DNA
gacggaatataagctggtgg

SEQ ID NO: 87 - DNA, 20 bp, Synthetic construct
  source 1..20: mol_type = other DNA
aggcacgtctccccatcaat

SEQ ID NO: 88 - DNA, 21 bp, Synthetic construct
  source 1..21: mol_type = other DNA
acacccccaggattcttacag

SEQ ID NO: 89 - DNA, 20 bp, Synthetic construct
  source 1..20: mol_type = other DNA
cgcctgtcctcatgtattgg

SEQ ID NO: 90 - DNA, 20 bp, Synthetic construct
  source 1..20: mol_type = other DNA
gatgtggctcgccaattaac

SEQ ID NO: 91 - DNA, 20 bp, Synthetic construct
  source 1..20: mol_type = other DNA
gattgtcagtgcgcttttcc

SEQ ID NO: 92 - DNA, 25 bp, Synthetic construct
  source 1..25: mol_type = other DNA
atgactgaatataaacttgtggtag

SEQ ID NO: 93 - DNA, 28 bp, Synthetic construct
  source 1..28: mol_type = other DNA
ttacataattacacactttgtctttgac

SEQ ID NO: 94 - DNA, 23 bp, Synthetic construct
  source 1..23: mol_type = other DNA
ttgagacaggccagtgtttacat

SEQ ID NO: 95 - DNA, 20 bp, Synthetic construct
  source 1..20: mol_type = other DNA
cagtctctggatcccacacc

SEQ ID NO: 96 - DNA, 20 bp, Synthetic construct
  source 1..20: mol_type = other DNA
gcagaaggaggtcctgccgt

SEQ ID NO: 97 - DNA, 20 bp, Synthetic construct
  source 1..20: mol_type = other DNA
gtcacacttgatcactctgg

SEQ ID NO: 98 - DNA, 18 bp, Synthetic construct
  source 1..18: mol_type = other DNA
ccgtccatgcggaagatc

SEQ ID NO: 99 - DNA, 20 bp, Synthetic construct
  source 1..20: mol_type = other DNA
gaagacctcctcctcgcact

SEQ ID NO: 100 - DNA, 21 bp, Synthetic construct
  source 1..21: mol_type = other DNA
atgcaaataaaaccggactga

SEQ ID NO: 101 - DNA, 20 bp, Synthetic construct
  source 1..20: mol_type = other DNA
gggacagcttggatcacact

SEQ ID NO: 102 - DNA, 22 bp, Synthetic construct
  source 1..22: mol_type = other DNA
tgactgaggacagcatagacga

SEQ ID NO: 103 - DNA, 22 bp, Synthetic construct
  source 1..22: mol_type = other DNA
gggctggacagtgttgagatac

SEQ ID NO: 104 - DNA, 20 bp, Synthetic construct
  source 1..20: mol_type = other DNA
catgtcgatggacttccaga

SEQ ID NO: 105 - DNA, 20 bp, Synthetic construct
  source 1..20: mol_type = other DNA
gggacagcttggatcacact